SEQUENCE LISTING (12 sequences)

SEQ ID NO: 1 - protein, 122 aa, synthetic construct
QVQLQQSGAEVKKPGASVKVSCKASGYTFTSYGISWVRQAPGQGLEWMGWISAYNGNTNYAQKLQGRVTMTTDTSTSTAYMELRSLRSDDTAVYYCARNGLWGWDSDAFDIWGRGTLVTVSS

SEQ ID NO: 2 - protein, 110 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTVVFGGGTKVTVL

SEQ ID NO: 3 - protein, 5 aa, synthetic construct
SYGIS

SEQ ID NO: 4 - protein, 17 aa, synthetic construct
WISAYNGNTNYAQKLQG

SEQ ID NO: 5 - protein, 13 aa, synthetic construct
NGLWGWDSDAFDI

SEQ ID NO: 6 - protein, 14 aa, synthetic construct
TGTSSDVGGYNYVS

SEQ ID NO: 7 - protein, 7 aa, synthetic construct
DVSNRPS

SEQ ID NO: 8 - protein, 10 aa, synthetic construct
SSYTSSSTVV

SEQ ID NO: 9 - protein, 216 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTVVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - protein, 452 aa, synthetic construct
QVQLQQSGAEVKKPGASVKVSCKASGYTFTSYGISWVRQAPGQGLEWMGWISAYNGNTNYAQKLQGRVTMTTDTSTSTAYMELRSLRSDDTAVYYCARNGLWGWDSDAFDIWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 366 bp, synthetic construct
  source 1..366: mol_type = other DNA
caggtacagctgcagcagtcaggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagaaatggcctctgggggtgggactctgatgcttttgatatctggggccgaggaaccctggtcaccgtctcctca

SEQ ID NO: 12 - DNA, 330 bp, synthetic construct
  source 1..330: mol_type = other DNA
cagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcactgtggtattcggcggagggaccaaggtcaccgtccta

(19) United States
(12) Patent Application Publication
(10) Pub. No.: US 2026/0027212 A1
(43) Pub. Date: Jan. 29, 2026

(54) FORMULATIONS COMPRISING AN IMMUNOGLOBULIN-LIKE TRANSCRIPT 7 (ILT7)-BINDING PROTEIN

(71) Applicant: Viela Bio, Inc., Deerfield, IL (US)

(72) Inventors: Roberto DEPAZ, Bethesda, MD (US); Jennifer CASIANO-MATOS, Clarksburg, MD (US)

(73) Assignee: Viela Bio, Inc., Deerfield, IL (US)

(21) Appl. No.: 18/997,755

(22) PCT Filed: Jul. 27, 2023
(86) PCT No.: PCT/US2023/071090

§ 371 Date: Jan. 22, 2025

Related U.S. Application Data
(60) Provisional application No. 63/369,567, filed on Jul. 27, 2022.

(51) Int. Cl. A61K 47/18; A61K 47/02; A61K 47/12; A61K 47/26; C07K 16/28

(52) U.S. Cl. CPC A61K 47/183; A61K 47/02; A61K 47/12; A61K 47/26; C07K 16/2803; C07K 2317/52; C07K 2317/565

(57) ABSTRACT

Provided are immunoglobulin-like transcript 7 (ILT7)-binding proteins and methods of making, utilizing, and storing the same. Also provided are improved formulations comprising anti-ILT7 binding proteins and devices comprising the disclosed formulations.

### CROSS-REFERENCE TO RELATED APPLICATIONS

This application claims priority to U.S. Application No. 63/369,567 filed on Jul. 27, 2022, which is hereby incorporated by reference in its entirety for all purposes.

### REFERENCE TO AN ELECTRONIC SEQUENCE LISTING

The contents of the electronic sequence listing (HOPA_037_01WO_SeqList_ST26.xml; Size: 13,040 bytes; and Date of Creation: Jul. 14, 2023) are herein incorporated by reference in its entirety.

### FIELD OF THE DISCLOSURE

The provided disclosure relates to improved immunoglobulin-like transcript 7 (ILT7)-binding proteins and methods of making, utilizing, and storing the same. Also provided are devices comprising the described proteins and methods of use thereof to treat various disease and conditions associated with ILT7-expressing cells.

### BACKGROUND

Proteins can undergo a variety of degradation processes, including aggregation, deamidation, isomerization, oxidation, disulfide bond scrambling, and truncation. These events can occur during fermentation, purification, formulation, manufacturing, and storage. The protein's clinical utility and shelf-life is compromised if the degradation adversely affects the biological activity of the protein or induces immunogenicity. One of the most common degradation processes observed is oxidation of labile amino acid residues.

Light-induced tryptophan oxidation in complementarity-determining regions (CDRs) negatively impacts potency and shelf life of immunoglobulin-like transcript 7 (ILT7) binding proteins. Therefore, there is a need to address the oxidation of affected residues in ILT7 binding proteins to maintain their integrity for use. Moreover, there is also a need to eliminate or reduce the need for engineering controls that include but are not limited to lighting and the need for a nitrogen overlay during manufacturing of these proteins.

The present disclosure addresses these and more limitations of the state-of-the art and provides various improved compositions and methods.

### BRIEF SUMMARY

The compositions and methods of the disclosure provide improvements over the state-of-the-art and resolve limitations associated with existing manufacturing methods. Specifically, existing methods are limited by the emergence of light-induced tryptophan oxidation in complementarity-determining regions (CDRs) of antibodies that negatively impacts potency and shelf life.

Moreover, provided herein are solutions that allow for delivery of higher antibody concentrations which were previously limited due to increased aggregation rate and formulation viscosity. Indeed, the methods of the disclosure provide for improved formulations that contain higher concentrations of ILT7 binding proteins that allow for more convenient administration regimens at higher doses.

Further, provided methods obviate the need for engineering controls such as specific lighting conditions and inclusion of a nitrogen overlay during manufacture thereby reducing manufacturing costs, work costs, and overhead associated with antibody manufacture.

Provided herein are compositions, comprising: (i) about 100 mg/mL to about 350 mg/mL of an anti-immunoglobulin-like transcript 7 (ILT7) binding protein; (ii) about 50 to about 350 mM sucrose; (iii) about 0.001% to about 1% polysorbate-80; (iv) L-methionine; and (v) a buffer selected from a histidine buffer, a citrate buffer, or a phosphate buffer.

Provided herein are compositions, comprising: (i) about 100 mg/mL to about 165 mg/mL of an anti-immunoglobulin-like transcript 7 (ILT7) binding protein; (ii) about 150 to about 240 mM sucrose; (iii) about 0.003% to about 0.8% polysorbate-80; (iv) L-methionine; and (v) a buffer selected from a histidine buffer, a citrate buffer, or a phosphate buffer.

In embodiments, a buffer in a composition is a histidine buffer. In embodiments, a buffer is a phosphate buffer. In embodiments, a buffer is a citrate buffer. In embodiments, a pH of a composition is acidic as determined by a pH meter. In embodiments, an acidic pH is from about 5 to about 7 as determined by pH meter. In embodiments, an acidic pH of a composition of the disclosure is about 6 as determined by pH meter. In embodiments, a composition comprises from about 5 mM to about 25 mM histidine/histidine-HCl. In embodiments, a composition comprises 20 mM histidine/histidine-HCl. In embodiments, a composition comprises about 5 mM to about 50 mM L-methionine. In embodiments, a composition comprises 15 mM L-methionine. In embodiments, a composition comprises 0.02% polysorbate-80. In embodiments, a composition comprises up to about 150 mg/mL of an ILT7 binding protein. In embodiments, a composition comprises about 150 mg/mL of an anti-ILT7 binding protein. In embodiments, an anti-ILT7 binding protein comprises: i. a VH comprising at least 95%, 96%, 97%, 98% or 99% identity to SEQ ID NO: 1; ii. a VL comprising at least 95%, 96%, 97%, 98% or 99% identity to SEQ ID NO: 2; or iii. both i. and ii. In embodiments, an anti-ILT7 binding protein comprises: i. a VH comprising SEQ ID NO: 1; ii. a VL comprising SEQ ID NO: 2; or iii. both i. and ii. In embodiments, an anti-ILT7 binding protein comprises Complementarity-Determining Regions (CDRs) HCDR1, HDR2, HCDR3, LCDR1, LCDR2, and LCDR3 comprising the sequences of SEQ ID NOs: 3, 4, 5, 6, 7, and 8, respectively. In embodiments, a composition comprises a viscosity of about 5 cP to about 50 cP as determined by a viscometer. In embodiments, an anti-ILT7 binding protein is daxdilimab. Daxdilimab can also be referred to as HZN7734, VIB7734, or MEDI7734.

Provided are compositions, comprising about: i. 150 mg/mL of an anti-immunoglobulin-like transcript 7 (ILT7) binding protein; ii. 20 mM histidine/histidine-HCl; iii. 180 mM Sucrose; iv. 15 mM L-Methionine; and v. 0.02% Polysorbate-80.

Provided are compositions, comprising about: i. 150 mg/ml of an anti-immunoglobulin-like transcript 7 (ILT7) binding protein; ii. 20 mM histidine/histidine-HCl; iii. 180 mM Sucrose; iv. 15 mM L-Methionine; and v. 0.02% Polysorbate-80, wherein the ILT7 binding protein comprises: a VH comprising SEQ ID NO: I and a VL comprising SEQ ID NO: 2.

Provided are compositions, comprising about: i. 150 mg/ml of an anti-immunoglobulin-like transcript 7 (ILT7) binding protein; ii. 20 mM histidine/histidine-HCl; iii. 180 mM Sucrose; iv. 15 mM L-Methionine; and v. 0.02% Polysorbate-80, wherein the ILT7 binding protein comprises Complementarity-Determining Regions (CDRs) HCDR1, HDR2, HCDR3, LCDR1, LCDR2, and LCDR3 comprising the sequences of SEQ ID NOs: 3, 4, 5, 6, 7, and 8, respectively.

In embodiments, viscosity of a composition is from about 5 to about 50 cP. In embodiments, viscosity of a composition does not exceed 20 cP. In embodiments, rate of aggregation of a composition is reduced as compared to an otherwise comparable composition lacking the L-Methionine as determined by size exclusion chromatography at 40° C. In embodiments, the rate of aggregation is from 0.5% to 1% per month as determined by size exclusion chromatography at 40° C. In embodiments, a level of oxidation is reduced as compared to an otherwise comparable composition lacking the L-Methionine as determined by peptide mapping of W102 or W104 of SEQ ID NO: 1 of the anti-ILT7 binding protein at room temperature. In embodiments, when a composition is exposed to light at 1600-1800 lux for 5 days, the percentage of oxidized species per day is from 0.2% to 0.5%. In embodiments, a composition is Fc oxidation resistant.

Provided are containers comprising a composition of the disclosure in unit dose form. In embodiments, the container comprises nitrogen. In embodiments, the container does not comprise nitrogen.

Provided are methods of treating a disease comprising administering an effective amount of the composition of the disclosure to a subject in need thereof, thereby treating the disease. In embodiments, the administering is sufficient to reduce a symptom of the disease in the subject in need thereof after the administering. In embodiments, the symptom is reduced by at least about 1-fold.

Provided are methods of treating an autoimmune disease comprising administering an effective amount of a composition of the disclosure to a subject in need thereof, thereby treating the autoimmune disease. In embodiments, the autoimmune disease is selected from the group consisting of: Discoid lupus erythematosus (DLE), focal segmental glomerulosclerosis (FSGS), alopecia areata, myositis, diabetes, Hashimoto's disease, autoimmune adrenal insufficiency, pure red cell anemia, multiple sclerosis, rheumatoid carditis, lupus nephritis, systemic lupus erythematosus, psoriasis, rheumatoid arthritis, chronic inflammation, Sjögren's syndrome, polymyositis, dermatomyositis, inclusion body myositis, juvenile myositis, IgG4 related disease (IgG4RD), and scleroderma. In embodiments, the autoimmune disease is alopecia areata. In embodiments, the autoimmune disease is systemic lupus erythematosus. In embodiments, the autoimmune disease is FSGS.

Provided are methods of treating alopecia areata comprising administering an effective amount of a composition of the disclosure to a subject in need thereof, thereby treating the alopecia areata.

Provided are methods of treating FSGS comprising administering an effective amount of a composition of the disclosure to a subject in need thereof, thereby treating the FSGS.

Provided are methods of treating systemic lupus erythematosus comprising administering an effective amount of a composition of the disclosure to a subject in need thereof, thereby treating the systemic lupus erythematosus.

In embodiments, compositions of the disclosure are administered every 4 weeks. In embodiments, compositions of the disclosure are administered every 12 weeks. In embodiments, the administrations continue for at least about 2 administrations.

Provided are methods of treating myositis comprising administering an effective amount of a composition of the disclosure to a subject in need thereof, thereby treating the myositis. In embodiments, administration of compositions of the disclosure are subcutaneous.

Provided are compositions, comprising about: i. 150 mg/mL of an immunoglobulin-like transcript 7 (ILT7) binding protein; ii. 20 mM histidine/histidine-HCl; iii. 180 mM Sucrose; iv. 15 mM L-Methionine; and v. 0.02% Polysorbate-80, wherein the anti-ILT7 binding protein comprises a sequence having at least 85% identity to a sequence of Table 1.

Provided are compositions, comprising about: i. 150 mg/mL of an immunoglobulin-like transcript 7 (ILT7) binding protein; ii. 20 mM histidine/histidine-HCl; iii. 180 mM Sucrose; iv. 15 mM L-Methionine; and v. 0.02% Polysorbate-80, wherein the anti-ILT7 binding protein comprises a VH comprising SEQ ID NO: 1, a VL comprising SEQ ID NO: 2, or both the VH and VL of SEQ ID NO: 1 and SEQ ID NO: 2, respectively.

Provided are methods of treating an autoimmune disease comprising administering a composition that comprises: i. about 150 mg/mL of an immunoglobulin-like transcript 7 (ILT7) binding protein; ii. 20 mM histidine/histidine-HCl; iii. 180 mM Sucrose; iv. 15 mM L-Methionine; and v. 0.02% Polysorbate-80, wherein the anti-ILT7 binding protein comprises Complementarity-Determining Regions (CDRs) HCDR1, HDR2, HCDR3, LCDR1, LCDR2, and LCDR3 comprising the sequences of SEQ ID NOs: 3, 4, 5, 6, 7, and 8 respectively, wherein the dose of the anti-ILT7 binding protein comprises from about 150 mg to about 450 mg; wherein the administration is subcutaneous, and wherein the dose is administered in a single injection. In embodiments, the dose of the anti-ILT7 binding protein comprises from about 150 mg to about 275 mg. In embodiments, the dose of the anti-ILT7 binding protein comprises from about 150 mg to about 200 mg.

Provided are delivery devices comprising any of the compositions disclosed herein. In embodiments, a delivery device is an autoinjector.

Provided are kits comprising a delivery device disclosed herein, and instructions for use thereof. In embodiments, a kit comprises a plurality of autoinjectors.

Provided are methods of treatment, comprising administering daxdilimab to a subject in need thereof, wherein the administering comprises an autoinjector. In embodiments, an autoinjector of the disclosure is contacted with the thigh, abdomen, or arm of a subject in need thereof in a manner sufficient to effectuate administration of the daxdilimab the subject in need.

Provided are methods of treating an autoimmune disease, wherein the treatment is of an autoimmune disease or condition selected from the group consisting of: Discoid lupus erythematosus (DLE), Focal segmental glomerulosclerosis (FSGS), alopecia areata, lupus nephritis, myositis, diabetes, Hashimoto's disease, autoimmune adrenal insufficiency, pure red cell anemia, multiple sclerosis, rheumatoid carditis, systemic lupus erythematosus, psoriasis, rheumatoid arthritis, chronic inflammation, Sjogren's syndrome, polymyositis, dermatomyositis, inclusion body myositis, juvenile myositis, IgG4 related disease (IgG4RD), and scleroderma. In embodiments, a method disclosed herein provides treatment of a non-autoimmune disease or condition. In embodiments, a non-autoimmune disease or condition comprises an ILT7-expressing cell-mediated disease.

Provided are compositions, comprising (i) an immunoglobulin-like transcript 7 (ILT7) binding protein; and (ii) L-methionine; wherein when the composition is exposed to light at 1600-1800 lux for 5 days, the increase in percentage of oxidized species of ILT7 binding protein in the composition is at most about 1.0%. In embodiments, an increase in the percentage of oxidized species of ILT7 binding protein in the composition is at most about 0.4%, 0.3%, 0.2%, or 0.1%.

Provided are compositions, comprising (i) an immunoglobulin-like transcript 7 (ILT7) binding protein; and (ii) L-methionine; wherein when the composition is exposed to light at 1600-1800 lux the rate of formation of new oxidized species per day of ILT7 binding protein in the composition is at most about 0.1%-1.0%. In embodiments, a rate of formation of new oxidized species per day of the ILT7 binding protein in the composition is at most about 0.2%-0.4% or 0.1%-0.3%.

Provided are compositions, comprising about 100 mg/mL to about 450 mg/mL of an ILT7 binding protein. In embodiments, an ILT7 binding proteins comprises Complementarity-Determining Regions (CDRs) HCDR1, HDR2, HCDR3, LCDR1, LCDR2, and LCDR3 comprising the sequences of SEQ ID NOs: 3, 4, 5, 6, 7, and 8, respectively.

These and other embodiments are described herein.

### BRIEF DESCRIPTION OF THE DRAWINGS

The accompanying drawings, which are incorporated herein and form a part of the specification, illustrate some, but not the only or exclusive, example embodiments and/or features. It is intended that the embodiments and figures disclosed herein are to be considered illustrative rather than limiting.

FIGS. 1A-1C shows a schematic of an exemplary afucosylated human IgG12 anti-ILT7 mAb “anti-ILT7 mAb” (FIG. 1A) and percentage of oxidized species for formulations containing 10 mM excipient L-methionine (L-Met) as an excipient or controls (0 mM L-Met) in addition to 100 mg/mL anti-ILT7 mAb, 20 mM histidine/histidine-HCl, 240 mM sucrose, and 0.02% PS-80 at pH 6.0. Oxidized species were assessed at 5° C. (FIG. 1B), and RT (FIG. 1C). Results show that L-Met reduced the rate of oxidized species. RT=room temperature.

FIGS. 2A-2C show percentage of aggregates for formulations containing 10 mM excipient L-methionine (L-Met) as an excipient or controls (0 mM L-Met) in addition to 100 mg/mL anti-ILT7 mAb, 20 mM histidine/histidine-HCl, 240 mM sucrose, and 0.02% PS-80 at pH 6.0. Aggregates were assessed at 40° C. (FIG. 2A), RT (FIG. 2B), and 5° C. (FIG. 2C). Results show that L-Met reduced aggregation rate of the anti-ILT7 mAb. RT=room temperature.

FIGS. 3A-3B show percentage rate per month of oxidation (FIG. 3A) and aggregation (FIG. 3B) for formulations containing L-Met (0 mM, 5 mM, 10 mM, and 20 mM) in addition to 150 mg/mL anti-ILT7 mAb, 20 mM histidine/histidine-HCl, 200 mM sucrose, and 0.02% PS-80 at pH 6.0. Percent formation was measured at 5° C., RT, and 40° C. 15 mM L-Met resulted in suitable stabilization and robustness. RT=room temperature.

FIGS. 4A-4B show the percentage of oxidized species formed per day for formulations containing 100 mg/mL anti-ILT7 mAb, 20 mM histidine/histidine-HCl, 240 mM sucrose, 0.02% PS-80 at pH 6.0 (P2) or 150 mg/mL anti-ILT7 mAb, 20 mM histidine/histidine-HCl, 200 mM sucrose, and 0.02% PS-80 at pH 6.0 (P3) exposed to light (FIG. 4A) and dark (FIG. 4B). The effect of the presence of air, nitrogen (N2), or 15 mM L-Met on oxidized species formation in formulations in light or dark conditions was assessed. Vials were held in light box at ambient room temperature and light intensity was measured as 1600-1800 lux throughout experiment. 15 mM L-Met minimized oxidized species formation when exposed to light for 5 days.

FIGS. 5A-5C show the % oxidized variants formation as a function of time for formulations containing 10 mg/mL, 25 mg/mL, 50 mg/mL, 75 mg/mL, or 100 mg/mL anti-ILT6 mAb at 40° C. (FIG. 5A), RT (FIG. 5B), and 5° C. (FIG. 5C). Change in % oxidized variants formation at 40° C. was measured at approximately 0, 0.25, 0.5, 1, 2, and 3 months. Change in % oxidized variants formation at RT was measured at approximately 0, 1, 2, 3, 6, 8, and 12 months. Change in % oxidized variants formation at 5° C. was measured at approximately 0, 3, 6, 9, and 12, months. % oxidized variants formation was measured using size exclusion chromatography (SEC). RT=room temperature.

FIGS. 6A-6C show the change in % monomer as a function of time, container type (pre-filled syringe [PFS] or vial), and volume (1 mL PFS or 2 mL PFS) at 40° C. (FIG. 6A), 25° C. (FIG. 6B), and 5° C. (FIG. 6C). Change in % monomer at 40° C. was measured at approximately 0, 0.5, 1, 2, and 3 months. Change in % monomer at 25° C. was measured at approximately 0, 1, 2, 3, and 6 months. Change in % monomer at 5° C. was measured at approximately 0, 3, 6, 9, 12, 16, and 20 months. % monomer was measured using size exclusion chromatography (SEC).

FIGS. 7A-7C show the % oxidized variants formation as a function of time, container type (pre-filled syringe [PFS] or vial) and volume (1 mL PFS or 2 mL PFS) at 40° C. (FIG. 7A), 25° C. (FIG. 7B), and 5° C. (FIG. 7C). Change in % oxidized variants formation at 40° C. was measured at approximately 0, 0.5, 1, 2, and 3 months. Change in % oxidized variants formation at 25° C. was measured at approximately 0, 1, 2, 3, and 6 months. Change in % oxidized variants formation at 5° C. was measured at approximately 0, 3, 6, 9, 12, 16, and 20 months. % oxidized variants formation was measured using size exclusion chromatography (SEC).

### DETAILED DESCRIPTION

Provided are compositions containing anti-ILT7 binding proteins that have increased potency and shelf-life. Also provided are methods of making and using the same in the treatment of disease including but not limited to autoimmune indications. The compositions find particular use in improving stability of antibodies having the sequence of SEQ ID NO: 1. Amino acid residues on this antibody are compromised by light and oxygen. In particular, described are tryptophan residues that become oxidized, asparagine residues that are deamidated, and portions for the Fc region that are oxidized. The disclosed compositions are particularly beneficial with respect to reducing oxidation and aggregation. Also provided are devices (e.g., autoinjectors) that comprise the disclosed compositions and methods of utilizing the same.

In embodiments, an anti-ILT7 mAb containing compositions can be formulated to reduce or eliminate light-induced tryptophan oxidation of one or more residues in a complementary determining region of the anti-ILT7 mAb by way of inclusion of L-methionine in the formulation. In embodiments, L-methionine further reduces aggregation levels of the anti-ILT7 mAb formulation thereby resulting in an improved formulation conferring an extended shelf-life as compared to existing formulations.

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

Any ranges listed herein are intended to be inclusive of endpoints. For example, a range of 2-4 includes 2 and 4 and values between.

As used herein, the singular forms “a,” “an,” and “the” include plural referents unless the content clearly dictates otherwise.

The term “about” or “approximately” when immediately preceding a numerical value means a range plus or minus 10% of that value. For example, “about 50” can mean 45 to 55, “about 25,000” can mean 22,500 to 27,500, etc., unless the context of the disclosure indicates otherwise, or is inconsistent with such an interpretation. In the context of a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

When referring to a nucleic acid sequence or amino acid sequence, the term “identity” is used to denote similarity between two sequences. Unless otherwise indicated, percent identities described herein are determined using the BLAST algorithm available at the world wide web address: blast.ncbi.nlm.nih.gov/Blast.cgi using default parameters.

As used herein, the term “subject” refers to any individual, e.g., a human or a non-human mammal, for whom diagnosis, prognosis, or therapy is desired. The term “subject” may mean a human or non-human mammal affected, likely to be affected, or suspected to be affected with a disease, e.g., an autoimmune disease or condition. The terms “subject” and “patient” are used interchangeably herein. Although the anti-ILT7 binding protein compositions provided herein are principally directed to compositions which are suitable for administration to humans, the skilled artisan will understand that such compositions are generally suitable for administration to subjects of all sorts. In embodiments, the subject is a mammal. A mammal includes primates, such as humans, monkeys, chimpanzee, and apes, and non-primates such as domestic animals, including laboratory animals (such as rabbits and rodents, e.g., guinea pig, rat, or mouse) and household pets and farm animals (e.g., cats, dogs, swine, cattle, sheep, goats, horses, rabbits), and non-domestic animals, such as wildlife, birds, reptile, fish, and the like.

As used herein the term “room temperature” or “RT” refers to a temperature of about 20° C. to about 25° C. RT can be 21° C., 22° C., 23° C., 24° C., or up to about 25° C. RT can be from about 20° C.-21° C., 21° C.-22° C., 22° C.-23° C., 23° C.-24° C., or 24° C.-25° C.

As used herein, “treating” or “treat” describes the management and care of a subject for the purpose of combating a disease, condition, or disorder and includes the administration of an ILT7-binding protein used in the methods described herein to alleviate the symptoms or complications of a disease, condition, or disorder, or to eliminate the disease, condition, or disorder. Thus, the term “treat” or “treating” refers to both therapeutic measures and prophylactic or preventative measures, wherein the objective is to prevent, slow down (lessen), or ameliorate the progression of a disease (e.g., an autoimmune disease). Beneficial or desired clinical results include, but are not limited to, alleviation of symptoms, diminishing the extent of the disease, stabilized (i.e., not worsening) state of the disease, delaying or slowing of disease progression, amelioration, or palliation of the disease state, and reversing the disease (whether partial or total). The term “treat” can also include treatment of a cell in vitro or an animal model.

Generally, the terms “ILT7 binding protein” and “ILT7 binding molecule,” are used interchangeably to refer to a protein or molecule that binds to immunoglobulin-like transcript 7 (ILT7). The terms protein and peptide can be used interchangeably herein. In embodiments, the anti-ILT7 binding proteins used in the methods described herein bind to full-length ILT7. In embodiments, the anti-ILT7-binding proteins used in the methods described herein bind to a fragment of ILT7. In embodiments, the fragment of ILT7 to which the ILT7 binding proteins bind comprises the extracellular domain of ILT7. In embodiments, any of an ILT7 binding protein or an ILT7 binding molecule can refer to an anti-ILT7 binding protein (e.g., an antibody).

### Compositions Comprising an Anti-ILT7 Binding Protein

### Immunoglobulin-Like Transcript 7 (ILT7) Binding Protein

In embodiments, provided is an anti-ILT7 binding protein. In embodiments, the anti-ILT7 binding protein binds to any mammalian ILT7. In embodiments, the anti-ILT7 binding protein binds to human ILT7 or a fragment thereof, for example the extracellular portion of human ILT7. In embodiments, the anti-ILT7 binding protein disclosed herein binds to cynomolgus ILT7 or a fragment thereof, for example the extracellular portion of cynomolgus ILT7.

Exemplary sequences for anti-ILT7 binding proteins are disclosed and described in PCT Publication No. WO2017156298, U.S. Pat. No. 8,084,585, PCT Publication No. WO2021113702 all of which are incorporated by reference herein in their entirety. Exemplary sequences for an anti-ILT7 binding protein is shown in Table 1. In embodiments, the ILT7 to which the anti-ILT7 binding protein binds is located on plasmacytoid dendritic cells (pDCs). In embodiments, clone ILT70137 in PCT Publication No. WO 2017/156298 is provided and further described below in Table 1.

In embodiments, any one of the sequences from Table 1 can be modified. In embodiments, a modification to the sequences of Table 1 comprises one or more truncations, deletions, insertions, substitutions, and any combination of modification thereof. A modification can occur at any of the residues provided in Table 1 and in any number of residues from Table 1. In embodiments, a modification can comprise from 1-3, 1-5, 1-10, 5-20, 1-3, 1-5, 1-10, 1-20, 3-8, 3-10, 3-15, 5-8, 5-10, or 5-20 residues. In embodiments, a modification can occur in up to 1, 2, 3, 4, 5, 6, 7, 8, 9, 10, 11, 12, 13, 14, 15, 20, 30, 40, 50 residues.

TABLE 1
Exemplary Anti-ILT7 Binding Protein Sequences

| SEQ | | |
|---|---|---|
| ID NO. | Description | Sequence |
| 1 | Heavy chain | QVQLQQSGAEVKKPGASVKVSCKASGYTFTSYGISWVRQ |
| | variable region | APGQGLEWMGWISAYNGNTNYAQKLQGRVTMTTDTSTST |
| | (VH) | AYMELRSLRSDDTAVYYCARNGLWGWDSDAFDIWGRGT |
| | | LVTVSS |
| 2 | Light chain | QSALTQPASVSGSPGQSITISCTGTSSDVGGYNYVSWYQQH |
| | variable region | PGKAPKLMIYDVSNRPSGVSNRFSGSKSGNTASLTISGLQA |
| | (VL) | EDEADYYCSSYTSSSTVVFGGGTKVTVL |
| 3 | HCDR1 | SYGIS |
| 4 | HCDR2 | WISAYNGNTNYAQKLQG |
| 5 | HCDR3 | NGLWGWDSDAFDI |
| 6 | LCDR1 | TGTSSDVGGYNYVS |
| 7 | LCDR2 | DVSNRPS |
| 8 | LCDR3 | SSYTSSSTVV |
| 9 | LC | QSALTQPASVSGSPGQSITISCTGTSSDVGGYNYVSWYQQH |
| | | PGKAPKLMIYDVSNRPSGVSNRFSGSKSGNTASLTISGLQA |
| | | EDEADYYCSSYTSSSTVVFGGGTKVTVLGQPKAAPSVTLFP |
| | | PSSEELQANKATLVCLISDFYPGAVTVAWKADSSPVKAGV |
| | | ETTTPSKQSNNKYAASSYLSLTPEQWKSHRSYSCQVTHEGS |
| | | TVEKTVAPTECS |
| 10 | HC | QVQLQQSGAEVKKPGASVKVSCKASGYTFTSYGISWVRQ |
| | | APGQGLEWMGWISAYNGNTNYAQKLQGRVTMTTDTSTST |
| | | AYMELRSLRSDDTAVYYCARNGLWGWDSDAFDIWGRGT |
| | | LVTVSSASTKGPSVFPLAPSSKSTSGGTAALGCLVKDYFPEP |
| | | VTVSWNSGALTSGVHTFPAVLQSSGLYSLSSVVTVPSSSLG |
| | | TQTYICNVNHKPSNTKVDKRVEPKSCDKTHTCPPCPAPELL |
| | | GGPSVFLFPPKPKDTLMISRTPEVTCVVVDVSHEDPEVKEN |
| | | WYVDGVEVHNAKTKPREEQYNSTYRVVSVLTVLHQDWL |
| | | NGKEYKCKVSNKALPAPIEKTISKAKGQPREPQVYTLPPSR |
| | | EEMTKNQVSLTCLVKGFYPSDIAVEWESNGQPENNYKTTP |
| | | PVLDSDGSFFLYSKLTVDKSRWQQGNVFSCSVMHEALHN |
| | | HYTQKSLSLSPGK |
| 11 | Polynucleotide | caggtacagctgcagcagtcaggagctgaggtgaagaagcctggggcctcagtgaaggtct |
| | encoding VH | cctgcaaggcttctggttacacctttaccagctatggtatcagctgggtgcgacaggcccctgg |
| | | acaagggcttgagtggatgggatggatcagcgcttacaatggtaacacaaactatgcacaga |
| | | agctccagggcagagtcaccatgaccacagacacatccacgagcacagcctacatggagct |
| | | gaggagcctgagatctgacgacacggccgtgtattactgtgcgagaaatggcctctgggggt |
| | | gggactctgatgcttttgatatctggggccgaggaaccctggtcaccgtctcctca |
| 12 | Polynucleotide | cagtctgccctgactcagcctgcctccgtgtctgggtctcctggacagtcgatcaccatctcct |
| | encoding VL | gcactggaaccagcagtgacgttggtggttataactatgtctcctggtaccaacagcacccag |
| | | gcaaagcccccaaactcatgatttatgatgtcagtaatcggccctcaggggtttctaatcgcttc |
| | | tctggctccaagtctggcaacacggcctccctgaccatctctgggctccaggctgaggacga |
| | | ggctgattattactgcagctcatatacaagcagcagcactgtggtattcggcggagggaccaa |
| | | ggtcaccgtccta |

Provided herein are compositions that comprise an anti-ILT7 binding protein. In embodiments, a composition may comprise an effective amount of an anti-ILT7 binding protein. In embodiments, the anti-ILT7 binding protein or a polynucleotide encoding at least a portion of the protein comprises at least about or at most about 50%, 55%, 60%, 65%, 70%, 75%, 80%, 85%, 90%, 95%, 96%, 97%, 98%, 99%, or up to about 100% identity to any one of the sequences of Table 1 (SEQ ID NOs: 1-12). In embodiments, the anti-ILT7 binding protein or a polynucleotide encoding at least a portion of the protein comprises any one of the sequences of Table 1.

In embodiments, an isolated ILT7 binding protein is an anti-ILT7 binding protein that can bind to the same ILT7 epitope as an antibody comprising a heavy chain variable region (VH) of SEQ ID NO: 1 and a light chain variable region (VL) of SEQ ID NO: 2. In embodiments, an isolated ILT7 binding protein is an anti-ILT7 binding protein that competitively inhibits the binding to ILT7 of an antibody comprising a VH of SEQ ID NO: 1 and a VL of SEQ ID NO: 2.

In embodiments, an isolated ILT7 binding protein is an anti-ILT7 binding protein comprising Complementarity-Determining Regions (CDRs) HCDR1, HDR2, HCDR3, LCDR1, LCDR2, and LCDR3 comprising the sequences of SEQ ID NOs: 3, 4, 5, 6, 7, and 8, respectively.

In embodiments, an anti-ILT7 binding protein comprises a VH at least 80%, 85%, 90%, 95%, 96%, 97%, 98% or 99% identical to SEQ ID NO: 1 and/or a VL at least 80%, 85%, 90%, 95%, 96%, 97%, 98% or 99% identical to SEQ ID NO: 2.

In embodiments, an anti-ILT7 binding protein comprises a VH comprising SEQ ID NO: 1 and a VL comprising SEQ ID NO: 2. In embodiments, an isolated ILT7 binding protein is an anti-ILT7 binding protein comprising a VH comprising SEQ ID NO: 1. In embodiments, an isolated ILT7 binding protein is an anti-ILT7 binding protein comprising a VH comprising SEQ ID NO: 2.

In embodiments, an ILT7 binding protein comprises a light chain (LC) and a heavy chain (HC) at least 85%, 90%, 95%, 96%, 97%, 98% or 99% identical to: SEQ ID NO: 9 and SEQ ID NO: 10, respectively. In embodiments, an ILT7 binding protein comprises a LC that comprises SEQ ID NO: 9, with 1, 2, 3, 5, 6, 7, 8, 9, 10 or up to 20 residue modifications. In embodiments, an ILT7 binding protein comprises a HC that comprises SEQ ID NO: 10, with 1, 2, 3, 5, 6, 7, 8, 9, 10 or up to 20 residue modifications. In embodiments, an ILT7 binding protein comprises a LC that comprises SEQ ID NO: 9. In embodiments, an ILT7 binding protein comprises a HC that comprises SEQ ID NO: 10.

In embodiments, a composition of the disclosure can comprise a residue modification, e.g., a post-translational modification (PTM). A modification described herein comprises a PTM. Exemplary PTMs can occur on an amino acid side chain or at a protein's C- or N-termini. In embodiments, a modification comprises at least a portion of a CDR region. In embodiments, a modification comprises at least a portion of a fragment crystallizable (Fc) region. In embodiments, a modification is reversible. In embodiments, a modification is irreversible. Exemplary modifications comprise phosphorylation, glycosylation, ubiquitination, nitrosylation, methylation, acetylation, lipidation and proteolysis.

In embodiments, an ILT7 binding protein comprises an antibody or antigen-binding fragment thereof. In embodiments, the antibody or antigen-binding fragment thereof is afucosylated.

In embodiments, an ILT7 binding protein binds to the Ig1 region of ILT7. In embodiments, an ILT7 binding protein binds to the Ig2 region of ILT7. In embodiments, an ILT7 binding protein binds to human and cynomolgus ILT7.

In embodiments, an ILT7 binding protein suppresses interferon (IFN) alpha release from peripheral blood mononuclear cells (PBMCs). In embodiments, an ILT7 binding protein has ADCC activity against plasmacytoid dendritic cells (pDCs) in PMBCs.

In embodiments, an ILT7 binding protein comprises a murine, human, chimeric, humanized, or resurfaced antibody or antigen-binding fragment thereof. In embodiments, an anti-ILT7 binding protein comprises an antibody, Fab, Fab′, F(ab′)2, Fd, single chain Fv or scFv, disulfide linked Fv, V-NAR domain, IgNar, intrabody, IgGΔCH2, minibody, F(ab′)3, tetrabody, triabody, diabody, single-domain antibody, DVD-Ig, Fcab, mAb2, (scFv)2, or scFv-Fc. In embodiments, an ILT7 binding protein comprises a monoclonal antibody or an antigen binding fragment thereof.

In embodiments, an ILT7 binding protein comprises a HC immunoglobulin constant domain selected from the group consisting of: (a) an IgA constant domain; (b) an IgD constant domain; (c) an IgE constant domain; (d) an IgG1 constant domain; (e) an IgG2 constant domain; (f) an IgG3 constant domain; (g) an IgG4 constant domain; and (h) an IgM constant domain. In embodiments, an anti-ILT7 binding protein comprises a LC immunoglobulin constant domain selected from the group consisting of: (a) an Ig kappa constant domain; and (b) an Ig lambda constant domain. In embodiments, an anti-ILT7 binding protein comprises a human IgG1 constant domain and a human lambda constant domain.

In embodiments, provided herein is a host cell producing an anti-ILT7 binding molecule.

In embodiments, provided herein is an isolated polynucleotide comprising a nucleic acid encoding a VH, wherein the VH comprises an amino acid sequence at least 85%, 90%, 95% identical, or identical to the VH of SEQ ID NO: 1. In embodiments, the polynucleotide comprises a sequence at least 85%, 90%, 95% identical to SEQ ID NO: 11. In embodiments, the polynucleotide comprises SEQ ID NO: 11.

In embodiments, provided herein is an isolated polynucleotide comprising a nucleic acid encoding a VL, wherein the VL comprises an amino acid sequence at least 85%, 90%, 95% identical, or identical to the VL of SEQ ID NO: 2. In embodiments, the polynucleotide comprises a sequence at least 85%, 90%, 95% identical to SEQ ID NO: 12. In embodiments, the polynucleotide comprises SEQ ID NO: 12.

In embodiments, the nucleic acid is operably linked to a control sequence. In embodiments, an antibody or antigen-binding fragment thereof comprising the VH or the VL encoded by the nucleic acid can specifically bind to ILT7.

In embodiments, a polynucleotide encodes an anti-ILT7 binding molecule provided herein.

In embodiments, provided herein is a vector comprising the polynucleotide.

In embodiments, provided herein is a polypeptide encoded by the polynucleotide.

In embodiments, provided herein is a host cell transformed with a polynucleotide provided herein (e.g., a polynucleotide comprising a nucleic acid encoding a VH and a polynucleotide comprising a nucleic acid encoding a VL).

In embodiments, provided herein is a host cell comprising a polynucleotide provided herein (e.g., a polynucleotide comprising a nucleic acid encoding a VH and a polynucleotide comprising a nucleic acid encoding a VL), a vector provided herein, or a polypeptide provided herein. In embodiments, the host cell is a mammalian host cell. In embodiments, the host cell is a NS0 murine myeloma cell, a PER.C6R human cell, or a Chinese hamster ovary (CHO) cells. In embodiments, the host cell lacks the enzyme α-1,6-fucosyltransferase.

In embodiments, a composition of the disclosure may comprise from about, at least about, or at most about: 100 mg/mL, 102 mg/mL, 104 mg/mL, 106 mg/mL, 108 mg/mL, 110 mg/mL, 112 mg/mL, 114 mg/mL, 116 mg/mL, 118 mg/mL, 120 mg/mL, 122 mg/mL, 124 mg/mL, 126 mg/mL, 128 mg/mL, 130 mg/mL, 132 mg/mL, 134 mg/mL, 136 mg/mL, 138 mg/mL, 140 mg/mL, 142 mg/mL, 144 mg/mL, 146 mg/mL, 148 mg/mL, 150 mg/mL, 155 mg/mL, 165 mg/mL, 175 mg/mL, 185 mg/mL, 195 mg/mL, 205 mg/mL, 215 mg/mL, 225 mg/mL, 235 mg/mL, 245 mg/mL, 250 mg/mL, 300 mg/mL, 350 mg/mL, 375 mg/mL, 400 mg/mL, or up to about 450 mg/mL of an anti-ILT7 binding protein.

In embodiments, a composition may comprise from about 100 mg/mL to about 350 mg/mL of an anti-ILT7 binding protein. In embodiments, a composition may comprise from about 150 mg/mL to about 275 mg/mL of an anti-ILT7 binding protein. In embodiments, a composition may comprise from about 200 mg/mL to about 250 mg/mL of an anti-ILT7 binding protein. In embodiments, a composition may comprise from about 100 mg/mL to about 150 mg/mL of an anti-ILT7 binding protein. In embodiments, a composition may comprise from about 110 mg/mL to about 150 mg/mL of an anti-ILT7 binding protein. In embodiments, a composition may comprise from about 120 mg/mL to about 150 mg/mL of an anti-ILT7 binding protein. In embodiments, a composition may comprise from about 130 mg/mL to about 150 mg/mL of an anti-ILT7 binding protein.

### Sugar

In embodiments, a composition of the disclosure comprises an anti-ILT7 binding protein and a carrier. In embodiments, a carrier is a stabilizer. In embodiments, a stabilizer is a sugar. In embodiments, a composition provided herein comprises a sugar. In embodiments, sugars for use with the compositions of the disclosure include, but are not limited to, monosaccharides, disaccharides and trisaccharides, or combinations thereof. In embodiments, a composition comprises a sugar, wherein the sugar may be e.g., sucrose, trehalose, raffinose, maltose, sorbitol or mannitol, or combinations thereof. In embodiments, the sugar may be a sugar alcohol or an amino sugar. In embodiments, a sugar is sucrose.

In embodiments, a composition that comprises an anti-ILT7 binding protein comprises a sugar. Any amount of a sugar may be utilized. In embodiments, from about, at least about, or at most about: 150 nM to about 240 mM sugar is utilized. In embodiments, a composition comprises from about 160 nM to about 240 mM sugar. In embodiments, a composition comprises from about 170 nM to about 240 mM sugar. In embodiments, a composition comprises from about 180 nM to about 240 mM sugar. In embodiments, a composition comprises from about 190 nM to about 240 mM sugar. In embodiments, a composition comprises from about 200 nM to about 240 mM sugar. In embodiments, a composition comprises from about 210 nM to about 240 mM sugar. In embodiments, a composition comprises from about 220 nM to about 240 mM sugar. In embodiments, a composition comprises from about 230 nM to about 240 mM sugar. In embodiments, a composition comprises about 150 nM to about 200 mM sugar. In embodiments, a composition comprises about 170 nM to about 190 mM of a sugar.

In embodiments, a composition that comprises an anti-ILT7 binding protein comprises a sugar. Any amount of a sugar may be utilized. In embodiments, from about, at least about, or at most about: 150 mM to about 240 mM sugar is utilized. In embodiments, a composition comprises from about 160 mM to about 240 mM sugar. In embodiments, a composition comprises from about 170 mM to about 240 mM sugar. In embodiments, a composition comprises from about 180 mM to about 240 mM sugar. In embodiments, a composition comprises from about 190 mM to about 240 mM sugar. In embodiments, a composition comprises from about 200 mM to about 240 mM sugar. In embodiments, a composition comprises from about 210 mM to about 240 mM sugar. In embodiments, a composition comprises from about 220 mM to about 240 mM sugar. In embodiments, a composition comprises from about 230 mM to about 240 mM sugar. In embodiments, a composition comprises about 150 mM to about 200 mM sugar. In embodiments, a composition comprises about 170 mM to about 190 mM of a sugar.

In embodiments, a composition that comprises an anti-ILT7 binding protein comprises sucrose. In embodiments, a compositions may comprise any amount of sucrose. In embodiments, a compositions may comprise an amount of sucrose from about, at least about, or at most about: 150 nM to about 240 mM. In embodiments, a composition comprises from about 160 nM to about 240 mM sucrose. In embodiments, a composition comprises from about 170 nM to about 240 mM sucrose. In embodiments, a composition comprises from about 180 nM to about 240 mM sucrose. In embodiments, a composition comprises from about 190 nM to about 240 mM sucrose. In embodiments, a composition comprises from about 200 nM to about 240 mM sucrose. In embodiments, a composition comprises from about 210 nM to about 240 mM sucrose. In embodiments, a composition comprises from about 220 nM to about 240 mM sucrose. In embodiments, a composition comprises from about 230 nM to about 240 mM sucrose. In embodiments, a composition comprises about 150 nM to about 200 mM sucrose. In embodiments, a composition comprises about 170 nM to about 190 mM sucrose.

In embodiments, a composition that comprises an anti-ILT7 binding protein comprises from about, at least about, or at most about: 150 mM, 155 mM, 160 mM, 165 mM, 170 mM, 175 mM, 180 mM, 185 mM, 190 mM, 195 mM, 200 mM, 205 mM, 210 mM, 215 mM, 220 mM, 225 mM, 230 mM, 235 mM, 240 mM, 245 mM, 250 mM of a sugar (e.g., sucrose). In embodiments, a composition comprises about 180 mM sucrose.

### Polysorbate-80

In embodiments, a composition of the disclosure comprises an anti-ILT7 binding protein and a carrier. In embodiments, a carrier is a stabilizer. In embodiments, a stabilizer is polysorbate-80 (PS-80). In embodiments, a composition comprises an anti-ILT7 binding protein and PS-80. In embodiments, a composition comprises from about, at least about, or at most about: 0.002%, 0.008%, 0.01%, 0.02%, 0.022%, 0.042%, 0.062%, 0.082%, 0.102%, 0.122%, 0.142%, 0.162%, 0.182%, 0.202%, 0.222%, 0.242%, 0.262%, 0.282%, 0.302%, 0.322%, 0.342%, 0.362%, 0.382%, 0.402%, 0.422%, 0.442%, 0.462%, 0.482%, 0.502%, 0.522%, 0.542%, 0.562%, 0.582%, 0.602%, 0.622%, 0.642%, 0.662%, 0.682%, 0.702%, 0.722%, 0.742%, 0.762%, 0.782%, or up to 0.8% PS-80. In embodiments, a composition comprises from about 0.002% to about 0.8% polysorbate-80. In embodiments, a composition comprises about 0.01% to about 0.8% PS-80. In embodiments, a composition comprises from about 0.01% to about 0.3% PS-80. In embodiments, a composition comprises from about 0.01% to about 0.05% PS-80. In embodiments, a composition provided herein comprises about 0.02% PS-80.

In embodiments, about 0.02% PS-80 is effective in reducing particle formation under freeze/thaw (F/T) (e.g., 30° C./exposure to temperature of greater than about 30° C.), agitation stress, or both F/T and agitation stress of a composition provided herein. In embodiments, about 0.02% PS-80 is effective in reducing particle formation under F/T, agitation stress, or both F/T and agitation stress of a composition provided herein as compared to an otherwise comparable composition lacking the PS-80 or lacking the PS-80 at about 0.02%. In embodiments, about 0.02% PS-80 is effective in reducing particle formation under F/T, agitation stress, or both F/T and agitation stress of a composition provided herein as compared to an otherwise comparable composition lacking the PS-80 or lacking the PS-80 at about 0.02% by at least about 1-fold, 5-fold, 10-fold, 30-fold, 50-fold, 100-fold, 150-fold, or 300-fold. In embodiments, about 0.02% PS-80 is effective in reducing particle formation under F/T, agitation stress, or both F/T and agitation stress of a composition provided herein as compared to an otherwise comparable composition lacking the PS-80 or lacking the PS-80 at about 0.02% by at least about 3%, 10%, 20%, 40%, 60%, 80%, 100%, 150%, 200%, or about 300%.

### Methionine

In embodiments, a composition of the disclosure comprises an anti-ILT7 binding protein and a carrier. In embodiments, a carrier is a stabilizer. In embodiments, a stabilizer is an amino acid (e.g., methionine). In embodiments, a composition comprises an anti-ILT7 binding protein and methionine. In embodiments, the methionine is L-methionine. L-methionine is the L-enantiomer of methionine. In embodiments, a composition provided herein comprises L-methionine (i.e. (S)-2-Amino-4-(methylmercapto) butyric acid, L-2-Amino-4-(methylthio) butanoic acid). In embodiments, the methionine is D-methionine. In embodiments, a composition comprises L-methionine and D-methionine. In embodiments, a composition comprises an anti-ILT7 binding protein and L-methionine.

Any concentration of methionine can be utilized in a composition. In embodiments, a composition that comprises an anti-ILT7 binding protein comprises from about, at least about, or at most about 5 nM to about 30 mM L-methionine. In embodiments, a composition that comprises an anti-ILT7 binding protein comprises from about, at least about, or at most about 1 nM to about 25 mM L-methionine. In embodiments, a composition that comprises an anti-ILT7 binding protein comprises from about, at least about, or at most about 15 nM to about 30 mM L-methionine. In embodiments, a composition that comprises an anti-ILT7 binding protein comprises from about, at least about, or at most about 20 nM to about 25 mM L-methionine. In embodiments, a composition that comprises an anti-ILT7 binding protein comprises from about, at least about, or at most about 15 nM to about 30 mM L-methionine. In embodiments, a composition that comprises an anti-ILT7 binding protein comprises from about, at least about, or at most about 10 nM to about 20 mM methionine (e.g., L-methionine).

In embodiments, a composition that comprises an anti-ILT7 binding protein comprises from about 5 mM to 10 mM, 5 mM to 20 mM, 8 mM to 15 mM, 8 mM to 20 mM, 10 mM to 20 mM, 12 mM to 25 mM, 15 mM to 30 mM, or 20 mM to 30 mM of L-methionine. In embodiments, a composition that comprises an anti-ILT7 binding protein comprises from about, at least about, or at most about: 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 35 mM, 40 mM, 45 mM, 50 mM, 55 mM, 60 mM, 65 mM, 70 mM, or 75 mM of L-methionine. In embodiments, a composition that comprises an anti-ILT7 binding protein comprises from about, at least about, or at most about 15 mM methionine (e.g., L-methionine). In embodiments, a composition that comprises an anti-ILT7 binding protein comprises from about, at least about, or at most about 15 mM L-methionine.

### Buffer

In embodiments, a composition comprised herein comprises a buffer. In embodiments, a composition provided herein comprises an anti-ILT7 binding protein and a buffer. In embodiments, a composition comprising an anti-ILT7 binding protein and a buffer can be administered to a subject. In embodiments, a composition comprising an anti-ILT7 binding protein and a buffer can be administered to a subject in a dosage form.

In embodiments, a compositions comprises a buffer, wherein the buffer may be a histidine buffer, a phosphate buffer, a citrate buffer, an alkali metal phosphate, an acetate buffer, a tris buffer, an alkali metal carbonate, or an alkaline earth metal carbonate. In embodiments, a buffer is a histidine buffer. In embodiments, a histidine buffer can comprise L-Histidine hydrochloride. In embodiments, a buffer is a phosphate buffer. In embodiments, a buffer is a citrate buffer. In embodiments, a buffer is a citrate buffer. In embodiments, a buffer does not comprise citrate or comprises reduced citrate. In embodiments, a citrate buffer is not utilized as a subject may experience injection-associated pain and/or discomfort associated with the citrate. In embodiments, a buffer does not comprise citrate.

In embodiments, a buffer provided herein can be at any concentration. In embodiments, a buffer is at a concentration from about 10 mM to 40 mM. In embodiments, a buffer is at a concentration from about 15 mM to 40 mM. In embodiments, a buffer is at a concentration from about 20 mM to 40 mM. In embodiments, a buffer is at a concentration from about 25 mM to 35 mM. In embodiments, a buffer is at a concentration from about 20 mM to 30 mM. In embodiments, a buffer is at a concentration from about 20 mM to 25 mM. In embodiments, a buffer is a histidine buffer such as histidine/histidine-HCl and is at a concentration from about 15 mM to 40 mM. In embodiments, a buffer is a histidine buffer such as histidine/histidine-HCl and is at a concentration from about 15 mM to 25 mM. In embodiments, a buffer is a histidine buffer such as histidine/histidine-HCl and is at a concentration from about 18 mM to 22 mM.

In embodiments, a buffer is at a concentration from about, at least about, or at most about: 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, or 40 mM. In embodiments, a buffer is a histidine buffer such as histidine/histidine-HCl and is at a concentration from about 20 mM, 21 mM, 22 mM, 23 mM, 24 mM, 25 mM, 26 mM, 27 mM, 28 mM, 29 mM, or 30 mM. In embodiments, a buffer is a histidine buffer at a concentration from about, up to about, or at least about 20 mM. In embodiments, a buffer is histidine/histidine-HCl at a concentration from about, up to about, or at least about 20 mM. In embodiments, a buffer is histidine/histidine-HCl at a concentration of 20 mM.

### Potential of Hydrogen

A composition provided herein comprises a potential of hydrogen (pH). In embodiments, a pH is acidic. In embodiments, a pH is neutral. In embodiments, a pH is basic. In embodiments, a composition has a pH from about 6 to about 7. In embodiments, a composition has a pH of about 7. In embodiments, a composition has a pH from about 6.5 to about 7.

In embodiments, a composition has a pH from about 5 to about 7. In embodiments, a composition has a pH from about 6 to about 7. In embodiments, a composition has a pH from about 6.5 to about 7. In embodiments, a composition has a pH from about 6.8 to about 7. In embodiments, a composition has a pH from about 6.9 to about 7.1 In embodiments, a composition has a pH from about 6 to about 8. In embodiments, a composition has a pH from about 5.6 to about 6. In embodiments, a composition has a pH from about 6 to about 6.1. In embodiments, a composition has a pH from about 7 to about 9.

In embodiments, a composition provided herein has a pH of about: 5, 6, 7, or 8. In embodiments, a composition provided herein has a pH of about 6.

pH can be determined using a pH meter or similar technology in the art.

### Pharmaceutical Compositions Comprising an Anti-ILT7 Binding Protein

The present disclosure is also directed to pharmaceutical compositions comprising an anti-ILT7-binding protein as described herein. In embodiments, the present disclosure provides for the use of a pharmaceutical composition comprising an anti-ILT7 binding protein (e.g., mAb) used in the methods described herein in the manufacture of a medicament for treating a subject.

In embodiments, a pharmaceutical composition of the disclosure comprises an ILT7-binding protein disclosed herein and one or more pharmaceutically acceptable carriers, diluents, or excipients. In this regard, “pharmaceutically acceptable carriers, diluents, or excipients” include but are not limited to any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier that may or may not have been approved by the United States Food and Drug Administration as being acceptable for use in humans or domestic animals. In embodiments, carriers are known to those skilled in the art and include stabilizers, diluents, and buffers. In embodiments, stabilizers may include but are not limited to polyanions, salts, chelators (e.g., EDTA), metal ions (e.g., Cu²⁺, Zn²⁺), carbohydrates, (e.g., sorbitol, lactose, mannitol, starch, sucrose, dextran, and glucose, and the like), polysorbates (PS; e.g., PS-20, PS-40, PS-60, PS-80), proteins, (e.g., albumin or casein, and the like), and amino acids (e.g., arginine, aspartic acid, glutamic acid, lysine, proline, glycine, histidine, methionine). In embodiments, diluents may include but are not limited to saline, Hanks Balanced Salts, and Ringers solution, and the like.

In embodiments, a composition is formulated into a pharmaceutical composition. In embodiments, a formulation comprises: from 100-200 mg/mL anti-ILT7 mAb, from 10-30 mM histidine/histidine-HCl, from 150-200 mM sucrose, from 10-20 mM L-methionine, from 0.01-0.05% PS-80, and pH from 5.5-6.5. In embodiments, a formulation comprises: 150 mg/mL anti-ILT7 mAb, 20 mM histidine/histidine-HCl, 180 mM sucrose, 15 mM L-methionine, 0.02% PS-80, and pH 6. In embodiments, a component of a formulation can range in amount by about 25%. Exemplary amounts are described herein.

In embodiments, an anti-ILT7 mAb of the disclosure and a composition comprising the same may be used in vitro, ex vivo, or incorporated into a pharmaceutical composition. In embodiments, an anti-ILT7 mAb of the disclosure and a composition comprising the same may be used in vitro, ex vivo, or incorporated into a pharmaceutical composition and administered to a subject (e.g., to treat, ameliorate, or prevent disease, or combinations thereof). In embodiments, the administration of a pharmaceutical composition is not dependent on the formulation (e.g., dosage). In embodiments, the administration of a pharmaceutical composition may be dependent on the formulation (e.g., dosage). In embodiments, the administration of a pharmaceutical composition of the disclosure may be dependent on formulation (e.g., dosage) and on a therapeutically effective amount for a subject (e.g., to treat, ameliorate, or prevent disease, or combinations thereof). In embodiments, a pharmaceutical composition of the disclosure is formulated into preparations in solid, semi-solid, liquid or gaseous forms, including, but not limited to, solutions, injections, liquid compositions, microspheres, and aerosols. In embodiments, a pharmaceutical composition comprising an anti-ILT7 binding protein used in the methods described herein may be in the form of a solid or liquid. In embodiments, a carrier is liquid, (e.g., an injectable liquid). In embodiments, a carrier (e.g., an injectable liquid) is used for administration (e.g., intravenous or subcutaneous).

### Route of Administration

In embodiments, any of the compositions comprising an anti-ILT7 binding protein of the disclosure can be administered in any form. In embodiments, an anti-ILT7 binding protein is administered intravenously, subcutaneously, orally, intramuscularly, intrathecally, sublingually, rectally, vaginally, cutaneously, systemically, topically, transdermally, or by way of inhalation. In embodiments, an anti-ILT7-binding protein is administered intravenously. In embodiments, an anti-ILT7-binding protein is administered subcutaneously. In embodiments, an anti-ILT7-binding protein is administered subcutaneously via autoinjector.

In embodiments, disclosed compositions are formulated and loaded into a self-administrable delivery device. Exemplary devices are described herein and include exemplary devices such as autoinjectors. An autoinjector can allow for an intramuscular or subcutaneous route of administration. Any site of a subject can be targeted for use with an autoinjector. In embodiments, an injector target site is selected from the group consisting of: thigh, buttock, abdomen, arm, loin, and combinations thereof. In embodiments, an injector is contacted with a thigh of a subject to effectuate delivery of a composition of the disclosure. In embodiments, an injector is contacted with the abdomen of a subject to effectuate delivery of a composition of the disclosure. In embodiments, an injector is contacted with the buttock of a subject to effectuate delivery of a composition of the disclosure.

Alternatively, a disclosed composition can be administered orally as a liquid, capsule, or tablet (e.g., chewable tablet). Because the oral route is the most convenient and usually the safest and least expensive, it is the one most often used. However, it has limitations because of the way a drug typically moves through the digestive tract. For drugs administered orally, absorption may begin in the mouth and stomach. However, most drugs are usually absorbed from the small intestine. The drug passes through the intestinal wall and travels to the liver before being transported via the bloodstream to its target site. The intestinal wall and liver chemically alter (metabolize) many drugs, decreasing the amount of drug reaching the bloodstream. Consequently, these drugs are often given in smaller doses when injected intravenously to produce the same effect.

For a subcutaneous route, a needle is inserted into fatty tissue just beneath the skin. After a drug is injected, it then moves into small blood vessels (capillaries) and is carried away by the bloodstream. Alternatively, a drug reaches the bloodstream through the lymphatic vessels. The intramuscular route is preferred to the subcutaneous route when larger volumes of a drug product are needed. Because the muscles lie below the skin and fatty tissues, a longer needle is used. Drugs are usually injected into the muscle of the upper arm, thigh, or buttock. How quickly the drug is absorbed into the bloodstream depends, in part, on the blood supply to the muscle: The sparser the blood supply, the longer it takes for the drug to be absorbed. For the intravenous route, a needle is inserted directly into a vein. A solution containing the drug may be given in a single dose or by continuous infusion. For infusion, the solution is moved by gravity (from a collapsible plastic bag) or, more commonly, by an infusion pump through thin flexible tubing to a tube (catheter) inserted in a vein, usually in the forearm.

### Timing of Administration

A pharmaceutical composition of the disclosure can be administered at any time. In embodiments, a composition is administered before, during, or after symptoms of a described disease or composition are detected. In embodiments, a composition is administered by way of a delivery device (e.g., an autoinjector). In embodiments, a composition of the disclosure is administered every 4 weeks, 5 weeks, 6 weeks, 8 weeks, 10 weeks, 20 weeks, 25 weeks, 30 weeks, or 50 weeks. In embodiments, a composition of the disclosure is administered every month, 2 months, 3 months, 4 months, 5 months, 6 months, 7 months, 8 months, 9 months, 10 months, 11 months, 1 year, 2 years, 3 years, 4 years or 5 years.

In embodiments, a pharmaceutical composition provided herein is administered via infusion or bolus injection. An infusion can take place over a period of time. For example, an infusion can be an administration of a pharmaceutical over a period of about 5 minutes to about 10 hours. An infusion can take place over a period of about 5 min, 10 min, 20 min, 30 min, 40 min, 50 min, 1 hour, 1.5 hours, 2 hours, 2.5 hours, 3 hours, 3.5 hours, 4 hours, 4.5 hours, 5 hours, 6 hours, 7 hours, 8 hours, 9 hours, or up to about 10 hours. In embodiments, intravenous administration is used to deliver a precise dose quickly and in a well-controlled manner throughout the body. It is also used for irritating solutions, which would cause pain and damage tissues if given by subcutaneous or intramuscular injection. When given intravenously, a drug is delivered immediately to the bloodstream and tends to take effect more quickly than when given by any other route. Consequently, health care practitioners closely monitor people who receive an intravenous injection for signs that the drug is working or is causing undesired side effects. Also, the effect of a drug given by this route tends to last for a shorter time. Therefore, some drugs must be given by continuous infusion to keep their effect constant. In embodiments, infusion reactions can occur and include headache, nausea, somnolence, dyspnea, fever, myalgia, rash, or other symptoms. Potential risks associated with administration of an anti-ILT7 binding protein are infection, redness, swelling, pain, and induration at the administration site. Prior to each IV infusion subjects may receive prophylaxis with IV methylprednisolone, oral diphenhydramine, and oral acetaminophen, or equivalent(s) to reduce the risk or severity of potential reactions.

In embodiments, a pharmaceutical is administered intrathecally. For the intrathecal route, a needle is inserted between two vertebrae in the lower spine and into the space around the spinal cord. The drug is then injected into the spinal canal. A small amount of local anesthetic is often used to numb the injection site. This route is used when a drug is needed to produce rapid or local effects on the brain, spinal cord, or the layers of tissue covering them (meninges)—for example, to treat infections of these structures. Drugs administered by inhalation through the mouth can be atomized into smaller droplets than those administered by the nasal route, so that the drugs can pass through the windpipe (trachea) and into the lungs. How deeply into the lungs they go depends on the size of the droplets. Smaller droplets go deeper, which increases the amount of drug absorbed. Inside the lungs, they are absorbed into the bloodstream. Drugs applied to the skin are usually used for their local effects and thus are most commonly used to treat superficial skin disorders, such as psoriasis, eczema, skin infections (viral, bacterial, and fungal), itching, and dry skin. The drug is mixed with inactive substances. Depending on the consistency of the inactive substances, the formulation may be an ointment, cream, lotion, solution, powder, or gel.

In embodiments, a treatment regime comprising a pharmaceutical composition may be dosed according to a body weight of a subject. In subjects who are determined obese (BMI >35) a practical weight may need to be utilized. In embodiments, body surface area may be utilized to calculate a dosage.

### Formulations

In embodiments, a pharmaceutical composition of the disclosure is in the form of a liquid, for example, a solution, emulsion, or suspension. In embodiments, in a pharmaceutical composition intended to be administered by injection, one or more of a surfactant, preservative, wetting agent, dispersing agent, suspending agent, buffer, stabilizer and isotonic agent may be included. In embodiments, a pharmaceutical composition of the disclosure is administered to a subject in need thereof intravenously. In embodiments, a pharmaceutical composition of the disclosure is administered to a subject in need thereof by subcutaneous injection. In embodiments, a pharmaceutical composition of the disclosure is administered to a subject in need thereof by subcutaneous injection via autoinjector.

In embodiments, liquid pharmaceutical compositions comprising an ILT7-binding protein used in the methods described herein, whether they be solutions, suspensions or other like form, may include one or more of the following components: sterile diluents such as water for injection, saline solution, e.g., physiological saline, Ringer's solution, isotonic sodium chloride, fixed oils such as synthetic monoglycerides or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. In embodiments, the preparation can be enclosed in one or more ampoules, disposable syringes or vials (e.g., glass or plastic). In embodiments, the preparation is enclosed in one or more vials (e.g., glass or plastic). In embodiments, the preparation is enclosed in one or more disposable syringes, e.g., a prefilled syringe (PFS). In embodiments, the preparation is enclosed in an autoinjector. In embodiments, the preparation is enclosed in a pen. In embodiments, the preparation is enclosed in one or more vials (e.g., glass or plastic) prior to administration and is then transferred to an autoinjector for administration to a subject. In embodiments, the preparation is enclosed in one or more disposable syringes (e.g., a PFS) prior to administration and is then transferred to an autoinjector for administration to a subject. In embodiments, an injectable pharmaceutical composition is sterile and compliant with GMP practice.

In embodiments, an anti-ILT7 binding protein of the disclosure and compositions comprising the same may be used as a pharmaceutical composition and can be combined with a pharmaceutically acceptable carrier. In embodiments, a composition may comprise an anti-ILT7-binding protein, carriers, various diluents, fillers, salts, buffers, stabilizers, solubilizers, and other materials well known in the art. In embodiments, characteristics of a carrier may depend on a route of administration. In embodiments, a pharmaceutical composition comprises a histidine buffer (e.g., histidine/histidine-HCl). In embodiments, pharmaceutical compositions of the disclosure comprise a pharmaceutically acceptable, non-toxic, sterile carrier. In embodiments, a pharmaceutical composition of the disclosure comprises an anti-ILT7 binding protein disclosed herein and one or more pharmaceutically acceptable carriers, diluents, or excipients. In this regard, “pharmaceutically acceptable carriers, diluents, or excipients” include but are not limited to any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier that may or may not have been approved by the United States Food and Drug Administration as being acceptable for use in humans or domestic animals. For example, appropriate carriers are known to those skilled in the art and include stabilizers, diluents, and buffers. Exemplary stabilizers include carbohydrates (sorbitol, lactose, mannitol, starch, sucrose, dextran, and glucose, and the like) and proteins (e.g., albumin or casein). Exemplary diluents include saline, Hanks Balanced Salts, and Ringer's solution, and the like. Exemplary buffers include an alkali metal phosphate, an alkali metal carbonate, or an alkaline earth metal carbonate, and the like.

In embodiments, a pharmaceutical composition of the disclosure may comprise one or more auxiliary substance, such one or more lipids, phospholipids, carbohydrates, and lipopolysaccharides. In embodiments, pharmaceutical compositions of the disclosure may comprise one or more additional active substances. In embodiments, a pharmaceutical composition of the disclosure comprises one or more additional therapeutically active substances. In embodiments, a therapeutically effective dose of the pharmaceutical compositions of the disclosure is administered to a subject in need thereof in combination with one or more additional therapeutically active substances. As used herein, a “combination” refers to a combination comprising an anti-ILT7-binding protein provided herein and one or more additional therapeutically active substances, each of which may be administered serially (sequentially), concurrently or simultaneously. In embodiments, pharmaceutical compositions of the disclosure may be administered at several intervals in order to sustain therapeutic levels. In embodiments, pharmaceutical compositions of the disclosure may be used in conjunction with other bactericidal or bacteriostatic methods.

In embodiments, pharmaceutical compositions suitable for administration may be sterile. Prevention of the action of microorganisms can be achieved by various antibacterial and antifungal agents. Suitable formulations for use in the therapeutic methods disclosed herein are described in Remington's Pharmaceutical Sciences (Mack Publishing Co.) 16th ed. (1980).

Therapeutically effective doses of the compositions of the disclosure, for treatment of ILT7-expressing cell-mediated diseases such as certain types of autoimmune diseases vary depending upon many different factors, including means of administration, target site, physiological state of the subject, whether a subject is human or a non-human animal, other medications administered, and whether treatment is prophylactic or therapeutic. In embodiments, a subject is a human. In embodiments, a subject is a non-human animal (e.g., transgenic mammals). In embodiments, treatment dosages can be titrated to optimize safety and efficacy.

In embodiments, pharmaceutical compositions for use in the disclosed methods may be manufactured in a conventional manner. In embodiments, the pharmaceutical composition is provided in lyophilized form. In embodiments, a pharmaceutical composition in lyophilized form is dissolved in a suitable aqueous carrier, for example sterile water for injection or sterile buffered physiological saline prior to administration to a subject in need thereof. In embodiments, a pharmaceutical composition is prepared for infusion. In embodiments, a pharmaceutical composition is prepared for infusion and an albumin (e.g., human serum albumin [HSA]) or the subject's own heparinized blood is incorporated into the saline at the time of formulation. The presence of an excess of such physiologically inert protein prevents loss of antibody by adsorption onto the walls of the container and tubing used with the infusion solution. In embodiments, a pharmaceutical composition is prepared for infusion and an albumin is used. In embodiments, a pharmaceutical composition is prepared for infusion and an albumin is used at a concentration from about 0.5 to about 4.5% by weight of the saline solution. Other formulations can comprise liquid or lyophilized formulation.

In embodiments, a lyophilizate of the disclosure comprises an anti-ILT7 binding protein. In embodiments, a lyophilizate comprising an anti-ILT7 binding protein may be reconstituted to give aqueous compositions with an anti-ILT7 binding protein concentration of at least about 150 mg/mL. In embodiments, a lyophilizate may comprise, in addition to an anti-ILT7 binding protein, further components such as one or more of the following: (i) a sugar (e.g., sucrose); (ii) a buffering agent (e.g., histidine/histidine-HCl); (iii) PS-80; and (iv) a methionine (e.g., L-methionine). Exemplary reconstituents for lyophilized binding proteins (e.g., antibodies) include sterile water, buffer, and/or a preservative. In embodiments, a lyophilizate comprises a buffering agent and a reconstituent comprises a buffering agent, which may be the same as or different from the lyophilizate's buffering agent. In embodiments, a lyophilizate comprises a buffering agent and a reconstituent does not comprise a buffering agent (e.g., sterile water for injection [WFI], physiological saline). In embodiments, a reconstituate comprises pharmacological agents, such as chemotherapeutic compounds, which may facilitate co-delivery together with the binding protein (e.g., antibody). In embodiments, a reconstituate comprises buffers, salts, stabilizing agents, glycerol, alcohols, preservatives, surfactants, etc. A thorough discussion of such pharmaceutical ingredients is available in Gennaro (2000) Remington: The Science and Practice of Pharmacy. 20th edition, ISBN: 0683306472.

In embodiments, the pharmaceutical compositions of the disclosure can be prepared by techniques known to those skilled in the art. General considerations in the formulation and/or manufacture of pharmaceutical compositions may be found, for example, in Remington: The Science and Practice of Pharmacy 21st ed., Lippincott Williams & Wilkins, 2005 (incorporated herein by reference in its entirety). In embodiments, an anti-ILT7 binding protein used in the methods described herein or fragments thereof may be mixed with a carrier to form a solution, suspension, or emulsion. In embodiments, one or more of the additives discussed herein may be added to the carrier or may be added subsequently. In embodiments, a pharmaceutical composition of the disclosure may be an aqueous solution, e.g., emulsion or suspension, or may be a dried preparation. In embodiments, a pharmaceutical composition of the disclosure may be desiccated or lyophilized, for example, by freeze drying or spray drying for storage or formulations purposes. In embodiments, a desiccated or lyophilized pharmaceutical composition may be reconstituted into liquid compositions by addition of a liquid carrier. In embodiments, a desiccated or lyophilized pharmaceutical composition may be administered in dry formulation using methods known to those skilled in the art. In embodiments, an anti-ILT7 binding protein used in the methods described herein is stored as lyophilized powder and subsequently reconstituted into liquid compositions prior to administration to a subject in need thereof. In embodiments, a pharmaceutical composition comprising an anti-ILT7 binding protein is stored as lyophilized powder and subsequently reconstituted into liquid compositions prior to administration to a subject in need thereof.

### Delivery Device

The various compositions of the disclosure can be loaded into a delivery device or be pre-loaded and stored for future use. Drug delivery devices, such as injectors, are used to deliver liquid drugs to a subject in need thereof. Upon activation, a drug delivery device may expel a drug (e.g., an anti-ILT7 antibody) stored within an internal reservoir of a primary container through a needle, cannula, or other delivery member into the subject.

Exemplary delivery devices include but are not limited to pen injectors, manual injectors, autoinjectors and on-body devices. In embodiments, a delivery device delivers e.g., a medicament, a composition, a formulation, and the like, by automatic administration. In embodiments, a delivery device is a pen injector. In embodiments, a delivery device is an autoinjector. In embodiments, a delivery device is an on-body device. In embodiments, a delivery device is a manual injector. In embodiments, a delivery device and a storage device are the same. In embodiments, a delivery device delivers an anti-ILT7 binding protein or a pharmaceutical composition comprising an anti-ILT7 binding protein to a delivery site, e.g., through the skin of a subject in need thereof. In embodiments, a delivery device is wearable.

In embodiments, delivery devices, such as autoinjectors and manual injectors, comprise a needle shield that extends in the forward direction from the housing and a needle. In embodiments, the needle shield protects the needle from exposure before delivery. In embodiments, the needle shield may be arranged to move along the longitudinal axis of the housing further into the housing when the medicament delivery device is pushed towards the delivery site. In embodiments, for autoinjectors, this movement of the needle shield may cause the medicament delivery device to become activated to discharge medicament via the needle.

Provided are autoinjectors pre-loaded with 150 mg/mL of an anti-ILT7 antibody in 20 mM histidine, 15 mM methionine, 180 mM sucrose, 0.02% PS80, at pH 6.0. In embodiments, an autoinjector is a single-use or disposable injector.

In embodiments, provided are devices comprising a subassembly of a medicament delivery device, the subassembly comprising a housing configured to contain a medicament container structure from which medicament (e.g., an anti-ILT7 antibody) is expelled during use of the medicament delivery device, the housing having a proximal end and a distal end, and a drive assembly comprising a plunger rod at a proximal end of the drive assembly, the plunger rod being configured to, upon activation, apply an activating force on the medicament container structure for expelling the medicament from the medicament delivery device, wherein the proximal end of the housing comprises an opening, and the distal end comprises a sealable portion, wherein the drive assembly is insertable into the housing at the proximal end to form the subassembly. In embodiments, the medicament is expelled following contact with a target injection site (e.g., abdomen, thigh, and/or arm). In embodiments, the medicament is expelled following activation by button.

Exemplary delivery devices (e.g., autoinjectors), and methods of utilizing the same are described in International Patent Publication Nos. WO/2023/016889, WO/2023/016893, WO/2023/016933, WO/2023/016888, WO/2022/212227, WO/2022/203830, WO/2022/192100, WO/2022/192308, WO/2022/098593, WO/2022/098592, WO/2021/011716, WO/2021/011717, WO/2020/023220, and WO/2018/034784, which are incorporated by reference herein in their entireties. For example, in embodiments, a delivery device described in any of the aforementioned patent applications can be utilized to deliver a composition of the disclosure.

The devices, assemblies, components, subsystems, methods or drug delivery devices can further comprise or be used with a drug, including but not limited, to those compositions described herein as well as any variations thereof. In embodiments, the composition may be in liquid form, a lyophilized form, or in a reconstituted from lyophilized form.

### Kits Comprising an Anti-ILT7 Binding Protein

Disclosed herein are kits (i.e., an article of manufacture) useful for providing an anti-ILT7 binding protein composition, e.g., anti-ILT7 antibody. Such kits may comprise an anti-ILT7 binding protein (e.g., in liquid or lyophilized form) or a pharmaceutical composition comprising the anti-ILT7 binding protein. Additionally, such kits may comprise an anti-ILT7 binding protein or pharmaceutical composition comprising an anti-ILT7 binding protein enclosed in a storage container, (e.g., a syringe, a prefilled pen, a vial), a delivery device (e.g., an autoinjector, an on-body device, a pen injector) and instructions for use. These kits may contain additional therapeutic agents for treating autoimmune diseases described supra, e.g., for delivery in combination with the enclosed anti-ILT7 binding protein.

In embodiments, a kit comprises a plurality of delivery devices. For example, a kit can comprise from 1, 2, 3, 4, 5, 6, 7, 8, 9, or up to 10 delivery devices (e.g., autoinjectors). In embodiments, a kit comprises disposable autoinjectors. In embodiments, a kit also comprises instructions for use thereof. In embodiments, a kit also comprises means of disposing a used autoinjector (e.g., sharps container).

In embodiments, provided are kits comprising: a) a pre-filled autoinjector comprising a pharmaceutical composition comprising an anti-ILT7 binding protein; and b) instructions for use thereof. In embodiments, a pre-filled autoinjector comprises daxdilimab.

In embodiments, provided are kits comprising: a) a pharmaceutical composition comprising an anti-ILT7 binding protein composition for administration to a subject in need thereof; b) instructions describing how to administer said pharmaceutical composition to the subject in need thereof; and c) a delivery device to deliver the pharmaceutical composition to the subject in need thereof.

In embodiments, provided are kits comprising: a) a pharmaceutical composition comprising an anti-ILT7 binding protein composition for administration to a subject in need thereof, wherein the subject has an autoimmune disease; b) instructions describing how to administer said pharmaceutical composition to the subject in need thereof; and c) a delivery device to deliver the pharmaceutical composition to the subject in need thereof. Exemplary autoimmune diseases include but are not limited to focal segmental glomerulosclerosis (FSGS), alopecia areata, myositis, diabetes, Hashimoto's disease, autoimmune adrenal insufficiency, pure red cell anemia, multiple sclerosis, IgG4RD, rheumatoid carditis, systemic lupus erythematosus, psoriasis, rheumatoid arthritis, chronic inflammation, Sjögren's syndrome, polymyositis, dermatomyositis, inclusion body myositis, juvenile myositis, and scleroderma.

Provided are containers comprising an anti-ILT7 binding protein (e.g., daxdilimab). In embodiments, provided herein are containers that comprise compositions in unit dose form.

In embodiments, a container is a vial. In embodiments, the vial is glass. In embodiments, the vial is plastic. In embodiments, a vial may comprise a lyophilized dosage form of a composition provided herein.

In embodiments, a vial may comprise a liquid dosage form of a composition provided herein. In embodiments, a container is a vial and contains about 0.1 mL, 0.2 mL, 0.3 mL, 0.4 mL, 0.5 mL, 0.6 mL, 0.7 mL, 0.8 mL, 0.9 mL, 1.0 mL, 1.25 mL, 1.5 mL, 1.75 mL, 2.0 mL, 2.25 mL, 2.5 mL, 2.75 mL, 3.0 mL, 3.25 mL, 3.5 mL, 3.75 mL, 4.0 mL, 4.25 mL, 4.5 mL, 4.75 mL, 5.0 mL, 5.25 mL, 5.5 mL, 5.75 mL, 6.0 mL, 6.25 mL, 6.5 mL, 6.75 mL, 7.0 mL, 7.25 mL, 7.5 mL, 7.75 mL, 8.0 mL, 8.25 mL, 8.5 mL, 8.75 mL, 9.0 mL, 9.25 mL, 9.5 mL, 9.75 mL, or about 10.0 mL of a liquid dosage form. In embodiments, a container is a vial and contains about 0.1 mL to about 0.5 mL, about 0.1 mL to about 1.0 mL, about 0.5 mL to about 1.5 mL, about 0.7 mL to about 2.0 mL, about 1.0 mL to about 2.5 mL, about 1.25 mL to about 2.0 mL, about 1.25 mL to about 2.75 mL, about 2.0 mL to about 4.0 mL, about 1.5 mL to about 3.0 mL, about 3.0 mL to about 5.0 mL, about 4.0 mL to about 6.0 mL, about 5.0 mL to about 7.0 mL, about 6.0 mL to about 8.0 mL, about 7.0 mL to about 9.0 mL, about 8.0 mL to about 10.0 mL, or about 9.0 mL to about 10.0 mL of a liquid dosage form. In embodiments, a container is a vial a contains about 1.0 mL of a liquid dosage. In embodiments, a container is a vial a contains about 1.5 mL of a liquid dosage. In embodiments, a container is a vial a contains about 2.0 mL of a liquid dosage.

In embodiments, a container is a syringe (e.g., a PFS). In embodiments, a syringe is a PFS. In embodiments, a vial may comprise a liquid dosage form of a composition provided herein. In embodiments, a container is a PFS and contains about 0.1 mL, 0.2 mL, 0.3 mL, 0.4 mL, 0.5 mL, 0.6 mL, 0.7 mL, 0.8 mL, 0.9 mL, 1.0 mL, 1.25 mL, 1.5 mL, 1.75 mL, 2.0 mL, 2.25 mL, 2.5 mL, 2.75 mL, 3.0 mL, 3.25 mL, 3.5 mL, 3.75 mL, 4.0 mL, 4.25 mL, 4.5 mL, 4.75 mL, 5.0 mL, 5.25 mL, 5.5 mL, 5.75 mL, 6.0 mL, 6.25 mL, 6.5 mL, 6.75 mL, 7.0 mL, 7.25 mL, 7.5 mL, 7.75 mL, 8.0 mL, 8.25 mL, 8.5 mL, 8.75 mL, 9.0 mL, 9.25 mL, 9.5 mL, 9.75 mL, or about 10.0 mL of a liquid dosage form. In embodiments, a container is a PFS and contains about 0.1 mL to about 0.5 mL, about 0.1 mL to about 1.0 mL, about 0.5 mL to about 1.5 mL, about 0.7 mL to about 2.0 mL, about 1.0 mL to about 2.5 mL, about 1.25 mL to about 2.0 mL, about 1.25 mL to about 2.75 mL, about 2.0 mL to about 4.0 mL, about 1.5 mL to about 3.0 mL, about 3.0 mL to about 5.0 mL, about 4.0 mL to about 6.0 mL, about 5.0 mL to about 7.0 mL, about 6.0 mL to about 8.0 mL, about 7.0 mL to about 9.0 mL, about 8.0 mL to about 10.0 mL, or about 9.0 mL to about 10.0 mL of a liquid dosage form. In embodiments, a container is a PFS a contains about 1.0 mL of a liquid dosage. In embodiments, a container is a PFS a contains about 1.5 mL of a liquid dosage. In embodiments, a container is a PFS a contains about 2.0 mL of a liquid dosage.

In embodiments, a formulation is stable as a liquid formulation, e.g., in liquid form. In embodiments, a formulation in liquid form facilitates ease of administration as compared to an otherwise comparable formulation not in liquid form. In embodiments, a formulation in liquid form comprises fewer manufacturing steps as compared to an otherwise comparable formulation not in liquid form.

In embodiments, provided are kits comprising a delivery device. In embodiments, a delivery device is e.g., an injection device, an inhalation device, or a medical sprayer. In embodiments, a delivery device is an autoinjector.

In embodiments, a kit comprises a delivery device, wherein the delivery device is an assembled delivery device prior to addition to the kit. In embodiments, a kit comprises a delivery device, wherein the delivery device is a partially assembled delivery device prior to addition to the kit. In embodiments, a kit comprises a delivery device, wherein the delivery device is a completely assembled delivery device prior to addition to the kit. In embodiments, a kit comprises a delivery device, wherein the delivery device is an assembled delivery device prior to administration. In embodiments, a kit comprises a delivery device, wherein the delivery device is a completely assembled delivery device prior to administration.

In embodiments, a kit comprises a delivery device, wherein the delivery device is assembled as part of the kit instructions. In embodiments, an anti-ILT7 binding protein or a pharmaceutical composition comprising an anti-ILT7 binding protein enclosed in a storage container is combined with a delivery device prior to administration. In embodiments, a kit comprises instructions, wherein the instructions describe how to combine a delivery device and an anti-ILT7 binding protein or a pharmaceutical composition comprising an anti-ILT7 binding protein enclosed in a storage container. In embodiments, a kit comprises instructions, wherein the instructions describe how to combine a delivery device and an anti-ILT7 binding protein or a pharmaceutical composition comprising an anti-ILT7 binding protein enclosed in a storage container, thereby producing a completely assembled delivery device for delivery to a delivery site (e.g., through skin of a subject in need thereof).

In embodiments, provided are kits comprising a) a pharmaceutical composition comprising an anti-ILT7 binding protein composition for administration to a subject in need thereof, wherein the subject has an autoimmune disease; b) instructions describing how to administer said pharmaceutical composition to a subject in need thereof; and, c) a delivery device for delivery of the pharmaceutical composition to the subject in need thereof.

In embodiments, provided are kits comprising a) a pharmaceutical composition comprising an anti-ILT7 binding protein composition for administration to a subject in need thereof, wherein the subject has an autoimmune disease; b) instructions describing how to administer said pharmaceutical composition to a subject in need thereof; and, c) an autoinjector for delivery of the pharmaceutical composition to the subject in need thereof.

In embodiments, a formulation disclosed herein is utilized for subcutaneous administration. In embodiments, a volume of a formulation of the disclosure administered subcutaneously is from about 0.5 mL to about 2.5 mL. In embodiments, a volume of a formulation of the disclosure administered subcutaneously is about 0.5 mL, 0.55 mL, 0.6 mL, 0.65 mL, 0.7 mL, 0.75 mL, 0.8 mL, 0.85 mL, 0.9 mL, 0.95 mL, 1 mL, 1.05 mL, 1.1 mL, 1.15 mL, 1.2 mL, 1.25 mL, 1.3 mL, 1.35 mL, 1.4 mL, 1.45 mL, 1.5 mL, 1.55 mL, 1.6 mL, 1.65 mL, 1.7 mL, 1.75 mL, 1.8 mL, 1.85 mL, 1.9 mL, 1.95 mL, 2 mL, 2.05 mL, 2.1 mL, 2.15 mL, 2.2 mL, 2.25 mL, 2.3 mL, 2.35 mL, 2.4 mL, 2.45 mL, or up to about 2.5 mL. In embodiments, a volume of a formulation of the disclosure administered subcutaneously is about 0.5 to about 1.0 mL, about 0.5 mL to about 1.5 mL, about 1.0 mL to about 2.0 mL, about 0.75 mL to about 2.25 mL, about 1.0 mL to about 2.5 mL, about 1.5 mL to about 2.5 mL, or about 1.0 mL to about 2.25 mL.

In embodiments, a volume of a formulation of the disclosure is administered subcutaneously is about 1.0 mL. In embodiments, a volume of a formulation of the disclosure is administered subcutaneously is about 1.5 mL. In embodiments, a volume of a formulation of the disclosure is administered subcutaneously is about 2.0 mL. In embodiments, a volume of a formulation of the disclosure is administered subcutaneously is about 1.0 mL, wherein the formulation was stored in a syringe (e.g., pre-filled syringe; PFS) prior to administration. In embodiments, a volume of a formulation of the disclosure is administered subcutaneously is about 1.5 mL wherein the formulation was stored in a syringe (e.g., pre-filled syringe; PFS) prior to administration. In embodiments, a volume of a formulation of the disclosure is administered subcutaneously is about 2.0 mL wherein the formulation was stored in a syringe (e.g., pre-filled syringe; PFS) prior to administration. In embodiments, a volume of a formulation of the disclosure is administered subcutaneously is about 1.0 mL, wherein the formulation was stored in two or more syringes (e.g., pre-filled syringe; PFS) prior to administration. In embodiments, a volume of a formulation of the disclosure is administered subcutaneously is about 1.5 mL wherein the formulation was stored in in two or more syringes (e.g., pre-filled syringe; PFS) prior to administration. In embodiments, a volume of a formulation of the disclosure is administered subcutaneously is about 2.0 mL wherein the formulation was stored in two or more syringes (e.g., pre-filled syringe; PFS) prior to administration.

In embodiments, a volume of a formulation of the disclosure is administered subcutaneously at about 1.0 mL, wherein the formulation was stored in two or more syringes (e.g., pre-filled syringe; PFS) prior to administration and the formulation is administered by a delivery device. In embodiments, a volume of a formulation of the disclosure is administered subcutaneously at about 1.5 mL wherein the formulation was stored in in two or more syringes (e.g., pre-filled syringe; PFS) prior to administration and the formulation is administered by a delivery device. In embodiments, a volume of a formulation of the disclosure is administered subcutaneously is about 2.0 mL wherein the formulation was stored in two or more syringes (e.g., pre-filled syringe; PFS) prior to administration and the formulation is administered by a delivery device.

In embodiments, a volume of a formulation of the disclosure is administered subcutaneously is about 1.0 mL, wherein the formulation was stored in two or more syringes (e.g., pre-filled syringe; PFS) prior to administration and the formulation is administered by a by an autoinjector. In embodiments, a volume of a formulation of the disclosure is administered subcutaneously is about 1.5 mL wherein the formulation was stored in in two or more syringes (e.g., pre-filled syringe; PFS) prior to administration and the formulation is administered by an autoinjector. In embodiments, a volume of a formulation of the disclosure is administered subcutaneously is about 2.0 mL wherein the formulation was stored in two or more syringes (e.g., pre-filled syringe; PFS) prior to administration and the formulation is administered by an autoinjector.

In embodiments, a formulation disclosed herein comprises high protein concentration of a binding protein (e.g., an anti-ILT7 binding protein) yet retains viscosity levels suitable for subcutaneous administration. In embodiments, high protein concentration facilitate higher doses that can be delivered in a single injection. In embodiments, a range of viscosity levels in the formulations disclosed herein is about 1 cP to about 50 cP. In embodiments, a formulation of the disclosure comprises a viscosity of about 0 cP, 2 cP, 4 cP, 6 cP, 8 cP, 10 cP, 12 cP, 14 cP, 16 cP, 18 cP, 20 cP, 22 cP, 24 cP, 26 cP, 28 cP, 30 cP, 32 cP, 34 cP, 36 cP, 38 cP, 40 cP, 42 cP, 44 cP, 46 cP, 48 cP, or up to about 50 cP. In embodiments, a range of viscosity levels in the formulations of the disclosure range from about 1 cP to about 20 cP, 5 cP to about 30 cP, 10 cP to about 35 cP, 25 cP to about 35 cP, or about 30 cP to about 50 cP.

In embodiments, a single injection of any of the disclosed formulations are administered to a subject in need thereof. In embodiments, multiple injections of any of the disclosed formulations are administered to a subject in need thereof. In embodiments, multiple injections of any of the disclosed formulations may be performed as part of a method of the disclosure. In embodiments, a single injection of any of the disclosed compositions may be performed as part of a method of the disclosure. In embodiments, a subcutaneous administration allows for fewer or single injections of a disclosed composition.

In embodiments, an anti-ILT7 binding protein of the disclosure is administered on a schedule that provides optimal results. In embodiments, an anti-ILT7 binding protein is administered to a subject in need thereof about once a week, about twice a week, about every two weeks, about once a month, about every four weeks, about every two months, about every 3 months, about every 12 weeks, about every fifteen weeks, about every sixteen weeks, about every four months, about every five months, about every six months, or semiannually. Any number of administrations may be provided to a subject in need thereof.

In embodiments, an anti-ILT7 binding protein disclosed herein is administered at a dose of at least about or at most about: 50 mg, 60 mg, 70 mg, 75 mg, 80 mg, 90 mg, 100 mg, 110 mg, 120 mg, 125 mg, 130 mg, 140 mg, 150 mg, 160 mg, 170 mg, 175 mg, 180 mg, 190 mg, 200 mg, 210 mg, 220 mg, 225 mg, 230 mg, 240 mg, 250 mg, 260 mg, 270 mg, 275 mg, 280 mg, 290 mg, 300 mg, 325 mg, 350 mg, 375 mg, 400 mg, 425 mg, 450 mg, 475 mg, 500 mg, 525 mg, 550 mg, 575 mg, or about 600 mg. In embodiments, an anti-ILT7 binding protein is administered on about every week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks. 16 weeks, 20 weeks, or about once a month, about every two months, about every three months, about every four months, about every six months. In embodiments, an anti-ILT7 binding protein is administered at a dose of about 120 mg to about 200 mg every 4 weeks, about 50 mg to about 300 mg every 4 weeks, about 300 mg to about 600 mg every 4 weeks, about 450 mg to about 600 mg every 4 weeks, about 150 mg to about 300 mg every 12 weeks (i.e., every three months), about 200 mg to about 500 mg every 12 weeks (i.e., every three months), or about 300 mg to about 600 mg every 12 weeks (i.e., every three months). In embodiments, an anti-ILT7 binding protein is administered at a dose of about 150 mg every 4 weeks. In embodiments, an anti-ILT7 binding protein is administered at a dose of about 300 mg every 4 weeks. In embodiments, an anti-ILT7 binding protein is administered at a dose of about 450 mg every 4 weeks.

In embodiments, an anti-ILT7 binding protein disclosed herein may be administered at a dose of about 150 mg to about 450 mg subcutaneously in a volume between 1 mL and 2 mL in a single injection. In embodiments, an anti-ILT7 binding protein is administered at a dose of about 300 mg in a single injection of 1 mL. In embodiments, an anti-ILT7 binding protein is administered at a dose of about 300 mg in a single injection of 1.5 mL. In embodiments, an anti-ILT7 binding protein is administered at a dose of about 300 mg in a single injection of 2 mL.

In embodiments, a composition disclosed herein may be administered at a dose of about 150 mg to about 450 mg subcutaneously in a volume between 1 mL and 2 mL in a single injection. In embodiments, a composition is administered at a dose of about 300 mg in a single injection of 1 mL. In embodiments, a composition is administered at a dose of about 300 mg in a single injection of 1.5 mL. In embodiments, a composition is administered at a dose of about 300 mg in a single injection of 2 mL.

In embodiments, a formulation disclosed herein may be administered at a dose of about 150 mg to about 450 mg subcutaneously in a volume between 1 mL and 2 mL in a single injection. In embodiments, a formulation is administered at a dose of about 300 mg in a single injection of 1 mL. In embodiments, a formulation is administered at a dose of about 300 mg in a single injection of 1.5 mL. In embodiments, a formulation is administered at a dose of about 300 mg in a single injection of 2 mL.

### Methods

### Formulation Uses

In embodiments, a formulation comprising an anti-ILT7 binding protein provided herein is effective at resolving one or more of: light-induced tryptophan oxidation, reduced potency, reduced shelf-life, need for nitrogen overlay, and combinations thereof. In embodiments, a formulation does not utilize a nitrogen overlay or lacks a nitrogen overlay in a method of making.

In embodiments, formulations and methods disclosed herein reduce or eliminate the need for engineering controls that include but are not limited to lighting modulation and the need for a nitrogen overlay during manufacturing. In embodiments, a method does not comprise lighting modulation. In embodiments, a method does not comprise a nitrogen overlay. In embodiments, a formulation is generated without use of a nitrogen overlay.

In embodiments, a 15 mM concentration of L-methionine is effective in reducing aggregation, reducing oxidation (e.g., light-induced tryptophan and/or methionine oxidation), or both reducing aggregation and reducing oxidation of a composition provided herein. In embodiments, reduced oxidation can refer to reduced oxidation of L-methionine. In embodiments, reduced oxidation can refer to any one of reduced CDR modification, reduced Fc deamidation (e.g., N330, N389, and/or N394 of SEQ ID NO: 10; or N55 and/or N99 of SEQ ID NO: 1 or SEQ ID NO: 10), reduced Fc oxidation (e.g., M257 and/or M433 of SEQ ID NO: 10), and any combination thereof. In embodiments, reduced oxidation can refer to W102 and/or W104 of SEQ ID NO: 1 or SEQ ID NO: 10. In embodiments, L-methionine is effective at reducing light-induced tryptophan oxidation and/or a rate thereof in a CDR of an anti-ILT7 binding protein. In embodiments, L-methionine is effective in reducing light-induced tryptophan oxidation in a CDR (e.g., a CDR of SEQ ID NO: 1) of an anti-ILT7 binding protein.

The reduction of light-induced tryptophan oxidation can range from stabilization to elimination of oxidation in affected, or presumed to be affected, residues in compositions of the disclosure. The level of oxidation of compositions can be determined at any time. In embodiments, oxidation is determined at the initiation, during an intermediate step, or at the conclusion of manufacturing of disclosed compositions. In embodiments, oxidation can be determined at the conclusion of manufacturing. In embodiments, oxidation can be determined relative to a level before and after addition of L-methionine.

In embodiments, utilization of provided methods is effective at stabilizing a level of oxidation in compositions of the disclosure. The various provided methods can be useful at maintaining oxidation levels of provided compositions (e.g., compositions comprising sequences of Table 1 or compositions with at least about 85% identity to those of Table 1) throughout a manufacturing process and/or throughout transport and subject dosing. Maintenance of oxidation can be useful for preserving potency of provided compositions. For example, compositions of the disclosure can have a stable level of oxidation, of one or more residues, following light exposure. For example, a composition provided herein that is exposed to light at 1600-1800 lux for 3 days has no more than about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12%, 14%, 16%, 18%, 20%, 22%, 24%, 26%, 28%, or 30% oxidized species. For example, a composition provided herein that is exposed to light at 1600-1800 lux for 4 days has no more than about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12%, 14%, 16%, 18%, 20%, 22%, 24%, 26%, 28%, or 30% oxidized species. For example, a composition provided herein that is exposed to light at 1600-1800 lux for 5 days has no more than about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12%, 14%, 16%, 18%, 20%, 22%, 24%, 26%, 28%, or 30% oxidized species. For example, a composition provided herein that is exposed to light at 1600-1800 lux for 5 days has no more than about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12%, 14%, 16%, 18%, 20%, 22%, 24%, 26%, 28%, or 30% oxidized species selected from the group consisting of: M257 of SEQ ID NO: 10, and M433 of SEQ ID NO: 10. For example, a composition provided herein that is exposed to light at 1600-1800 lux for 5 days has no more than about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12%, 14%, 16%, 18%, 20%, 22%, 24%, 26%, 28%, or 30% oxidized species selected from the group consisting of: M257 of SEQ ID NO: 10, M433 of SEQ ID NO: 10, W102 of SEQ ID NO: 10, and W104 of SEQ ID NO: 10. For example, a composition provided herein that is exposed to light at 1600-1800 lux for 3 to 5 days has no more than about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12%, 14%, 16%, 18%, 20%, 22%, 24%, 26%, 28%, or 30% oxidized species selected from the group consisting of: M257 of SEQ ID NO: 10, M433 of SEQ ID NO: 10, W102 of SEQ ID NO: 10, W104 of SEQ ID NO: 10, and combinations thereof.

In embodiments, utilization of provided methods is effective at stabilizing a level of post-translational modification of a residue of a CDR in compositions of the disclosure. The various provided methods can be useful at maintaining PTM levels of provided compositions (e.g., compositions comprising sequences of Table 1 or compositions with at least about 85% identity to those of Table 1) throughout a manufacturing process and/or throughout transport and subject dosing. Maintenance of PTM can be useful for preserving potency of provided compositions. For example, compositions of the disclosure can have a stable level of PTM, of one or more residues, following light exposure. For example, a composition provided herein that is exposed to light at 1600-1800 lux for 3 days has no more than about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12%, 14%, 16%, 18%, 20%, 22%, 24%, 26%, 28%, or 30% modified species. For example, a composition provided herein that is exposed to light at 1600-1800 lux for 4 days has no more than about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12%, 14%, 16%, 18%, 20%, 22%, 24%, 26%, 28%, or 30% modified species. For example, a composition provided herein that is exposed to light at 1600-1800 lux for 5 days has no more than about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12%, 14%, 16%, 18%, 20%, 22%, 24%, 26%, 28%, or 30% modified species. For example, a composition provided herein that is exposed to light at 1600-1800 lux for 5 days has no more than about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12%, 14%, 16%, 18%, 20%, 22%, 24%, 26%, 28%, or 30% modified species selected from the group consisting of: M257 of SEQ ID NO: 10, and M433 of SEQ ID NO: 10. For example, a composition provided herein that is exposed to light at 1600-1800 lux for 5 days has no more than about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12%, 14%, 16%, 18%, 20%, 22%, 24%, 26%, 28%, or 30% modified species selected from the group consisting of: M257 of SEQ ID NO: 10, M433 of SEQ ID NO: 10, W102 of SEQ ID NO: 10, and W104 of SEQ ID NO: 10. For example, a composition provided herein that is exposed to light at 1600-1800 lux for 3 to 5 days has no more than about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12%, 14%, 16%, 18%, 20%, 22%, 24%, 26%, 28%, or 30% modified species selected from the group consisting of: M257 of SEQ ID NO: 10, M433 of SEQ ID NO: 10, W102 of SEQ ID NO: 10, W104 of SEQ ID NO: 10, and combinations thereof. In embodiments, compositions bind ILT7 and comprise the Complementarity-Determining Regions (CDRs) HCDR1, HDR2, HCDR3, LCDR1, LCDR2, and LCDR3 comprising the sequences of SEQ ID NOs: 3, 4, 5, 6, 7, and 8, respectively.

In embodiments, methods described herein are effective at generating Fc oxidation resistant compositions. For example, utilization of any of the compositions and methods of the disclosure can prevent oxidation of vulnerable residues as compared to oxidation in otherwise comparable compositions absent the methods or compositions of the disclosure. In embodiments, the vulnerable residues are M257 and/or M433 of SEQ ID NO: 10.

In embodiments, a composition provided herein that comprises L-methionine (e.g., about 15 mM L-methionine) has a reduced percentage of oxidized species. In embodiments, a composition provided herein exposed to light at 1600-1800 lux from about 3 to about 5 days has a lower percentage of oxidized species as compared to an otherwise comparable composition lacking a formulation provided herein and/or lacking L-methionine (e.g., about 15 mM L-methionine). In embodiments, the percentage of oxidized species is reduced by about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 45%, 50%, 55%, 60%, 65%, 70%, 75%, 80%, 85%, 90%, 95%, or up to about 100% as compared to an otherwise comparable composition lacking a formulation provided herein and/or lacking L-methionine. In embodiments, when a composition provided herein is exposed to light at 1600-1800 lux for about 5 days a percentage of oxidized species is at a range from 0.2% to about 0.5%. In embodiments, the percentage of oxidized species in a disclosed composition is less than 0.5% when exposed to light at 1600-1800 lux for 1, 2, 3, 4, 5, 6, 7, 8, 9, or up to 10 days. In embodiments, the percentage of oxidized species in a disclosed composition is less than 0.4% when exposed to light at 1600-1800 lux for 1, 2, 3, 4, 5, 6, 7, 8, 9, or up to 10 days. In embodiments, the percentage of oxidized species in a disclosed composition is less than 0.3% when exposed to light at 1600-1800 lux for 1, 2, 3, 4, 5, 6, 7, 8, 9, or up to 10 days. In embodiments, the percentage of oxidized species in a disclosed composition is less than 0.2% when exposed to light at 1600-1800 lux for 1, 2, 3, 4, 5, 6, 7, 8, 9, or up to 10 days. In embodiments, the percentage of oxidized species in a disclosed composition is less than 0.1% when exposed to light at 1600-1800 lux for 1, 2, 3, 4, 5, 6, 7, 8, 9, or up to 10 days.

In embodiments, a composition can be Fc oxidation resistant or have reduced Fc oxidation of vulnerable residues. Reduction of Fc oxidation can refer to a slowing or reduced rate of oxidation when exposed to degrative elements such as light. For example, when compositions of the disclosure are exposed to light at 1600-1800 lux for 3 to 5 days, the percentage of oxidized species of ILT7 binding protein in the composition is merely increased at a rate of 0.2%-0.5% per day. In embodiments, when a composition comprising a sequence of Table 1, or a sequence having at least about 85% identity thereto, are exposed to light at 1600-1800 lux for 3 to 5 days, the percentage of oxidized species in the composition is increased at a rate of at most about 0.2% per day. In embodiments, when a composition comprising a sequence of Table 1, or a sequence having at least about 85% identity thereto, are exposed to light at 1600-1800 lux for 3 to 5 days, the percentage of oxidized species in the composition is increased at a rate of at most about 0.3% per day. In embodiments, when a composition comprising a sequence of Table 1, or a sequence having at least about 85% identity thereto, are exposed to light at 1600-1800 lux for 3 to 5 days, the percentage of oxidized species in the composition is increased at a rate of at most about 0.4% per day. In embodiments, when a composition comprising a sequence of Table 1, or a sequence having at least about 85% identity thereto, are exposed to light at 1600-1800 lux for 3 to 5 days, the percentage of oxidized species in the composition is increased at a rate of at most about 0.5% per day. In embodiments, compositions bind ILT7 and comprise the Complementarity-Determining Regions (CDRs) HCDR1, HDR2, HCDR3, LCDR1, LCDR2, and LCDR3 comprising the sequences of SEQ ID NOs: 3, 4, 5, 6, 7, and 8, respectively.

In embodiments, an about 15 mM concentration of L-methionine is effective in reducing aggregation or a rate thereof of a composition provided herein as compared to an otherwise comparable composition lacking a formulation provided herein and/or lacking L-methionine (e.g., less than 15 mM L-methionine). In embodiments, about 15 mM concentration of L-methionine is effective in reducing aggregation of a composition provided herein as compared to an otherwise comparable composition lacking L-methionine (e.g., less than 15 mM L-methionine) by at least about 1-fold, 5-fold, 10-fold, 15-fold, 25-fold, 50-fold, 75-fold, 100-fold, 150-fold, or 250-fold. In embodiments, an about 15 mM concentration of L-methionine is effective in reducing aggregation, of a composition provided herein under stressed conditions (e.g., 40° C.) as compared to an otherwise comparable composition lacking L-methionine (e.g., less than 15 mM L-methionine). In embodiments, an about 15 mM concentration of L-methionine is effective in reducing aggregation, of a composition provided herein under stressed conditions (e.g., 40° C.) as compared to an otherwise comparable composition lacking L-methionine (e.g., less than 15 mM L-methionine) by at least about 1-fold, 5-fold, 10-fold, or 15-fold.

In embodiments, an about 15 mM concentration of L-methionine is effective in reducing aggregation, reducing oxidation, or both reducing aggregation and reducing oxidation of a composition provided herein as compared to an otherwise comparable composition at a different concentration of L-methionine by at least about: 0.1%, 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 45%, 50%, 55%, 60%, 65%, 70%, 75%, 80%, 85%, 90%, 95%, or up to about 100% as compared to an otherwise comparable composition lacking a formulation provided herein and/or lacking L-methionine (e.g., about 15 mM L-methionine). In embodiments, oxidation can be determined using any assay of the art (e.g., peptide mapping, reverse-phase HPLC (RP-HPLC), LC-MS methods, e.g., electrospray ionization (ESI) MS, etc.). In embodiments, a rate of oxidation is determined. In embodiments, the presence or absence of oxidation is determined. In embodiments, oxidation is determined at a variety of temperatures. In embodiments, oxidation is determined under stressed conditions (e.g., 40° C.). In embodiments, oxidation is determined at room temperature. In embodiments, oxidation is determined at 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C.

Aggregate and particle formation can be determined using any assay in the art. In embodiments, aggregate and particle formation is determined using an in vitro assay including but not limited to Size-Exclusion Chromatography (SEC), Micro-Flow Imaging (MFI) and combinations thereof. In embodiments, aggregate and particle formation is determined using SEC. In embodiments, aggregate and particle formation is determined using MFI.

In embodiments, a 15 mM concentration of L-methionine is effective in reducing a color change of a composition provided herein as compared to an otherwise comparable composition lacking the L-methionine, lacking the about 10-20 mM of L-methionine, or lacking the about 15 mM of L-methionine. In embodiments, a color change can result from stress conditions such as increased temperature, agitation, increased pressure, or any combination thereof.

In embodiments, a composition is colorless. In embodiments, a composition is clear. In embodiments, a composition is free from visible particles. In embodiments, a composition can be opalescent. In embodiments, color change can be determined using any means including but not limited to visual inspection, microscopy, colorimetry, turbidimetry, and combinations thereof.

In embodiments, a composition comprising an anti-ILT7 binding protein is more stable in a histidine buffer as compared to an otherwise comparable composition in a different buffer. In embodiments, a composition comprising an anti-ILT7 binding protein is more stable in a histidine buffer as compared to an otherwise comparable composition in phosphate buffer or citrate buffer. In embodiments, a histidine buffer is effective in reducing aggregation, reducing oxidation, or both reducing aggregation and reducing oxidation. In embodiments, the reduced oxidation can refer to reduced oxidation of L-methionine. In embodiments, the reduced oxidation can refer to reduced oxidation of a residue of L-methionine including but not limited to W102 and/or W104 of SEQ ID NO: 1.

In embodiments, a composition comprising an anti-ILT7 binding protein is more stable in a histidine buffer as compared to an otherwise comparable composition in a different buffer by at least about 1-fold, 5-fold, 10-fold, 15-fold, 25-fold, 50-fold, 75-fold, 100-fold, 150-fold, or 250-fold. In embodiments, a composition comprising an anti-ILT7 binding protein is more stable in a histidine buffer as compared to an otherwise comparable composition in a different buffer by at least about 1-fold, 5-fold, 10-fold, 15-fold, 25-fold, 50-fold, 75-fold, 100-fold, 150-fold, or 250-fold. In embodiments, a composition comprising an anti-ILT7 binding protein is more stable in a histidine buffer as compared to an otherwise comparable composition in a different buffer by at least about 1-fold, 5-fold, or 10-fold. In embodiments, a histidine buffer is effective in reducing aggregation in a composition comprising an anti-ILT7 binding protein as compared to an otherwise comparable composition in a different buffer by at least about 1-fold, 5-fold, 10-fold, 15-fold, 25-fold, 50-fold, 75-fold, 100-fold, 150-fold, or 250-fold. In embodiments, a histidine buffer is effective in reducing aggregation in a composition comprising an anti-ILT7 binding protein as compared to an otherwise comparable composition in a different buffer by at least about 1-fold, 5-fold, or 10-fold.

Stability of an anti-ILT7 binding protein can be evaluated using any means in the art. In embodiments, evaluating stability can comprise determining any one of: specificity, binding affinity, purity, aggregation, degradation, and thermal stability. In embodiments, stability is determined by way of: ELISA, Western blotting and affinity analysis, SPR/BIACORE™, all of which can be utilized to indicate the retention of biological function (e.g., antigen binding, specificity, affinity, percent monomeric) during conditions encountered during production/isolation and following storage. In addition, circular dichroism (CD), nuclear magnetic resonance (NMR) and mass spectroscopy can be used to probe antibody stability. In embodiments, stability is determined by an assay selected from the group consisting of: size exclusion chromatography (e.g., SEC-HPLC, SEC-FPLC), ELISA, western blot, BIACORE™, SDS-PAGE, dynamic light scattering (DLS), differential scanning calorimetry (DSC), analytical ultracentrifugation (AUC), differential scanning fluorimetry, and combinations thereof.

In embodiments, stability is determined. In embodiments, stability is determined at a variety of temperatures. In embodiments, stability is determined under stressed conditions (e.g., 40° C.). In embodiments, stability is determined at room temperature. In embodiments, stability is determined at 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C.

In embodiments, an anti-ILT7 binding protein or composition comprising the same comprises an increased shelf-life. In embodiments, an anti-ILT7 binding protein or composition comprising the same comprises an increased shelf-life as compared to an otherwise comparable composition lacking a histidine buffer. In embodiments, a shelf-life is increased by at least about 1 month, 2 months, 4 months, 6 months, 8 months, 10 months, 12 months, 14 months, 16 months, 18 months, 20 months, 22 months, 24 months, 26 months, 28 months, 30 months, 32 months, 34 months, 36 months, 38 months, 40 months, 42 months, 44 months, 46 months, 48 months, 50 months, 52 months, 54 months, 56 months, 58 months, 60 months, 62 months, 64 months, 66 months, 68 months, 70 months, 72 months, 74 months, 76 months, 78 months, 80 months, 82 months, 84 months, 86 months, 88 months, 90 months, 92 months, 94 months, 96 months, 98 months, or 100 months. In embodiments, a composition provided herein has a shelf-life of about 60 months. In embodiments, a composition provided herein has a shelf-life of about 36 months. In embodiments, a shelf-life can be at a temperature of about 2° C. to about 8° C.

In embodiments, a histidine buffer is effective in reducing pain upon injection of a composition to a subject in need thereof. In embodiments, a histidine buffer is effective in reducing pain upon injection of a composition to a subject in need thereof as compared to an otherwise comparable composition using a different buffer (e.g., a citrate buffer). In embodiments, a histidine buffer is effective in reducing pain associated with a subcutaneous injection of a composition provided herein (e.g., a composition comprising an anti-ILT7 binding protein). In embodiments, a subject can assess pain by way of a scale selected from the group consisting of: numerical rating scale, visual analog scale, adult non-verbal pain scale, behavioral pain scale, critical-care observation tool, Defense and Veterans Pain Rating Scale, Pain Assessment in Advanced Dementia Scale, interview, or any combination thereof. In embodiments, a histidine buffer is effective in reducing pain associated with administration of a composition provided herein by at least about 1, 2, 3, 4, 5, 6, 7, 8, 9, or 10 points on a scale. In embodiments, a histidine buffer is effective in reducing pain associated with administration of a composition provided herein by at least about 1-2 points, 1-3 points, or 1-4 points.

Viscosity can be determined using any means. In embodiments, viscosity is determined using a viscosity modifier (e.g., arginine, NaCl). In embodiments, viscosity can be determined using a viscometer.

Alternate and supplemental methods of reducing and eliminating degradative modification of amino acid residues (e.g., oxidation) of described compositions are also contemplated. For example, the methods of purification described in PCT/US2016/022003, which is incorporated by reference in its entirety, are also contemplated. In embodiments, the provided methods can be combined with the purification methods of PCT/US2016/022003.

### Treatment

Provided herein are also methods for using a composition comprising an anti-ILT7 binding protein. In embodiments, methods of treating a subject with an autoimmune disease comprising administering to the subject an effective amount of an anti-ILT7 binding protein are provided herein. Also provided are methods of treating a subject in need comprising a disease or condition associated with an ILT7 expressing cell. Also provided are methods of treatment comprising a subcutaneous administration of a disclosed composition via autoinjector.

In embodiments, treatment comprises administration of an anti-ILT7 binding protein as part of a method described herein to a subject in need thereof or to a subject that is suspected of needing treatment thereof. In embodiments, administration of an anti-ILT7 binding protein as part of a method described herein isolates a tissue, a cell, or cells from a subject, where the subject has a disease, a symptom of a disease, or a predisposition toward a disease (e.g., an autoimmune disease). In embodiments, a subject in need thereof or a subject that is suspected of needing treatment thereof may have high pDC numbers or activity as compared to a subject not in need thereof. In embodiments, a subject in need thereof or a subject that is suspected of needing treatment thereof even though does not have a formal diagnosis, e.g., the subject may or may not have systemic lupus erythematosus (SLE) or cutaneous lupus erythematosus (CLE), but exhibits symptoms, e.g., high pDC numbers or activity as compared to a subject not in need thereof. In embodiments, a subject suspected of needing treating has a high baseline blood type I IFNGS level. In embodiments, a treatment comprises administration of a pharmaceutical composition comprising an anti-ILT7 binding protein used in a method described herein to a subject in need thereof or to a subject that is suspected of needing treatment thereof. In embodiments, a treatment comprises administration of a pharmaceutical composition comprising an anti-ILT7 binding protein as part of a method described herein to an isolate a tissue, a cell, or cells from a subject who has a disease, a symptom of a disease, or a predisposition toward a disease (e.g., an autoimmune disease).

Examples of autoimmune disorders that may be treated when the subject is exhibiting elevated type I IFNGS include but are not limited to discoid lupus erythematosus (DLE), systemic lupus erythematosus (SLE), lupus nephritis, cutaneous lupus erythematosus (CLE), Sjögren's syndrome, inflammatory myositis (e.g., dermatomyositis, inclusion body myositis, juvenile myositis and polymyositis), systemic sclerosis, diabetes, focal segmental glomerulosclerosis (FSGS), alopecia areata, Hashimoto's disease, autoimmune adrenal insufficiency, pure red cell anemia, multiple sclerosis, rheumatic carditis, IgG4RD, psoriasis, psoriatic arthritis, rheumatoid arthritis, chronic inflammation, chronic rheumatism, vitiligo, alopecia areata, hidradenitis suppurativa, celiac disease, acute graft versus host disease (GVHD), chronic GVHD, vascular inflammation, myocardial infarction, and Type-1 interferonopathies. In embodiments, an autoimmune disease is DLE. In embodiments, an autoimmune disease is SLE. In embodiments, an autoimmune disease is CLE. In embodiments, an autoimmune disease is lupus, but is not DLE. In embodiments, an autoimmune disease is Sjögren's syndrome. In embodiments, an autoimmune disease is dermatomyositis. In embodiments, an autoimmune disease is polymyositis. In embodiments, an autoimmune disease is systemic sclerosis. In embodiments, an autoimmune disease is hidradenitis suppurativa. In embodiments, an autoimmune disease is vitiligo. In embodiments, an autoimmune disease comprises FSGS. In embodiments, an autoimmune disease comprises alopecia areata.

Without having to be bound by theory, an anti-ILT7 binding protein as part of a method described herein can induce antibody-dependent cell-mediated cytotoxicity (ADCC) activity against plasmacytoid dendritic cells (pDCs), thereby reducing or depleting pDCs. In embodiments, anti-ILT7 binding protein-mediated ADCC causes a reduction in circulating pDCs. In embodiments, anti-ILT7 binding protein-mediated ADCC causes a reduction in local or tissue pDCs. In embodiments, a tissue in which the pDCs are reduced includes but is not limited to skin cells, skin biopsy samples, kidney cells, lung cells, liver cells, heart cells, brain cells, nervous tissue, thyroid cells, eye cells, skeletal muscle cells, cartilage, bone tissue, and cells from airway passages. In embodiments, a tissue is a skin biopsy sample. In embodiments, administering an anti-ILT7 binding protein will cause a reduction in pDCs. In embodiments, administering an anti-ILT7 binding protein will cause a reduction in skin pDCs. Normally, pDCs are not present in skin tissue, and immature pDCs are typically only found in blood, thymus lymphoid tissue, tonsils, and lung tissue. Thus, the presence of pDCs in skin biopsy samples is indicative of an abnormal condition in which pDCs are recruited to the skin. In embodiments, a method of the present disclosure comprises administering an anti-ILT7 binding protein to a subject in need of treatment of a condition marked by the presence of pDCs in the subject's skin. In embodiments, a method of the present disclosure comprises reducing pDC levels in a subject's skin by administering an anti-ILT7 binding protein to the subject in need of treatment thereof.

In embodiments, reduction in pDC levels in a subject is about 1% to about 100% as compared to the pDC levels in a subject prior to administration of an anti-ILT7 binding protein, i.e. a baseline level of a subject. In embodiments, a reduction in pDC levels in a subject is at least about 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 45%, 50%, 55%, 60%, 65%, 70%, 75%, 80%, 85%, 90%, 95%, or up to about 100% compared to pDCs levels a baseline level of a subject. In embodiments, a reduction in pDC levels in a subject is at least about 50% compared to pDC levels in a baseline level of a subject. In embodiments, administration of a therapeutically effective amount of an anti-ILT7 binding protein leads to at least about 10% reduction in total number of pDCs in a subject. In embodiments, administration of a therapeutically effective amount of an anti-ILT7 binding protein leads to at least about 10% reduction in activated pDCs in a subject. In embodiments, pDC levels are measured in a test biological sample taken from a subject. In embodiments, administration of a therapeutically effective amount of an anti-ILT7 binding protein as part of a method described herein to a subject in need thereof leads to a reduction in pDC levels in a test biological sample taken from a subject. In embodiments, a reduction in pDC levels in a test biological sample taken from the subject is at least about 10% compared to pDC levels in the test biological sample prior to administration of an anti-ILT7-binding protein used as part of a method described herein. Whole blood, plasma, urine, saliva, tissue, cell(s), and serum samples may be collected as a test biological sample to evaluate pDC levels. In embodiments, a test biological sample is whole blood. In embodiments, a test biological sample is tissue, including, but not limited to, skin cells and skin biopsy specimens. In embodiments, the pDCs are circulating pDCs. In embodiments, the pDCs are pDCs in the skin. In embodiments, the reduction in pDCs is reversible.

In embodiments, a subject can have elevated or high levels of pDCs in skin tissue prior to a treatment. In embodiments, a subject with a high pDC level in skin tissue prior to a treatment is more responsive to a treatment. In embodiments, a subject with a high pDC level in skin tissue has a pDC level of at least about 50 pDC/mm² of skin tissue, at least about 60 pDC/mm² of skin tissue, at least about 70 pDC/mm² of skin tissue, at least about 80 pDC/mm² of skin tissue, at least about 90 pDC/mm² of skin tissue, at least about 100 pDC/mm² of skin tissue, at least about 110 pDC/mm² of skin tissue, at least about 120 pDC/mm² of skin tissue, at least about 125 pDC/mm² of skin tissue, at least about 150 pDC/mm² of skin tissue, at least about 175 pDC/mm² of skin tissue, at least about 200 pDC/mm² of skin tissue, or higher. In embodiments, a low pDC level in skin tissue is considered less than about 10 pDC/mm² of skin tissue. In embodiments, a high pDC level in skin tissue is considered at least about 100 pDC/mm² of skin tissue.

In embodiments, administration of a therapeutically effective amount of an anti-ILT7 binding protein used as part of a method described herein to a subject in need thereof causes at least about 10% reduction in pDCs in the subject at about 3 hours, about 6 hours, about 12 hours, about 24 hours, or about 48 hours following administration of the anti-ILT7 binding protein. In embodiments, administration of a therapeutically effective amount of an anti-ILT7 binding protein as part of a method described herein to a subject in need thereof causes an at least about 10% reduction in pDC levels in a test biological sample taken from the subject at about 5 minutes, at about 10 minutes, about 15 minutes, about 30 minutes, about 1 hour, about 2 hours, about 3 hours, about 6 hours, about 12 hours, about 24 hours, or about 48 hours following administration of the anti-ILT7 binding protein.

In embodiments, reduction in pDC levels persists for at least about 1 day, at least about 2 days, at least about 3 days, at least about 4 days, at least about 5 days, at least about 6 days, at least about 7 days, at least about 14 days, at least about 21 days, at least about 28 days, at least about 30 days, at least about 45 days, at least about 60 days, at least about 90 days, or at least about 180 days or longer following administration of a therapeutically effective amount of an anti-ILT7 binding protein as part of a method described herein to a subject in need thereof. In embodiments, reduction in pDC levels persist for at least about 30 days following administration of a therapeutically effective amount of an anti-ILT7 binding protein as part of a method described herein to a subject in need thereof. In embodiments, reduction in pDC levels persist for at least about 60 days following administration of a therapeutically effective amount of an anti-ILT7 binding protein used as part of a method described herein to a subject in need thereof.

In embodiments, a method of the present disclosure can be used to monitor effectiveness of treatment by monitoring a type I interferon gene signature (IFNGS) and/or pDCs levels. As noted above, autoimmune conditions are often marked by an elevated type I IFNGS and/or elevated pDCs. In embodiments, monitoring effectiveness of treatments administered to a subject in need thereof (e.g., has a diagnosis and/or symptoms of an autoimmune disease or condition) can comprise monitoring a type I IFNGS and/or pDC levels.

In embodiments, the disclosure provides a method of monitoring effectiveness of treatment of an autoimmune disorder or condition, comprising: (a) measuring a type I IFNGS in a biological sample taken from a subject to obtain a baseline value of a type I IFNGS; and (b) measuring a type I IFNGS in a biological sample taken from a subject after administering a treatment, wherein the treatment comprises administering an anti-ILT7 binding protein, and wherein a decrease in the type I IFNGS in (b) compared to the baseline value in (a) indicates that the treatment is effective in the subject.

In embodiments, a treatment results in a decrease in a type I IFNGS compared to a baseline value. In embodiments, a decrease in a type I IFNGS compared to a baseline value ranges from about 1% to about 99%. In embodiments, a decrease in type I IFNGS levels compared to a baseline value is at least about 5%, at least about 10%, at least about 20%, at least about 30%, at least about 40%, at least about 50%, at least about 60%, at least about 70%, at least about 80%, at least about 90%, at least about 95%, or at least about 99%. In embodiments, a decrease in type I IFNGS levels compared to a baseline value is at least about 30%. In embodiments, a decrease in type I IFNGS levels compared to a baseline value is at least about 50%.

In embodiments, an elevation in type I IFNGS in a test biological sample relative to a normal biological sample, or in a subject in need thereof relative to a normal subject, is at least a fold change of about 1.1 to about 1000. In embodiments, a type I IFNGS is elevated by at least about 1.1, 1.2, 1.3, 1.4, 1.5, 2, 3, 4, 5, 6, 7, 8, 9, 10, 15, 20, 30, 40, 50, 60, 70, 80, 90, or 100-fold in a test biological sample relative to a normal biological sample, or in a subject in need of thereof relative to a normal subject. In embodiments, type I IFNGS levels are elevated by at least about 4-fold in the test biological sample relative to the normal biological sample, or in a subject in need of thereof relative to a normal subject.

In embodiments, a reduction in type I IFNGS persists for at least about 1 day, at least about 2 days, at least about 3 days, at least about 4 days, at least about 5 days, at least about 6 days, at least about 7 days, at least about 14 days, at least about 21 days, at least about 28 days, at least about 30 days, at least about 45 days, at least about 60 days, at least about 90 days, or at least about 180 days or longer following administration of a therapeutically effective amount of an anti-ILT7 binding protein as part of a method described herein to a subject in need thereof. In embodiments, a reduction in type I IFNGS persists for up to about 30 days following administration of a therapeutically effective amount of an anti-ILT7 binding protein as part of a method described herein to a subject in need thereof. In embodiments, a reduction in type I IFNGS persists for up to about 60 days following administration of a therapeutically effective amount of an anti-ILT7 binding protein as part of a method described herein to a subject in need thereof.

In embodiments, a method of the present disclosure comprises administering an anti-ILT7 binding protein as part of a method described herein to suppress release of type I IFN from pDCs, regardless of the location of the pDCs. In embodiments, a method of the present disclosure comprises administering an anti-ILT7 binding protein to suppress release of type I IFN from pDCs in blood or circulation. In embodiments, a method of the present disclosure comprises administering an anti-ILT7 binding protein to suppress release of type I IFN from local pDCs. In embodiments, a method of the present disclosure comprises administering an anti-ILT7 binding protein to suppress release of type I IFN from pDCs in the skin of a subject. In embodiments, the suppressed type I IFN is IFNα. In embodiments, an anti-ILT7 binding protein-mediated suppression of release of type I IFN from pDCs causes a reduction in type I IFNGS.

The disclosure provides methods for treating an autoimmune disorder in a subject with an anti-ILT7 binding protein. In embodiments, a method comprises treating an autoimmune disorder in a subject in need thereof, wherein the subject is determined to have a high blood type I interferon gene signature (IFNGS) level. In embodiments, the disclosure provides a method for reducing the IFNGS in a subject in need thereof. In embodiments, a method comprises administering to a subject a pharmaceutically effective amount of an anti-ILT7 binding protein. In embodiments, an anti-ILT7 binding protein is administered to a subject when a type I IFNGS is elevated in a subject relative to a type I IFNGS in a normal subject. In embodiments, for example, an anti-ILT7 binding protein is administered to a subject with an elevated baseline type I IFNGS relative to a type I IFNGS in a normal subject. In embodiments, a method comprises selecting a subject for treatment with an anti-ILT7 binding protein, the method comprising: (i) determining a baseline blood type I IFNGS level of a subject, and (ii) selecting one or more subjects with high baseline blood type I IFNGS levels for treatment with the ILT7 binding protein. In embodiments, the anti-ILT7 binding protein is an antibody specific for ILT7 protein.

In certain diseases (e.g., autoimmune diseases), activated pDCs secrete significant amounts of type I and type III interferons (IFNs). Type I IFNs are a large group of IFN proteins that help regulate the immune system. The mammalian IFNs are designated IFNα, PTNIb, IFNco, IFNK, IFNT, IFN6, IPMz, and IFNu. In embodiments, a type I IFN that generates the type I IFNGS is IFNα. Type I IFN protein levels cannot be directly measured in a reliable way; however, measurement of IFN-inducible genes serves as a robust surrogate to Type 1 IFN protein levels. In embodiments, expression levels of type I IFN-inducible genes can be measured in a biological sample (e.g., blood, skin, skeletal muscles, etc.) and analyzed as a composite outcome referred to as the “type I interferon gene signature” or “type I IFNGS” or “IFNGS.”

In embodiments, a type I IFNGS is determined by assaying expression levels of at least 2, at least 5, at least 10, at least 15, at least 20, at least 25, at least 30, at least 40, at least 50, at least 60, at least 70, at least 80, at least 90, at least 100, at least 200, at least 300, at least 400, or at least 500 type I IFN-inducible genes in a biological sample. In embodiments, determination of a type I IFNGS comprises the collective expression levels of two or more type I IFN-inducible genes. In embodiments, the two or more type I interferon (IFN)-inducible genes include but are not limited to two or more genes chosen from SPATS2L, EPSTI1, HERC5, IF 127, IFI44, IFI44L, IFI6, IFIT1, IFIT3, ISG15, LAMP3, LY6E, MX1, OAS1, OAS2, OAS3, PLSCR1, RSAD2, RTP4, SIGLEC1, and USP18. In embodiments, a type I IFNGS is determined by assaying the collective expression levels of SPATS2L, EPSTI1, HERC5, IFI27, IFI44, IFI44L, IFI6, IFIT1, IFIT3, ISG15, LAMP3, LY6E, MX1, OAS1, OAS2, OAS3, PLSCR1, RSAD2, RTP4, SIGLEC1, and USP18. These gene symbols are well-known in the art and refer to human and non-human orthologs of the listed genes.

In embodiments, a type I IFNGS is determined by assaying a collective expression of genes, wherein the collective expression comprises a 21-gene signature. In embodiments, a type I IFNGS in a subject is at elevated by at least 1.5-fold relative to a normal score prior to a treatment. In embodiments, a type I IFNGS in a subject is elevated by at least 2-fold relative to a normal score prior to a treatment. In embodiments, a subject with an elevated type I IFNGS prior to treatment are more responsive to a treatment. In embodiments, a type I IFNGS is at least about 4-fold, at least about 5-fold, at least about 6-fold, at least about 7-fold, at least about 8-fold, at least about 9-fold, at least about 10-fold, at least about 11-fold, at least about 12-fold or higher relative to a normal score prior to treatment with an anti-ILT7 binding protein used as part of a method described herein. In embodiments, a tissue type I IFNGS is determined from a skin biopsy. In embodiments, a tissue type I IFNGS is determined using an IFN-inducible Myxovirus protein A (MxA) immunohistochemistry (IHC) test.

As used herein, the term “high” or “elevated” when used in conjunction with IFGNS means that a type I IFNGS is a fold change of at least about 1.1 to about 1000 compared to normal type I IFNGS levels. By “normal type I IFNGS” is intended a type I IFNGS obtained from a normal subject. The terms “high” or “elevated” when used in conjunction with type I IFNGS are used interchangeably. In embodiments, a type I IFNGS is “high” or “elevated” when a type I IFNGS used as part of a method described herein is at least about 1.1, 1.2, 1.3, 1.4, 1.5, 2, 3, 4, 5, 6, 7, 8, 9, 10, 15, 20, 30, 40, 50, 60, 70, 80, 90, or 100-fold greater relative to a type I IFNGS in a normal subject. In embodiments, a method of treatment described herein is applied when a type I IFNGS is elevated by at least about 4-fold relative to a normal type I IFNGS.

The disclosure provides a method of treating a subject with elevated type I IFNGS comprising administering an anti-ILT7 binding proteins described herein. A subject may exhibit an elevated type I IFNGS when suffering from an autoimmune disorder. In embodiments, the present disclosure provides a method of treating an autoimmune disorder when a subject is exhibiting an elevated type I IFNGS. In embodiments, an autoimmune disorder is otherwise asymptomatic. In embodiments, a method comprises selecting a subject for treatment with an anti-ILT7-binding protein, the method comprising: (i) determining baseline blood type I IFNGS levels of a subject, and (ii) selecting one or more subjects with high baseline blood type I IFNGS levels for treatment with an ILT7-binding protein.

In embodiments, a method of the present disclosure can be used to monitor effectiveness of treatment of conditions or disorders by monitoring levels of type I IFNGS and/or activated pDCs. As noted above, autoimmune conditions are often marked by elevated type I IFNGS and/or elevated pDCs, thus monitoring the effectiveness of treatments can include monitoring type I IFNGS and/or pDC levels. In embodiments, the disclosure provides a method of monitoring effectiveness of treatment of an autoimmune disorder or condition, comprising: (a) measuring a type I interferon gene signature (IFNGS) in a biological sample taken from a subject to obtain a baseline value of the type I IFNGS; and (b) measuring a type I IFNGS in a biological sample taken from a subject after administering a treatment, wherein the treatment comprises administering an anti-ILT7-binding protein, and wherein a decrease in the type I IFNGS in step (b) compared to the baseline value (a) indicates that the treatment is effective in the subject. In embodiments, a treatment results in a decrease in a type I IFNGS compared to a baseline value. In embodiments, a decrease in a type I IFNGS compared to a baseline value ranges from about 1% to about 99%. In embodiments, a decrease in a type I IFNGS compared to a baseline value is at least about 5%, at least about 10%, at least about 20%, at least about 30%, at least about 40%, at least about 50%, at least about 60%, at least about 70%, at least about 80%, at least about 90%, at least about 95%, or at least about 99%. In embodiments, a decrease in a type I IFNGS compared to a baseline value is at least about 30%. In embodiments, a decrease in a type I IFNGS compared to a baseline value is at least about 50%.

In embodiments, a method of the present disclosure can comprise reducing Cutaneous Lupus Erythematosus Disease Activity and Severity Index (CLASI) in a tissue of a subject in need thereof. A method of the present disclosure can comprise administering to a subject a pharmaceutically effective amount of an anti-ILT7 binding protein. As used herein, the term “CLASI” refers to Cutaneous Lupus Erythematosus Disease Activity and Severity Index. The CLASI is a validated instrument for measuring skin manifestations of CLE. The CLASI consists of two scores: the first summarizes the inflammatory activity of the disease; the second is a measure of the damage done by the disease. An activity score includes erythema (0-3), scale/hypertrophy (0-2), mucous membrane lesions (0-1), recent hair loss (0-1) and non-scarring alopecia (0-3). A damage score includes dyspigmentation (0-1), scarring/atrophy/panniculitis (0-2), and scarring of the scalp (0-6). Subjects are asked if their dyspigmentation lasts 12 months or longer, in which case, the dyspigmentation score is doubled. Each of the above parameters is measured in 13 different anatomical locations, included specifically because they are most often involved in CLE. The most severe lesion in each area is measured. As used herein, the term “reduction CLASI” refers diminished levels of CLASI-Activity (CLASI-A) score in a subject or in a biological sample (e.g., skin tissues, skin cells, skin biopsy samples, etc.) taken from the subject, or diminished levels of CLASI-Damage (CLASI-D) score in a subject or in a biological sample taken from the subject, or both. In embodiments, a method of the disclosure results in a reduced CLASI-A score in a subject. In embodiments, a reduction in a CLASI-A score of a subject involves a reduction of the CLASI-A score by at least 1, 2, 3, 4, 5, 6, 7, 8, 9, or 10 points from a baseline value. In embodiments, a reduction in a CLASI-A score of a subject involves a reduction of the CLASI-A score by at least 4 points from a baseline value. In embodiments, a reduction in a CLASI-A score of a subject involves a reduction of the CLASI-A score by at least 7 points from a baseline value. In embodiments, a reduction in the CLASI-A score of a subject involves a reduction of the CLASI-A score by at least 5%, 10%, 15%, 20%, 25%, 30%, 35%, 40%, 45%, 50%, 55%, 60%, 65%, 70%, 75%, 80%, 85%, 90%, 95%, or 99% from a baseline value. In embodiments, a reduction in the CLASI-A score of a subject involves a reduction of the CLASI-A score by at least 50% from a baseline value. In embodiments, a baseline value is a value of a CLASI-A score in a subject prior to treatment with an anti-ILT7 binding protein as part of a method described herein. In embodiments, a method of the present disclosure results in a reduced CLASI-D score in a subject. In embodiments, a method of the present disclosure result in a reduced CLASI-A score and a reduced CLASI-D score in a subject.

Disclosed compositions can be administered using a variety of regimens. In embodiments, a composition is administered hourly, daily, weekly, monthly, or yearly.

In embodiments, a pharmaceutical composition provided herein can be administered hourly. In embodiments, a pharmaceutical composition is administered every 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. In embodiments, a pharmaceutical composition is administered every day, 2 days, 3 days, 4 days, 5 days, 6 days, or 7 days. In embodiments, a pharmaceutical composition is administered every week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks. In embodiments, a pharmaceutical composition is administered every month, 2 months, 3 months, 4 months, 5 months, 6 months, 7 months, 8 months, 9 months, 10 months, 11 months, or 12 months.

In embodiments, provided is a method of treating systemic lupus erythematosus comprising administering an effective amount of a composition provided herein wherein the administering is done every 4 weeks. In embodiments, provided is a method of treating systemic lupus erythematosus comprising administering an effective amount of a composition provided herein wherein the administering is done every 12 weeks.

In accordance with a method of the disclosure, an anti-ILT7 binding protein as defined elsewhere herein is used to promote a positive therapeutic response with respect to an autoimmune response. By “positive therapeutic response” with respect to an autoimmune treatment is intended an improvement in a disease in association with the activity of an anti-ILT7 binding protein. Exemplary positive therapeutic responses include but are not limited to a decrease in interferon-alpha levels, a decrease in levels or activity of plasmacytoid dendritic cells, or a decrease in one or more symptoms associated with the disease can be observed. In embodiments, an improvement in a disease can be characterized as a complete response. By “complete response” is intended an absence of clinically detectable disease with normalization of any previously test results. In embodiments, such a response may persist for at least one month following administration according to a method of the disclosure. In embodiments, an improvement in a disease can be categorized as being a partial response. In addition to these positive therapeutic responses, a subject undergoing therapy with the anti-ILT7 binding protein, can experience the beneficial effect of an improvement in the symptoms associated with the disease.

In embodiments, a method disclosed herein can comprise an anti-ILT7 binding protein as part of a method to treat autoimmune diseases and deficiencies or disorders of the immune system that are associated with ILT7 expressing cells. Autoimmune diseases are characterized by cellular, tissue and/or organ injury caused by an immunologic reaction of the subject to its own cells, tissues and/or organs. In embodiments, an autoimmune disease is systemic lupus erythematosus.

Clinical response can be assessed using screening techniques such as magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like.

In embodiments, provided herein is a method for utilizing an anti-ILT7 binding protein for diagnostic monitoring of protein levels in tissue as part of a clinical testing procedure, e.g., to determine efficacy of a given treatment regimen. For example, detection can be facilitated by coupling an anti-ILT7 binding protein to a detectable substance. Examples of detectable substances include various enzymes, prosthetic groups, fluorescent materials, luminescent materials, bioluminescent materials, and radioactive materials. Exemplary enzymes include horseradish peroxidase, alkaline phosphatase, β-galactosidase, or acetylcholinesterase; exemplary prosthetic group complexes include streptavidin/biotin and avidin/biotin; exemplary fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and exemplary radioactive material include ¹²⁵I, ¹³¹I, ³⁵S, or ³H.

In embodiments, disclosed herein are in vitro test methods for selecting a subject for treatment of disease, comprising determining if the subject has an autoimmune disease provided herein. In embodiments, an in vitro method may be performed on biological samples (e.g., blood, cartilage, bone, serum, etc.) extracted from a subject and may be used in determining a mode or course of treatment for a particular subject, e.g., whether to administer an anti-ILT7 binding protein to the subject (alone or in combination with another compound).

In embodiments, disclosed herein are methods to administer to a subject an anti-ILT7 binding protein. In embodiments, disclosed herein are methods to administer to a subject in need thereof an anti-ILT7 binding protein. In embodiments, disclosed herein are methods to administer to a subject in need thereof an anti-ILT7 binding protein, wherein the subject has an autoimmune disease or disorder diagnosis. In embodiments, disclosed herein are methods to administer to a subject in need thereof an anti-ILT7 binding protein, wherein the subject has symptoms of an autoimmune disease or disorder.

In embodiments, disclosed herein are methods to administer to a subject a composition comprising an anti-ILT7 binding protein. In embodiments, disclosed herein are methods to administer to a subject in need thereof a composition comprising an anti-ILT7 binding protein. In embodiments, disclosed herein are methods to administer to a subject in need thereof a composition comprising an anti-ILT7 binding protein, wherein the subject has an autoimmune disease or disorder diagnosis. In embodiments, disclosed herein are methods to administer to a subject in need thereof a composition comprising an anti-ILT7 binding protein, wherein the subject has symptoms of an autoimmune disease or disorder.

In embodiments, disclosed herein are methods to administer to a subject a formulation comprising an anti-ILT7 binding protein. In embodiments, disclosed herein are methods to administer to a subject in need thereof a formulation an anti-ILT7 binding protein. In embodiments, disclosed herein are methods to administer to a subject in need thereof a formulation comprising an anti-ILT7 binding protein, wherein the subject has an autoimmune disease or disorder diagnosis. In embodiments, disclosed herein are methods to administer to a subject in need thereof a formulation comprising an anti-ILT7 binding protein, wherein the subject has symptoms of an autoimmune disease or disorder.

In embodiments, disclosed herein are methods to administer to a subject a composition comprising an anti-ILT7 binding protein. In embodiments, disclosed herein are methods to administer to a subject in need thereof a composition comprising an anti-ILT7 binding protein. In embodiments, disclosed herein are methods to administer to a subject in need thereof a composition comprising an anti-ILT7 binding protein, wherein the subject has an autoimmune disease or disorder diagnosis. In embodiments, disclosed herein are methods to administer to a subject in need thereof a composition comprising an anti-ILT7 binding protein, wherein the subject has symptoms of an autoimmune disease or disorder.

In embodiments, disclosed herein are methods to administer to a subject a composition comprising an anti-ILT7 binding protein; wherein the composition is stored in a pre-filled syringe prior to administration. In embodiments, disclosed herein are methods to administer to a subject in need thereof a composition comprising an anti-ILT7 binding protein; wherein the composition is stored in a pre-filled syringe prior to administration. In embodiments, disclosed herein are methods to administer to a subject in need thereof a composition comprising an anti-ILT7 binding protein, wherein the subject has an autoimmune disease or disorder diagnosis; wherein the composition is stored in a pre-filled syringe prior to administration. In embodiments, disclosed herein are methods to administer to a subject in need thereof a composition comprising an anti-ILT7 binding protein, wherein the subject has symptoms of an autoimmune disease or disorder; wherein the composition is stored in a pre-filled syringe prior to administration.

In embodiments, disclosed herein are methods to administer to a subject a composition comprising 150 mg/mL of an anti-ILT7 binding protein, 20 mM of histidine/histidine-HCl, 180 mM of sucrose, 15 mM L-methionine, and 0.02% polysorbate-80. In embodiments, disclosed herein are methods to administer to a subject in need thereof a composition comprising 150 mg/mL of an anti-ILT7 binding protein, 20 mM of histidine/histidine-HCl, 180 mM of sucrose, 15 mM L-methionine, and 0.02% polysorbate-80. In embodiments, disclosed herein are methods to administer to a subject in need thereof a composition comprising 150 mg/mL of an anti-ILT7 binding protein, 20 mM of histidine/histidine-HCl, 180 mM of sucrose, 15 mM L-methionine, and 0.02% polysorbate-80, wherein the subject has an autoimmune disease or disorder diagnosis. In embodiments, disclosed herein are methods to administer to a subject in need thereof a composition comprising 150 mg/mL of an anti-ILT7 binding protein, 20 mM of histidine/histidine-HCl, 180 mM of sucrose, 15 mM L-methionine, and 0.02% polysorbate-80, wherein the subject has symptoms of an autoimmune disease or disorder.

In embodiments, disclosed herein are methods to administer to a subject a composition comprising 150 mg/mL of an anti-ILT7 binding protein, 20 mM of histidine/histidine-HCl, 180 mM of sucrose, 15 mM L-methionine, and 0.02% polysorbate-80, wherein the composition is stored in a pre-filled syringe prior to administration. In embodiments, disclosed herein are methods to administer to a subject in need thereof a composition comprising 150 mg/mL of an anti-ILT7 binding protein, 20 mM of histidine/histidine-HCl, 180 mM of sucrose, 15 mM L-methionine, and 0.02% polysorbate-80, wherein the composition is stored in a pre-filled syringe prior to administration. In embodiments, disclosed herein are methods to administer to a subject in need thereof a composition comprising 150 mg/mL of an anti-ILT7 binding protein, 20 mM of histidine/histidine-HCl, 180 mM of sucrose, 15 mM L-methionine, and 0.02% polysorbate-80, wherein the subject has an autoimmune disease or disorder diagnosis; wherein the composition is stored in a pre-filled syringe prior to administration. In embodiments, disclosed herein are methods to administer to a subject in need thereof a composition comprising 150 mg/mL of an anti-ILT7 binding protein, 20 mM of histidine/histidine-HCl, 180 mM of sucrose, 15 mM L-methionine, and 0.02% polysorbate-80, wherein the subject has symptoms of an autoimmune disease or disorder; wherein the composition is stored in a pre-filled syringe prior to administration.

### NUMBERED EMBODIMENTS

Notwithstanding the appended claims, the disclosure sets forth the following numbered embodiments:

### Embodiment Set 1

Embodiment 1. A composition, comprising: (i) about 100 mg/mL to about 350 mg/mL of an immunoglobulin-like transcript 7 (ILT7) binding protein; (ii) about 50 to about 350 mM sucrose; (iii) about 0.001% to about 1% polysorbate-80; (iv) L-methionine; and (v) a buffer selected from a histidine buffer, a citrate buffer, or a phosphate buffer.

Embodiment 2. A composition, comprising: (i) about 100 mg/mL to about 165 mg/mL of an immunoglobulin-like transcript 7 (ILT7) binding protein; (ii) about 150 to about 240 mM sucrose; (iii) about 0.003% to about 0.8% polysorbate-80; (iv) L-methionine; and (v) a buffer selected from a histidine buffer, a citrate buffer, or a phosphate buffer.

Embodiment 3. The composition of any one of embodiments 1-2, wherein the buffer is the histidine buffer.

Embodiment 4. The composition of any one of embodiments 1-2, wherein the buffer is the phosphate buffer.

Embodiment 5. The composition of any one of embodiments 1-2, wherein the buffer is the citrate buffer.

Embodiment 6. The composition of any one of embodiments 1-5, wherein a pH of the composition is acidic as determined by a pH meter.

Embodiment 7. The composition of embodiment 6, wherein the acidic pH is from about 5 to about 7 as determined by pH meter.

Embodiment 8. The composition of embodiment 7, wherein the acidic pH is about 6 as determined by pH meter.

Embodiment 9. The composition of any one of embodiments 1-8, wherein the composition comprises from about 5 mM to about 25 mM histidine/histidine-HCl.

Embodiment 10. The composition of embodiment 9, comprising about 20 mM histidine/histidine-HCl.

Embodiment 11. The composition of any one of embodiments 1-10, wherein the composition comprises about 5 mM to about 50 mM L-methionine.

Embodiment 12. The composition of embodiment 11, comprising about 15 mM L-methionine.

Embodiment 13. The composition of any one of embodiments 1-12, comprising about 0.02% polysorbate-80.

Embodiment 14. The composition of any one of embodiments 1-13, comprising up to about 150 mg/mL of the ILT7 binding protein.

Embodiment 15. The composition of any one of embodiments 1-14, comprising about 150 mg/mL of the ILT7 binding protein.

Embodiment 16. The composition of any one of embodiments 1-15, wherein the ILT7 binding protein comprises: i. a VH comprising at least 95%, 96%, 97%, 98% or 99% identity to SEQ ID NO: 1; ii. a VL comprising at least 95%, 96%, 97%, 98% or 99% identity to SEQ ID NO: 2; or iii. i. and ii.

Embodiment 17. The composition of any one of embodiments 1-16, wherein the ILT7 binding protein comprises: i. a VH comprising SEQ ID NO: 1; ii. a VL comprising SEQ ID NO: 2; or iii. i. and ii.

Embodiment 18. The composition of any one of embodiments 1-17, wherein the ILT7 binding protein comprises Complementarity-Determining Regions (CDRs) HCDR1, HDR2, HCDR3, LCDR1, LCDR2, and LCDR3 comprising the sequences of SEQ ID NOs: 3, 4, 5, 6, 7, and 8, respectively.

Embodiment 19. The composition of any one of embodiment 1-18, wherein the composition comprises a viscosity of about 5 cP to about 50 cP as determined by a viscometer.

Embodiment 20. The composition of any one of embodiments 1-19, wherein the ILT7 binding protein is daxdilimab.

Embodiment 21. A composition, comprising about: i. 150 mg/mL of an immunoglobulin-like transcript 7 (ILT7) binding protein; ii. 20 mM histidine/histidine-HCl; iii. 180 mM Sucrose; iv. 15 mM L-Methionine; and v. 0.02% Polysorbate-80.

Embodiment 22. A composition, comprising about: i. 150 mg/ml of an immunoglobulin-like transcript 7 (ILT7) binding protein; ii. 20 mM histidine/histidine-HCl; iii. 180 mM Sucrose; iv. 15 mM L-Methionine; and v. 0.02% Polysorbate-80, wherein the ILT7 binding protein comprises: a VH comprising SEQ ID NO: 1 and a VL comprising SEQ ID NO: 2.

Embodiment 23. A composition, comprising about: i. 150 mg/ml of an immunoglobulin-like transcript 7 (ILT7) binding protein; ii. 20 mM histidine/histidine-HCl; iii. 180 mM Sucrose; iv. 15 mM L-Methionine; and v. 0.02% Polysorbate-80, wherein the ILT7 binding protein comprises Complementarity-Determining Regions (CDRs) HCDR1, HDR2, HCDR3, LCDR1, LCDR2, and LCDR3 comprising the sequences of SEQ ID NOs: 3, 4, 5, 8, 7, and 8, respectively.

Embodiment 24. The composition of any one of embodiments 21-23, wherein viscosity of the composition is from about 5 to about 50 cP.

Embodiment 25. The composition of embodiment 24, wherein the viscosity does not exceed 20 cP.

Embodiment 26. The composition of any one of embodiments 21-25, wherein the rate of aggregation is reduced as compared to an otherwise comparable composition lacking the L-Methionine as determined by size exclusion chromatography at 40° C.

Embodiment 27. The composition of any one of embodiments 21-26, wherein the rate of aggregation is from 0.5% to 1% per month as determined by size exclusion chromatography at 40° C.

Embodiment 28. The composition of any one of embodiments 21-27, wherein a level of oxidation is reduced as compared to an otherwise comparable composition lacking the L-Methionine as determined by peptide mapping of W102 or W104 of SEQ ID NO: 1 or SEQ ID NO: 10 of the ILT7 binding protein at room temperature.

Embodiment 29. The composition of any one of embodiments 21-28, wherein when the composition is exposed to light at 1600-1800 lux for 5 days, the percentage increase of oxidized species of ILT7 binding protein in the composition is at most 0.5% as determined by size exclusion chromatography.

Embodiment 30. The composition of any one of embodiments 21-29, wherein the composition is Fc oxidation resistant.

Embodiment 31. A container comprising the composition of any one of embodiments 1-30 in unit dose form.

Embodiment 32. The container of embodiment 31, wherein the container comprises nitrogen.

Embodiment 33. The container of embodiment 31, wherein the container does not comprise nitrogen.

Embodiment 34. A method of treating a disease comprising administering an effective amount of the composition of any one of embodiments 1-30 to a subject in need thereof, thereby treating the disease.

Embodiment 35. The method of embodiment 34, wherein the administering is sufficient to reduce a symptom of the disease in the subject in need thereof after the administering.

Embodiment 36. The method of embodiment 35, wherein the symptom is reduced by at least about 1-fold.

Embodiment 37. A method of treating an autoimmune disease comprising administering an effective amount of the composition of any of embodiments 1-30 to a subject in need thereof, thereby treating the autoimmune disease.

Embodiment 38. The method of embodiment 37, wherein the autoimmune disease is selected from the group consisting of: Discoid lupus erythematosus (DLE), Focal segmental glomerulosclerosis (FSGS), alopecia areata, myositis, diabetes, Hashimoto's disease, autoimmune adrenal insufficiency, pure red cell anemia, lupus nephritis, multiple sclerosis, IgG4RD, rheumatoid carditis, systemic lupus erythematosus, psoriasis, rheumatoid arthritis, chronic inflammation, Sjogren's syndrome, polymyositis, dermatomyositis, inclusion body myositis, juvenile myositis, and scleroderma.

Embodiment 39. The method of embodiment 38, wherein the autoimmune disease is alopecia areata.

Embodiment 40. The method of embodiment 38, wherein the autoimmune disease is systemic lupus erythematosus.

Embodiment 41. The method of embodiment 38, wherein the autoimmune disease is FSGS.

Embodiment 42. A method of treating alopecia areata comprising administering an effective amount of the composition of any of embodiments 1-30 to a subject in need thereof, thereby treating the alopecia areata.

Embodiment 43. A method of treating Focal segmental glomerulosclerosis (FSGS) comprising administering an effective amount of the composition of any of embodiments 1-30 to a subject in need thereof, thereby treating the FSGS.

Embodiment 44. A method of treating systemic lupus erythematosus comprising administering an effective amount of the composition of any of embodiments 1-30 to a subject in need thereof, thereby treating the systemic lupus erythematosus.

Embodiment 45. The method of embodiment 44, wherein the administering is every 4 weeks.

Embodiment 46. The method of embodiment 44, wherein the administering is every 12 weeks.

Embodiment 47. A method of treating myositis comprising administering an effective amount of the composition of any of embodiments 1-30 to a subject in need thereof, thereby treating the myositis.

Embodiment 48. The method of any one of embodiments 37-47, wherein the administering is subcutaneous.

Embodiment 49. A composition, comprising about: i. 150 mg/ml of an immunoglobulin-like transcript 7 (ILT7) binding protein; ii. 20 mM histidine/histidine-HCl; iii. 180 mM Sucrose; iv. 15 mM L-Methionine; and v. 0.02% Polysorbate-80, wherein the ILT7 binding protein comprises a sequence having at least 85% identity to a sequence of Table 1.

Embodiment 50. A composition, comprising about: i. 150 mg/ml of an immunoglobulin-like transcript 7 (ILT7) binding protein; ii. 20 mM histidine/histidine-HCl; iii. 180 mM Sucrose; iv. 15 mM L-Methionine; and v. 0.02% Polysorbate-80, wherein the ILT7 binding protein comprises a VH comprising SEQ ID NO:1, a VL comprising SEQ ID NO: 2, or both the VH and VL of SEQ ID NO: 1 and SEQ ID NO: 2, respectively.

Embodiment 51. A method of treating an autoimmune disease comprising administering a composition that comprises: i. about 150 mg/mL of an immunoglobulin-like transcript 7 (ILT7) binding protein; ii. 20 mM histidine/histidine-HCl; iii. 180 mM Sucrose; iv. 15 mM L-Methionine; and v. 0.02% Polysorbate-80, wherein the ILT7 binding protein comprises Complementarity-Determining Regions (CDRs) HCDR1, HDR2, HCDR3, LCDR1, LCDR2, and LCDR3 comprising the sequences of SEQ ID NOs: 3, 4, 5, 6, 7, and 8 respectively, wherein the dose of the ILT7 binding protein comprises from about 150 mg to about 350 mg; wherein the administration is subcutaneous, and wherein the dose is administered in a single injection.

Embodiment 52. The method of embodiment 51, wherein the dose of the ILT7 binding protein comprises from about 150 mg to about 275 mg.

Embodiment 53. The method of embodiment 52, wherein the dose of the ILT7 binding protein comprises from about 150 mg to about 200 mg.

### Embodiment Set 2

Embodiment 54. A composition, comprising: (i) about 100 mg/mL to about 350 mg/mL of an immunoglobulin-like transcript 7 (ILT7) binding protein; (ii) about 50 to about 350 mM sucrose; (iii) about 0.001% to about 1% polysorbate-80; (iv) L-methionine; and (v) a buffer selected from a histidine buffer, a citrate buffer, or a phosphate buffer.

Embodiment 55. A composition, comprising: (i) about 100 mg/mL to about 165 mg/mL of an immunoglobulin-like transcript 7 (ILT7) binding protein; (ii) about 150 to about 240 mM sucrose; (iii) about 0.003% to about 0.8% polysorbate-80; (iv) L-methionine; and (v) a buffer selected from a histidine buffer, a citrate buffer, or a phosphate buffer.

Embodiment 56. The composition of any one of embodiments 54-55, wherein the buffer is the histidine buffer.

Embodiment 57. The composition of any one of embodiments 54-55, wherein the buffer is the phosphate buffer.

Embodiment 58. The composition of any one of embodiments 54-55, wherein the buffer is the citrate buffer.

Embodiment 59. The composition of any one of embodiments 54-58, wherein a pH of the composition is acidic as determined by a pH meter.

Embodiment 60. The composition of embodiment 59, wherein the acidic pH is from about 5 to about 7 as determined by pH meter.

Embodiment 61. The composition of embodiment 60, wherein the acidic pH is about 6 as determined by pH meter.

Embodiment 62. The composition of any one of embodiments 54-62, wherein the composition comprises from about 5 mM to about 25 mM histidine/histidine-HCl.

Embodiment 63. The composition of embodiment 62, comprising about 20 mM histidine/histidine-HCl.

Embodiment 64. The composition of any one of embodiments 54-63, wherein the composition comprises about 5 mM to about 50 mM L-methionine.

Embodiment 65. The composition of embodiment 64, comprising about 15 mM L-methionine.

Embodiment 66. The composition of any one of embodiments 54-65, comprising about 0.02% polysorbate-80.

Embodiment 67. The composition of any one of embodiments 54-66, comprising up to about 150 mg/mL of the ILT7 binding protein.

Embodiment 68. The composition of any one of embodiments 54-67, comprising about 150 mg/mL of the ILT7 binding protein.

Embodiment 69. The composition of any one of embodiments 54-68, wherein the ILT7 binding protein comprises: i. a VH comprising at least 95%, 96%, 97%, 98% or 99% identity to SEQ ID NO: 1; ii. a VL comprising at least 95%, 96%, 97%, 98% or 99% identity to SEQ ID NO: 2; or iii. i. and ii.

Embodiment 70. The composition of any one of embodiments 54-69, wherein the ILT7 binding protein comprises: i. a VH comprising SEQ ID NO: 1; ii. a VL comprising SEQ ID NO: 2; or iii. i. and ii.

Embodiment 71. The composition of any one of embodiments 54-70, wherein the ILT7 binding protein comprises Complementarity-Determining Regions (CDRs) HCDR1, HDR2, HCDR3, LCDR1, LCDR2, and LCDR3 comprising the sequences of SEQ ID NOs: 3, 4, 5, 6, 7, and 8, respectively.

Embodiment 72. The composition of any one of embodiment 54-71, wherein the composition comprises a viscosity of about 5 cP to about 50 cP as determined by a viscometer.

Embodiment 73. The composition of any one of embodiments 54-72, wherein the ILT7 binding protein is daxdilimab.

Embodiment 74. A composition, comprising about: i. 150 mg/mL of an immunoglobulin-like transcript 7 (ILT7) binding protein; ii. 20 mM histidine/histidine-HCl; iii. 180 mM Sucrose; iv. 15 mM L-Methionine; and v. 0.02% Polysorbate-80.

Embodiment 75. A composition, comprising about: i. 150 mg/ml of an immunoglobulin-like transcript 7 (ILT7) binding protein; ii. 20 mM histidine/histidine-HCl; iii. 180 mM Sucrose; iv. 15 mM L-Methionine; and v. 0.02% Polysorbate-80, wherein the ILT7 binding protein comprises: a VH comprising SEQ ID NO: 1 and a VL comprising SEQ ID NO: 2.

Embodiment 76. A composition, comprising about: i. 150 mg/ml of an immunoglobulin-like transcript 7 (ILT7) binding protein; ii. 20 mM histidine/histidine-HCl; iii. 180 mM Sucrose; iv. 15 mM L-Methionine; and v. 0.02% Polysorbate-80, wherein the ILT7 binding protein comprises Complementarity-Determining Regions (CDRs) HCDR1, HDR2, HCDR3, LCDR1, LCDR2, and LCDR3 comprising the sequences of SEQ ID NOs: 3, 4, 5, 8, 7, and 8, respectively.

Embodiment 77. The composition of any one of embodiments 54-76, wherein viscosity of the composition is from about 5 to about 50 cP.

Embodiment 78. The composition of embodiment 77, wherein the viscosity does not exceed 20 cP.

Embodiment 79. The composition of any one of embodiments 54-78, wherein the rate of aggregation is reduced as compared to an otherwise comparable composition lacking the L-Methionine as determined by size exclusion chromatography at 40° C.

Embodiment 80. The composition of any one of embodiments 54-79, wherein the rate of aggregation is from 0.5% to 1% per month as determined by size exclusion chromatography at 40° C.

Embodiment 81. The composition of any one of embodiments 54-80, wherein a level of oxidation is reduced as compared to an otherwise comparable composition lacking the L-Methionine as determined by peptide mapping of W102 or W104 of SEQ ID NO: 1 or SEQ ID NO: 10 of the ILT7 binding protein at room temperature.

Embodiment 82. The composition of any one of embodiments 54-81, wherein when the composition is exposed to light at 1600-1800 lux for 5 days, the percentage increase of oxidized species of ILT7 binding protein in the composition is at most 0.5% as determined by size exclusion chromatography.

Embodiment 83. The composition of any one of embodiments 54-82, wherein the composition is Fc oxidation resistant.

Embodiment 84. A container comprising the composition of any one of embodiments 54-83 in unit dose form.

Embodiment 85. The container of embodiment 84, wherein the container comprises nitrogen.

Embodiment 86. The container of embodiment 84, wherein the container does not comprise nitrogen.

Embodiment 87. A method of treating a disease comprising administering an effective amount of the composition of any one of embodiments 54-83 to a subject in need thereof, thereby treating the disease.

Embodiment 88. The method of embodiment 87, wherein the administering is sufficient to reduce a symptom of the disease in the subject in need thereof after the administering.

Embodiment 89. The method of embodiment 88, wherein the symptom is reduced by at least about 1-fold.

Embodiment 90. A method of treating an autoimmune disease comprising administering an effective amount of the composition of any of embodiments 54-83 to a subject in need thereof, thereby treating the autoimmune disease.

Embodiment 91. The method of embodiment 90, wherein the autoimmune disease is selected from the group consisting of: Discoid lupus erythematosus (DLE), Focal segmental glomerulosclerosis (FSGS), alopecia areata, myositis, diabetes, Hashimoto's disease, autoimmune adrenal insufficiency, pure red cell anemia, lupus nephritis, multiple sclerosis, IgG4RD, rheumatoid carditis, systemic lupus erythematosus, psoriasis, rheumatoid arthritis, chronic inflammation, Sjogren's syndrome, polymyositis, dermatomyositis, inclusion body myositis, juvenile myositis, and scleroderma.

Embodiment 92. The method of embodiment 91, wherein the autoimmune disease is alopecia areata.

Embodiment 93. The method of embodiment 91, wherein the autoimmune disease is systemic lupus erythematosus

Embodiment 94. The method of embodiment 91, wherein the autoimmune disease is FSGS.

Embodiment 95. A method of treating alopecia areata comprising administering an effective amount of the composition of any of embodiments 54-83 to a subject in need thereof, thereby treating the alopecia areata.

Embodiment 96. A method of treating Focal segmental glomerulosclerosis (FSGS) comprising administering an effective amount of the composition of any of embodiments 1-98 to a subject in need thereof, thereby treating the FSGS.

Embodiment 97. A method of treating systemic lupus erythematosus comprising administering an effective amount of the composition of any of embodiments 54-83 to a subject in need thereof, thereby treating the systemic lupus erythematosus.

Embodiment 98. The method of embodiment 97, wherein the administering is every 4 weeks.

Embodiment 99. The method of embodiment 97, wherein the administering is every 12 weeks.

Embodiment 100. A method of treating myositis comprising administering an effective amount of the composition of any of embodiments 54-83 to a subject in need thereof, thereby treating the myositis.

Embodiment 101. The method of any one of embodiments 90-100, wherein the administering is subcutaneous.

Embodiment 102. A composition, comprising about: i. 150 mg/ml of an immunoglobulin-like transcript 7 (ILT7) binding protein; ii. 20 mM histidine/histidine-HCl; iii. 180 mM Sucrose; iv. 15 mM L-Methionine; and v. 0.02% Polysorbate-80, wherein the ILT7 binding protein comprises a sequence having at least 85% identity to a sequence of Table 1.

Embodiment 103. A composition, comprising about: i. 150 mg/ml of an immunoglobulin-like transcript 7 (ILT7) binding protein; ii. 20 mM histidine/histidine-HCl; iii. 180 mM Sucrose; iv. 15 mM L-Methionine; and v. 0.02% Polysorbate-80, wherein the ILT7 binding protein comprises a VH comprising SEQ ID NO:1, a VL comprising SEQ ID NO: 2, or both the VH and VL of SEQ ID NO: 1 and SEQ ID NO: 2, respectively.

Embodiment 104. A method of treating an autoimmune disease comprising administering a composition that comprises: i. about 150 mg/mL of an immunoglobulin-like transcript 7 (ILT7) binding protein; ii. 20 mM histidine/histidine-HCl; iii. 180 mM Sucrose; iv. 15 mM L-Methionine; and v. 0.02% Polysorbate-80, wherein the ILT7 binding protein comprises Complementarity-Determining Regions (CDRs) HCDR1, HDR2, HCDR3, LCDR1, LCDR2, and LCDR3 comprising the sequences of SEQ ID NOs: 3, 4, 5, 6, 7, and 8 respectively, wherein the dose of the ILT7 binding protein comprises from about 150 mg to about 350 mg; wherein the administration is subcutaneous, and wherein the dose is administered in a single injection.

Embodiment 105. The method of embodiment 104, wherein the dose of the ILT7 binding protein comprises from about 150 mg to about 275 mg.

Embodiment 106. The method of embodiment 105, wherein the dose of the ILT7 binding protein comprises from about 150 mg to about 200 mg.

Embodiment 107. A delivery device comprising the composition of any one of embodiments 54-83, or 102-103.

Embodiment 108. The delivery device of embodiment 107, wherein the device is an autoinjector.

Embodiment 109. A kit comprising the delivery device of any one or embodiments 107-108, and instructions for use thereof.

Embodiment 110. The kit of embodiment 109, comprising a plurality of autoinjectors.

Embodiment 111. A method of treatment, comprising administering daxdilimab to a subject in need thereof, wherein the administering comprises use of an autoinjector.

Embodiment 112. The method of treatment of embodiment 111, wherein the autoinjector is contacted with the thigh, abdomen, or arm of the subject in a manner sufficient to effectuate the administration of the daxdilimab to the subject in need.

Embodiment 113. The method of any one of embodiments 111-112, wherein the treatment is of an autoimmune disease or condition selected from the group consisting of: Discoid lupus erythematosus (DLE), Focal segmental glomerulosclerosis (FSGS), alopecia areata, lupus nephritis, myositis, diabetes, Hashimoto's disease, autoimmune adrenal insufficiency, pure red cell anemia, multiple sclerosis, rheumatoid carditis, systemic lupus erythematosus, psoriasis, rheumatoid arthritis, chronic inflammation, Sjogren's syndrome, polymyositis, dermatomyositis, inclusion body myositis, juvenile myositis, IgG4 related disease (IgG4RD), and scleroderma.

Embodiment 114. The method of any one of embodiments 111-112, wherein the treatment is of a non-autoimmune disease or condition.

Embodiment 115. The method of embodiment 114, wherein the non-autoimmune disease or condition comprises an ILT7-expressing cell-mediated disease.

Embodiment 116. A composition, comprising: (i) an immunoglobulin-like transcript 7 (ILT7) binding protein; and (ii) L-methionine; wherein when the composition is exposed to light at 1600-1800 lux for 5 days, the increase in percentage of oxidized species of ILT7 binding protein in the composition is at most about 1.0% as determined by size exclusion chromatography.

Embodiment 117. The composition of embodiment 116, wherein the increase in percentage of oxidized species of ILT7 binding protein in the composition is at most about 0.4%, 0.3%, 0.2%, or 0.1%.

Embodiment 118. A composition, comprising: (i) an immunoglobulin-like transcript 7 (ILT7) binding protein; and (ii) L-methionine; wherein when the composition is exposed to light at 1600-1800 lux the rate of formation of new oxidized species per day of ILT7 binding protein in the composition is at most about 0.1%-0.5% as determined by size exclusion chromatography.

Embodiment 119. The composition of embodiment 118, wherein the rate of formation of new oxidized species per day of the ILT7 binding protein in the composition is at most about 0.2%-0.4% or 0.1%-0.3%.

Embodiment 120. The composition of any one of embodiments 116-119, comprising about 100 mg/mL to about 450 mg/mL of the ILT7 binding protein.

Embodiment 121. The composition of any one of embodiments 116-119, wherein the ILT7 binding protein comprises Complementarity-Determining Regions (CDRs) HCDR1, HDR2, HCDR3, LCDR1, LCDR2, and LCDR3 comprising the sequences of SEQ ID NOs: 3, 4, 5, 6, 7, and 8, respectively.

These and other embodiments are disclosed herein.

### Examples

Light-induced tryptophan oxidation in complementarity-determining regions (CDRs) of some antibody-based therapeutics can impact potency and shelf life. Further, there is a need to eliminate and/or reduce the need for engineering controls that include but are not limited to lighting modulation and the need for a nitrogen overlay during manufacturing.

To evaluate and improve upon existing formulations, an anti-ILT7 binding protein was utilized to evaluate means to reduce and/or eliminate light-induced tryptophan oxidation of CDRs that can impact potency and shelf-life of antibodies, the need for engineering controls, use of a nitrogen overlay during manufacturing, and the use of syringes as storage containers (e.g., pre-filled syringe).

Additionally, formulations that contain higher concentrations of anti-ILT7 binding protein allow for administration of higher doses. Advances regarding higher concentrations have been previously limited due to increases in aggregation rate and formulation viscosity.

### Example 1. Formulation and Evaluation of Rate of Oxidized Species

### Formulation

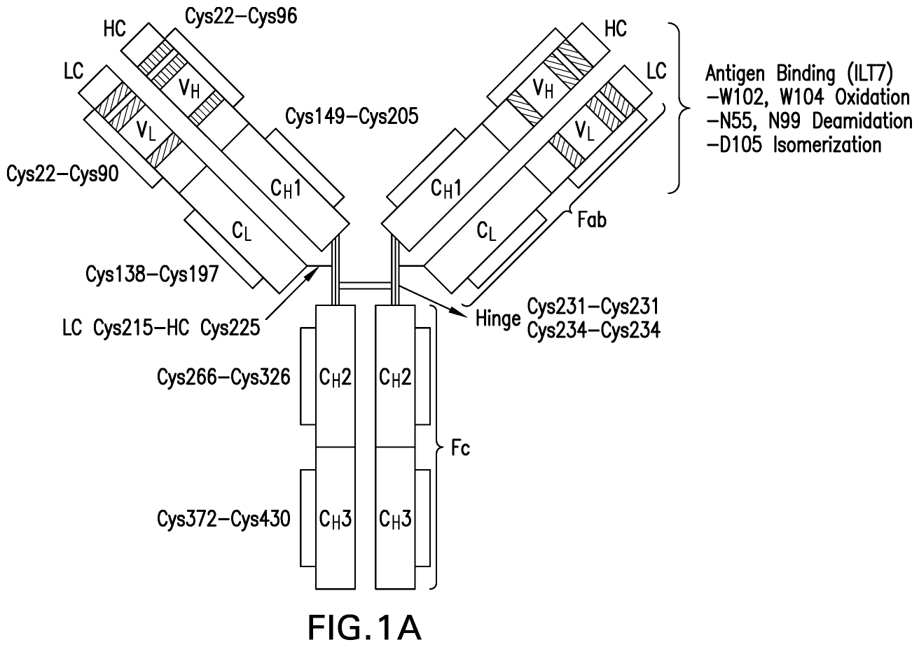

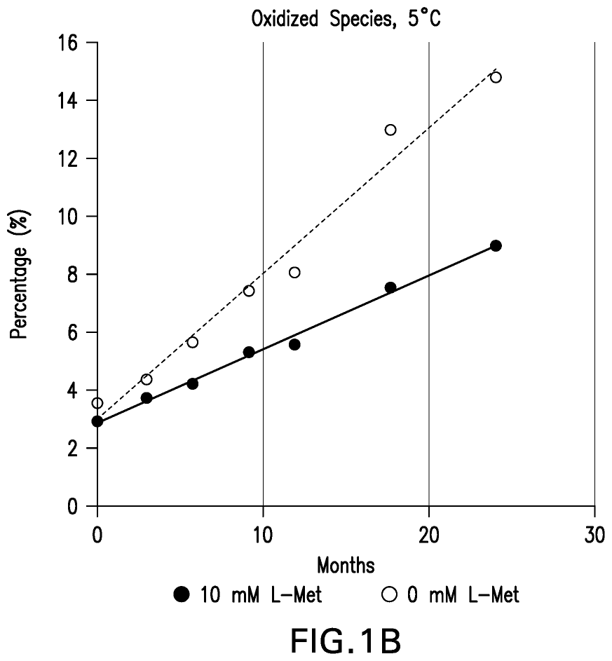

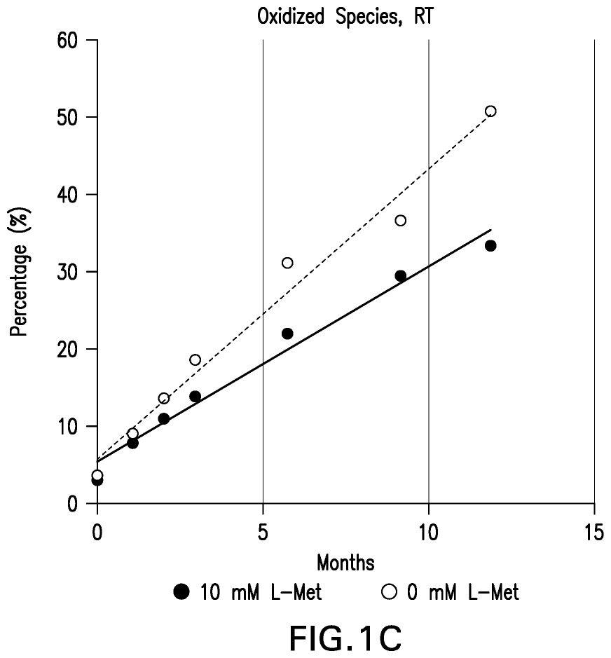

Several problems were identified with existing formulations containing an afucosylated human IgG12 anti-ILT7 mAb; Specifically, light-induced tryptophan oxidation, oxidation on the Fc portion of the antibody, deamidation and isomerization (FIGS. 1A-1C). Previously, a nitrogen overlay has been used to reduce oxidation in a liquid formulation. However, nitrogen overlays add additional manufacturing complexity for liquid formulations. Formulations were modified herein to reduce or eliminate these problems. The disclosed compositions and methods show that the identified problems are resolved with the provided compositions and methods.

### Evaluation of Oxidized Species Formation and Aggregation Rate of L-Methionine on an Anti-ILT7 Monoclonal Antibody

In order to reduce or eliminate the need for engineering controls during manufacturing (e.g., light exposure, nitrogen overlay, and the like), a screening for an improved formulation comprising an anti-ILT7 monoclonal antibody (mAb) and use of excipients was conducted and evaluated for the presence of formation of oxidized species.

In a formulation of 100 mg/mL anti-ILT7 mAb comprising a sequence of Table 1 (SEQ ID NOs: 10 and 11), 20 mM histidine/histidine-HCl, 240 mM sucrose, and 0.02% PS-80 at pH 6.0, the effect of excipient [10 mM L-methionine (L-Met)] versus controls (0 mM L-Met) on oxidation and aggregation rates was assessed. Aggregates were analyzed using size-exclusion chromatography (SEC). Specifically, the effect of excipient (10 mM L-Met) versus control (0 mM L-Met) on oxidized species was assessed at 5° C. and RT and aggregation was assessed at 5° C., RT, and 40° C.

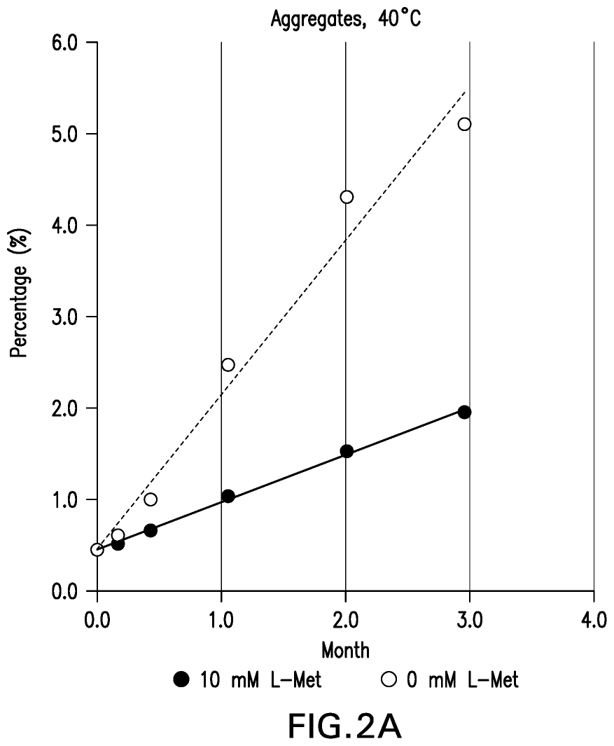

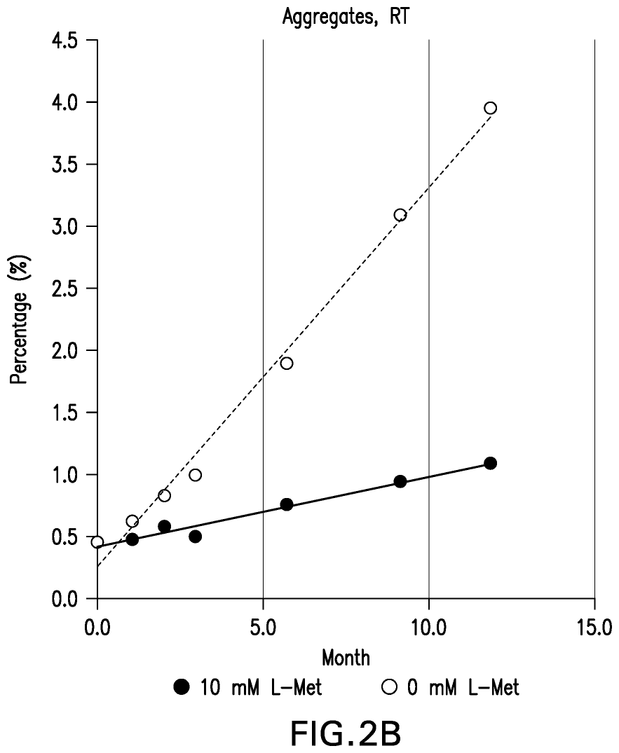

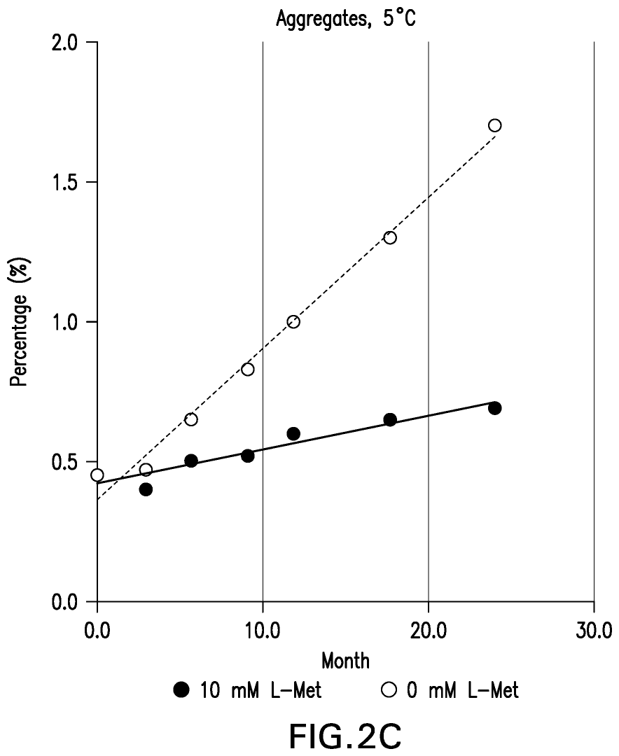

Results show that at 5° C. and RT, L-Met reduced oxidized species rate (FIGS. 1B and 1C respectively). Additionally, at all temperatures (40° C., RT, and 5° C.), L-Met reduced aggregation rate on anti-ILT7 mAb (FIGS. 2A, 2B, and 2C, respectively). Indeed, utilizing the provided composition reduced Met oxidation in the ILT7 mAb composition, which is known to have adverse effects on proteins, including decreased stability and decreased biological activity. Further, the observed reduced aggregation rate supports the use of L-Met in compositions to allow for increased antibody concentration in dosage forms.

### Example 2: Evaluation of L-Methionine in an anti-ILT7 mAb Formulation Under Stressed Conditions

To evaluate whether L-methionine reduces or eliminates oxidation, isomerization, and/or deamidation that can impact antibody potency and shelf-life, formulations were evaluated under stressed conditions that mimic long term storage.

In formulations containing 100 mg/mL anti-ILT7 mAb, 20 mM histidine, 240 mM sucrose, and 0.02% PS-80 at pH 6.0, the effect of 10 mM L-Met or control (0 mM L-Met) on CDR modifications, and Fc oxidation, was assessed at 2 weeks and 1 month under stressed conditions (40° C.) and at 3 months under ambient (RT) conditions. The effects on oxidation, isomerization, and deamidation levels are reported in Table 2.

The data show that including 10 mM L-Met decreased oxidation of the antibody for exemplary residues M257 and M433 of SEQ ID NO: 10. Notably, CDR modification of exemplary residues W102/W104 of SEQ ID NO: 1 or SEQ ID NO: 10 was reduced by about 50% for stressed conditions (40° C.). Oxidation of the Fc residues was also substantially reduced. These surprising results support the utility of the disclosed compositions and methods, particularly in providing for improved antibody compositions with increased stability and ability to store long-term.

TABLE 2
Effects of L-Met on Post-translational Modifications
(PTM) at 40° C. and room temperature (RT)

| | | | 10 mM | 0 mM | 10 mM | 0 mM | 10 mM | 0 mM |
|---|---|---|---|---|---|---|---|---|
| | | | L-Met | L-Met | L-Met | L-Met | L-Met | L-Met |
| | | | 2 W | 2 W | 1 M | 1 M | 3 M | 3 M |
| | | T = 0 | 40° C. | 40° C. | 40° C. | 40° C. | RT | RT |
| Attribute (PTM) | | (%) | (%) | (%) | (%) | (%) | (%) | (%) |
| FC | M257 | 0.7 | 1.2 | 2.5 | 1.2 | 6.4 | 1.4 | 3.7 |
| Oxidation | M433 | 0.5 | 0.7 | 1.1 | 0.8 | 2.1 | 0.4 | 0.9 |
| CDR | W102/W104 | 0.0 | 0.2 | 0.4 | 0.5 | 1.2 | 1.4 | 2.4 |
| Modification | | | | | | | | |

T0 = starting timepoint;
2 W = 2 weeks;
1 M = 1 month;
3 M = 3 months

### Example 3. Evaluation of Headspace of an Anti-ILT7 mAb Formulation

In formulations containing 100 mg/mL anti-ILT7 mAb, 20 mM histidine, 240 mM sucrose, and 0.02% PS-80 at pH 6.0, the effect of headspace (nitrogen and air) in formulations without L-Met on CDR modifications and Fc oxidation of specific species were assessed.

Results in Example 2 showed that the presence of 10 mM L-Met produced a lower percentage of oxidation at RT as compared to conditions lacking L-Met (0 mM) for exemplary residues M257 and M433 of SEQ ID NO: 10. In comparison, the addition of a nitrogen overlay in conditions lacking L-Met (0 mM) produced a lower percentage of oxidation as compared to air for the same exemplary residues M257 and M433 of SEQ ID NO: 10.

Results in Example 2 (Table 2) also showed that the presence of 10 mM L-Met produced a lower percentage of CDR modification at RT as compared to conditions lacking L-Met (0 mM) for the exemplary residues W102/W104 (SEQ ID NOs: 1 and 10). In comparison, the addition of a nitrogen overlay in conditions lacking L-Met (0 mM) produced a lower percentage of oxidized species as compared to air for the same exemplary residues W102/W104 (SEQ ID NOs: 1 and 10). CDR modifications (W102/W104 of SEQ ID NO: 1 or SEQ ID NO: 10 and M257/M433 of SEQ ID NO: 10) yielded a similar, lower percentage of oxidized species in the 10 mM L-Met (1.4%; Table 2) and nitrogen overlay (1.1%; Table 3) groups. These data show that the presence of L-Met in formulations enhances their stability and can obviate the need for nitrogen overlays thereby reducing labor and costs of generating these formulations as compared to existing methods.

Results reporting the effects described above of headspace on PTMs are provided in Table 3.

TABLE 3
Effect of Headspace on Exemplary Post-Translational Modifications
(PTMs) at 3 Months at Room Temperature (RT)

| | | | P2 | P2 |
|---|---|---|---|---|
| | | | Air | Nitrogen |
| | | T0 | 3 M RT | 3 M RT |
| Attribute (PTM) | | (%) | (%) | (%) |
| FC | M257 | 1.0 | 3.8 | 1.5 |
| Oxidation | M433 | 0.3 | 0.9 | 0.5 |
| CDR | W102/ | 0.5 | 2.4 | 1.1 |
| Modification | W104 | | | |

P2: 100 mg/mL HZN7734, 20 mM histidine, 240 mM sucrose, 0.02% PS-80 pH 6.0;
T0 = starting timepoint

### Example 4. Evaluation of L-Methionine on Oxidation and Aggregation of a 150 mg/mL Formulation of an anti-ILT7 mAb

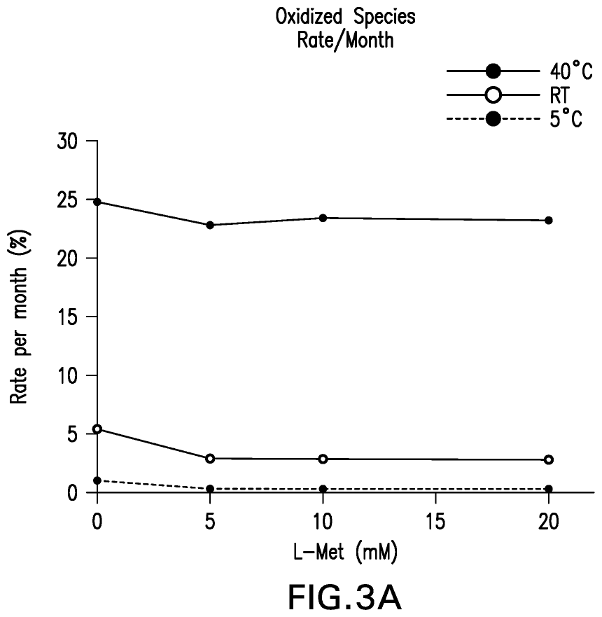

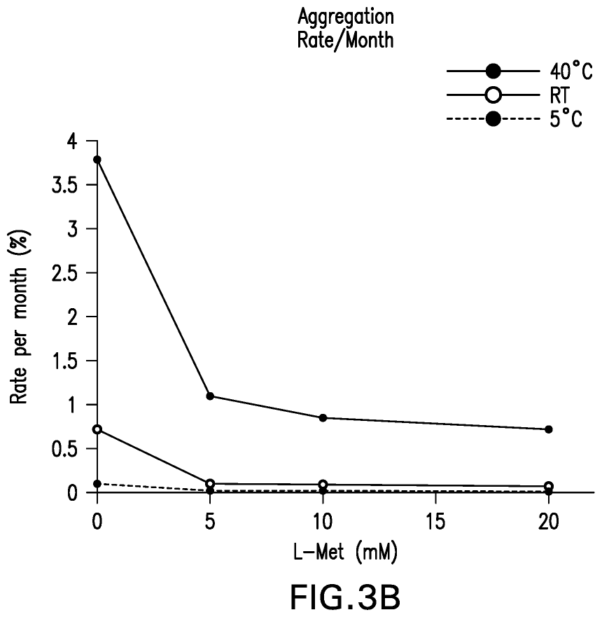

In formulations containing 150 mg/mL anti-ILT7 mAb, 20 mM histidine, 200 mM sucrose, and 0.02% PS-80 at pH 6.0, the effect of L-Met (0 mM, 5 mM, 10 mM, 15 mM, and 20 mM) on oxidation (FIG. 3A) and aggregation (FIG. 3B) rates was assessed at 5° C., RT, and 40° C. Results show that 15 mM L-Met resulted in stabilization and robustness.

Further, the effect of L-Met (0 mM, 10 mM, and 20 mM) on CDR modifications, and Fc oxidation, was assessed at 3 months at RT. Results show that L-Met decreased FC oxidation levels of exemplary residues M257 and M433 (SEQ ID NO: 10) at RT, and L-Met decreased CDR modification at exemplary residues W102/W104 (SEQ ID NOs: 1 and 10), see Table 4. Indeed, the use of L-Methionine provided for reduced oxidation and aggregation, thereby supporting its use to achieve formulations with improved potency, stability, and flexibility in achieving higher dosing amounts in therapeutic regimens.

TABLE 4
Effects of L-Met on PTMs at 3 Months at RT

| | | T0 | 3 M RT | T0 | 3 M RT | T0 | 3 M RT |
|---|---|---|---|---|---|---|---|
| | | 0 mM | 0 mM | 10 mM | 10 mM | 20 mM | 20 mM |
| Attribute (PTM) | | L-Met | L-Met | L-Met | L-Met | L-Met | L-Met |
| FC Oxidation | M257 | 1.2 | 6.7 | 1.2 | 1.5 | 1.1 | 1.4 |
| | M433 | 0.3 | 1.4 | 0.3 | 0.4 | 0.3 | 0.3 |
| CDR | W102/W104 | 0.3 | 3.6 | 0.4 | 1.2 | 0.4 | 1.1 |
| Modification | | | | | | | |

T0 = starting timepoint;
3 M = 3 months.

### Example 5. Evaluation of the Effect of L-Methionine on Oxidation of an anti-ILT7 mAb Formulation under Light and Dark Conditions

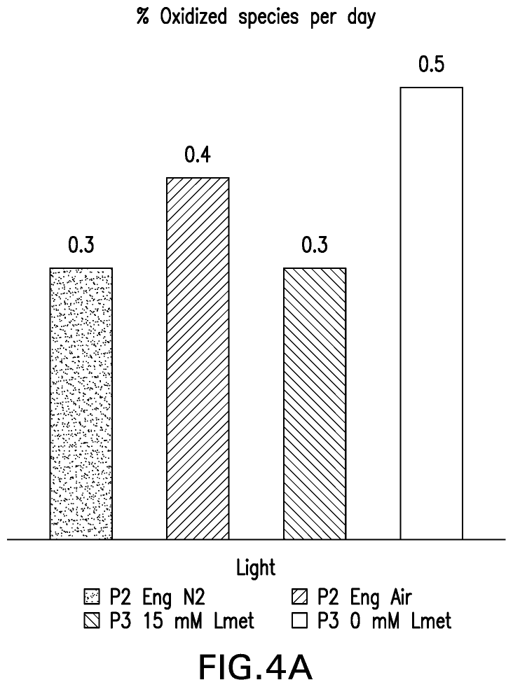

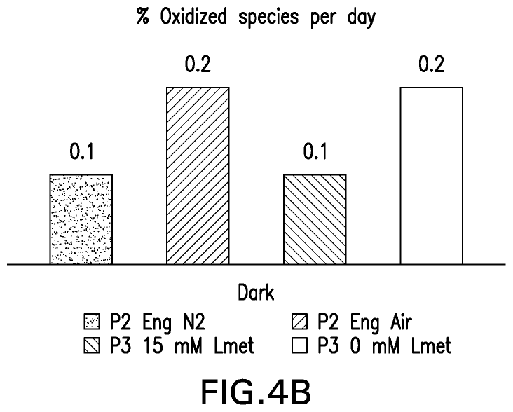

The effect of L-Met on oxidized species formation in formulations exposed to light was assessed. Specifically, the presence of air, nitrogen, or L-Met (0 mM or 15 mM) was assessed. The formulations contained either 100 mg/mL anti-ILT7 mAb, 20 mM histidine/histidine-HCl, 240 mM sucrose, 0.02% PS-80 at pH 6.0 (P2) or 150 mg/mL anti-ILT7 mAb, 20 mM histidine, 200 mM sucrose, and 0.02% PS-80 at pH 6.0 (P3). Vials were held in light box at ambient room temperature and light intensity was measured as 1600-1800 lux throughout the study (FIG. 4A). Vials were held in a dark box at room temperature as a control (FIG. 4B).

It was observed that 15 mM L-Met minimized oxidized species formation when exposed to light for 5 days (FIG. 4A). Results indicate that the use of L-methionine in a formulation is effective in at least reducing light-induced tryptophan oxidation in CDRs of the anti-ILT7 mAb that can impact potency and/or shelf-life. These results provide solutions to existing manufacturing methods that require the need for manufacturing and storage controls to reduce oxidation of antibody formulations.

### Example 6. Evaluation of the Impact of Sucrose Concentration on Oxidation and Aggregation Rates of an Anti-ILT7 Formulation

The impact of sucrose concentration on oxidation and aggregation rates on formulations containing at least 150 mg/mL anti-ILT7 mAb was assessed. Results in Table 5 show that no significant impact of 150-240 mM sucrose was observed.

TABLE 5
Impact of Sucrose Concentration on
Oxidation and Aggregation Rates

| | | | Aggregates | | | Oxidized Species | | |
|---|---|---|---|---|---|---|---|---|
| | Sucrose | L-Met | (%/Month) | | | (%/Month) | | |
| | (mM) | (mM) | 40° C. | RT | 5° C. | 40° C. | RT | 5° C. |
| Exper- | 240 | 0 | 3.8 | 0.7 | 0.10 | 24.6 | 5.4 | 0.8 |
| iment | | | | | | | | |
| I^{a} | 200 | 0 | 3.8 | 0.7 | 0.11 | 24.8 | 5.4 | 0.9 |
| Exper- | 200 | 15 | 1.0 | 0.12 | 0.04 | 23.2 | 3.1 | 0.5 |
| iment | | | | | | | | |
| II^{a} | 150 | 15 | 0.9 | 0.12 | 0.04 | 22.9 | 3.2 | 0.5 |

^{a}Samples held for 3 months at 40° C., 6 months at RT, and 24 months at 5° C.

Additionally, in formulations containing 150 mg/mL anti-ILT7 mAb, 20 mM histidine/histidine-HCl, 15 mM L-Methionine, 0.02% PS-80 at pH 6.0 the effect of decreasing sucrose concentration (150-200 mM) on CDR modifications, and Fc oxidation was assessed at 1 month at 40° C. A minimal effect of decreasing sucrose concentration was observed, see Table 6.

TABLE 6
Impact of Sucrose Concentration on PTMs at 1 Month at 40° C.

| | | T0 | 1 M 40° C. | T0 | 1 M 40° C. |
|---|---|---|---|---|---|
| | | 15 mM | 15 mM | 15 mM | 15 mM |
| | | L-Met | L-Met | L-Met | L-Met |
| | | 200 mM | 200 mM | 150 mM | 150 mM |
| Attribute (PTM) | | Sucrose | Sucrose | Sucrose | Sucrose |
| FC | M257 | 1.1 | 1.7 | 1.1 | 1.9 |
| Oxidation | M433 | 0.4 | 0.5 | 0.3 | 0.5 |
| CDR | W102/ | 0.4 | 1.9 | 0.4 | 2.1 |
| Modifica- | W104 | | | | |
| tion | | | | | |

T0 = starting timepoint;
1 M = 1 month.

### Example 7. Evaluation of the Effect of Anti-ILT7 mAb Concentration on Oxidation Rate

The effect of anti-ILT7 mAb concentration (10 mg/ml, 25 mg/ml, 50 mg/ml, 75 mg/ml, and 100 mg/ml) on formulations containing 20 mM histidine, 240 mM sucrose, and 0.02% PS-80 at pH 6.0 was assessed at 5° C., RT, and 40° C.

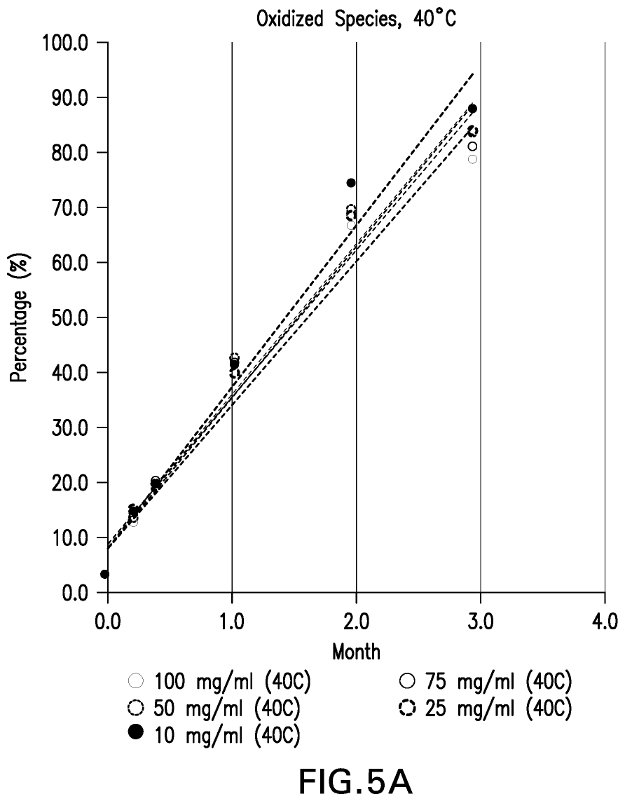

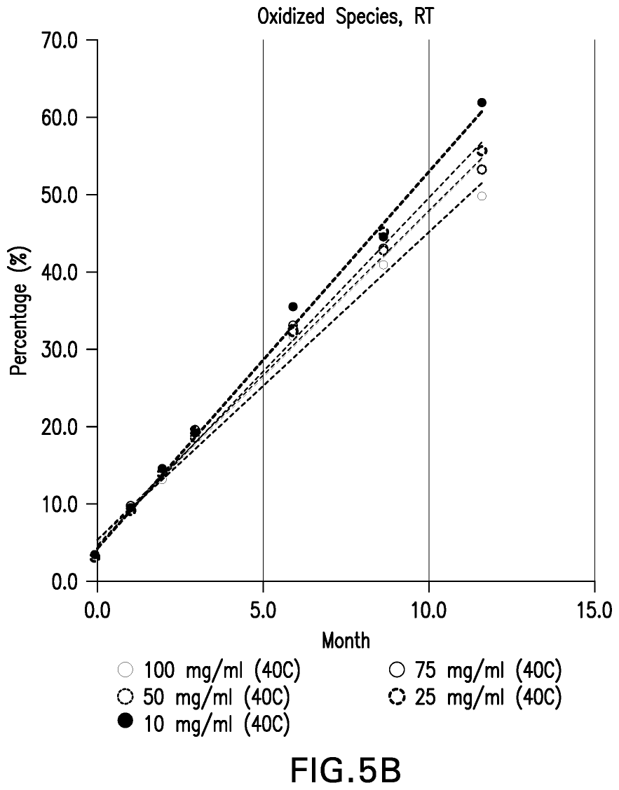

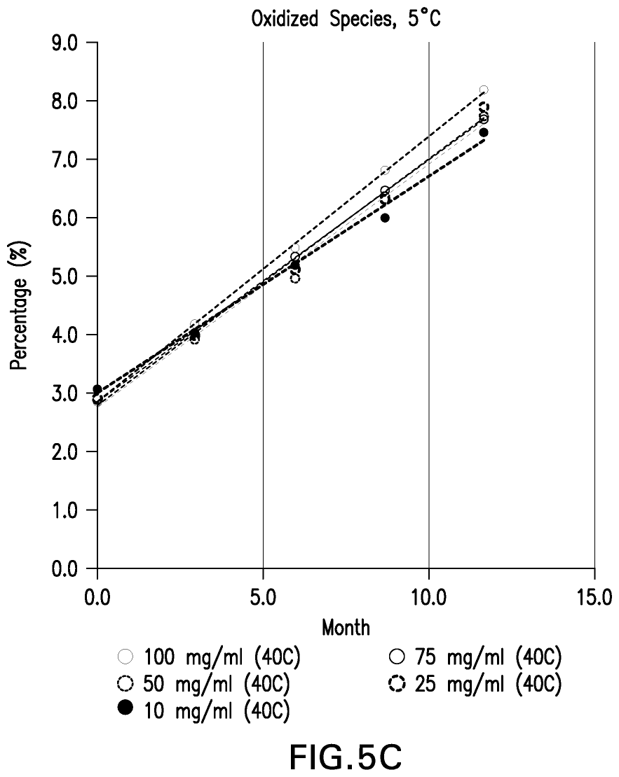

At all temperatures, it was determined that anti-ILT7 mAb concentration did not impact oxidation rate (FIGS. 5A-5C). Therefore, there exists no limitation to the use of varying amounts of mAb concentration in formulations and therapeutic regiments comprising an ILT7 mAb can be adapted accordingly.

### Example 8. Evaluation of the Effect of a Container on Anti-ILT7 mAb Formulation Aggregation and Oxidation

The effect of a storage container (vial or pre-filled syringe (PFS) on aggregation and oxidation of 150 mg/mL anti-ILT7 mAb formulations containing 20 mM histidine, 15 mM methionine, 180 mM sucrose, and 0.02% PS-80 at pH 6.0 was assessed at 5° C. (up to 20 months), 25° C. (up to 6 months), and 40° C. (up to 3 months). The volume of formulation (1 mL and 2 mL) were also tested in the PFS.

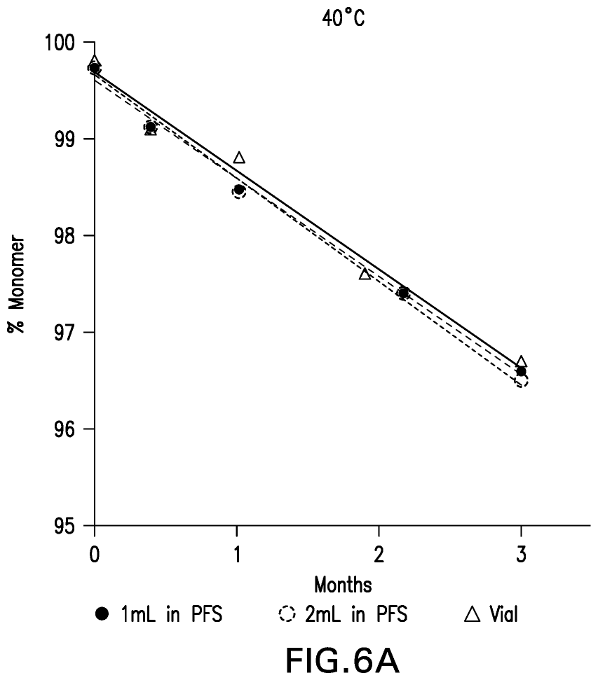

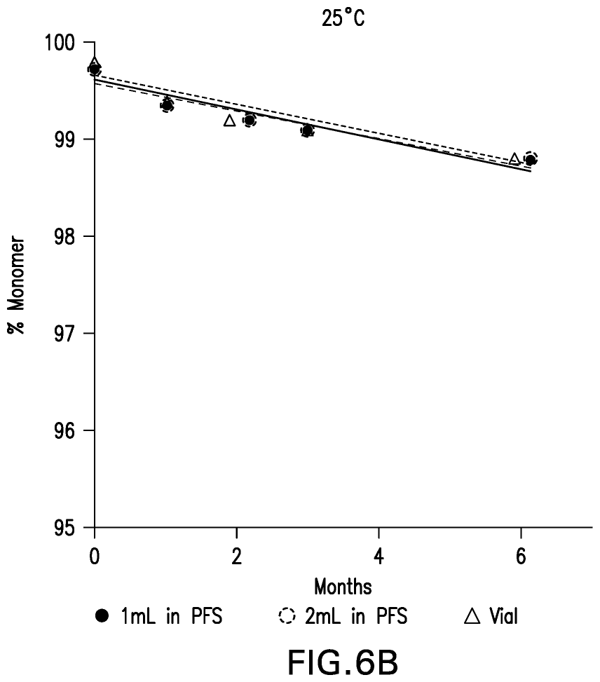

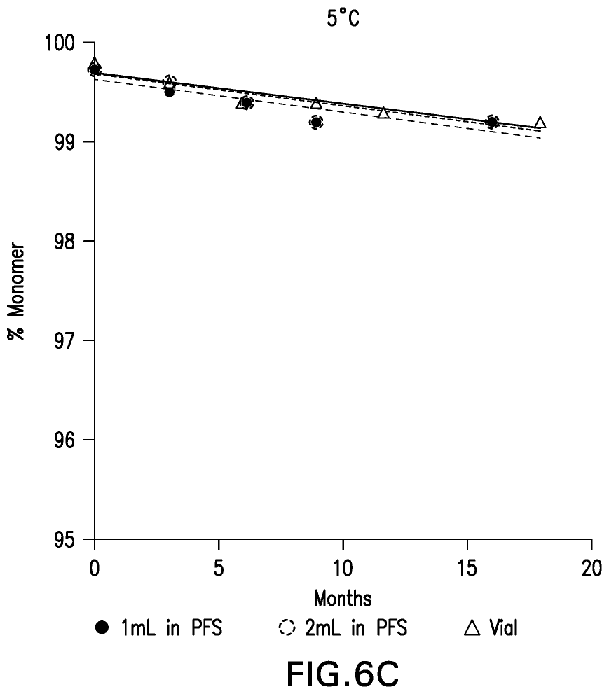

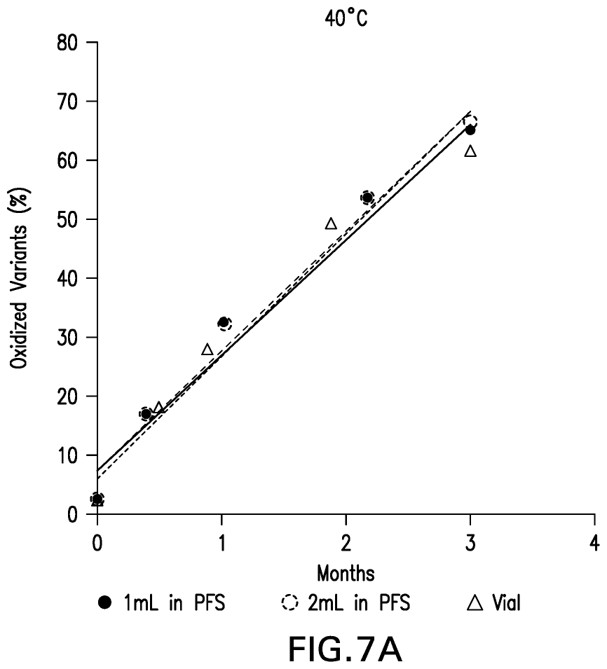

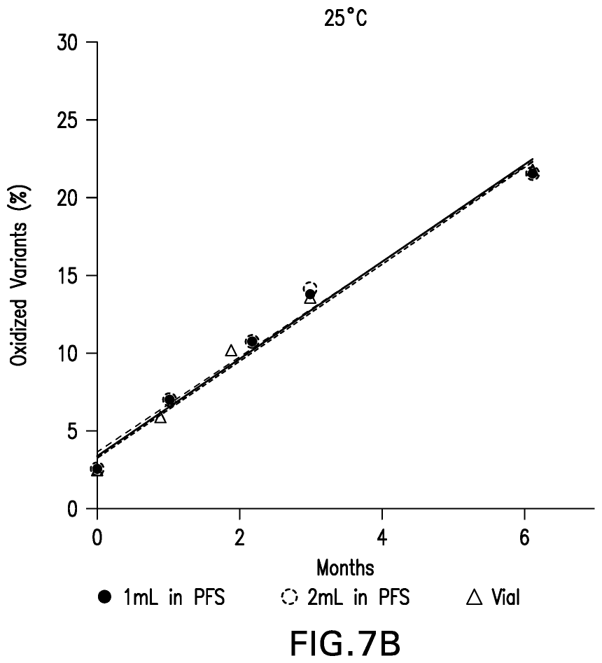

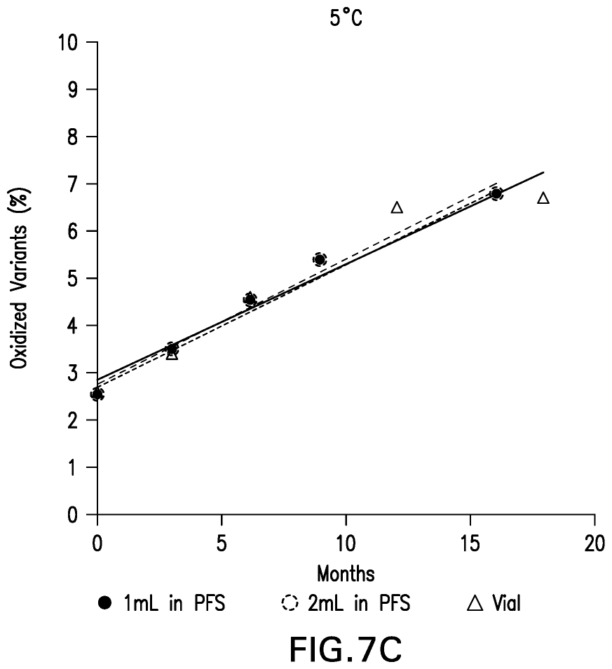

At all temperatures, it was determined that the type of storage container (vial or PFS) for anti-ILT7 mAb formulations did not impact percent formation of oxidation variants (FIGS. 6A-6C) or promote a decrease in monomeric percentage (e.g., increase aggregation; FIGS. 7A-7C). Additionally, the volume of anti-ILT7 mAb formulation (1 mL or 2 mL) in the PFS did not impact either percent oxidation or percent monomers. These data show that a PFS can be used as a storage container for an anti-ILT7 mAb formulation at both 1 mL and 2 mL volume, and obviates the engineering need to use vials as storage containers.

Accordingly, any of the provided storage containers, such as pre-filled autoinjectors, can be utilized without comprising performance of the disclosed ILT7 antibody compositions.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

International patent applications: PCT/US2016/022003, PCT/JP2006/325391, PCT/US2017/021616, PCT/US2020/063396, and PCT/US2022/027620 are all hereby incorporated by reference in their entireties for all intents and purposes.

1. A composition, comprising:
(i) about 100 mg/mL to about 450 mg/mL of an immunoglobulin-like transcript 7 (ILT7) binding protein;
(ii) about 50 to about 350 mM sucrose;
(iii) about 0.001% to about 1% polysorbate-80;
(iv) L-methionine; and
(v) a buffer selected from a histidine buffer, a citrate buffer, or a phosphate buffer.

2. A composition, comprising:
(i) about 100 mg/mL to about 165 mg/mL of an immunoglobulin-like transcript 7 (ILT7) binding protein;
(ii) about 150 to about 240 mM sucrose;
(iii) about 0.003% to about 0.8% polysorbate-80;
(iv) L-methionine; and
(v) a buffer selected from a histidine buffer, a citrate buffer, or a phosphate buffer.

3. The composition of any one of claims 1-2, wherein the buffer is the histidine buffer.

4. The composition of any one of claims 1-2, wherein the buffer is the phosphate buffer.

5. The composition of any one of claims 1-2, wherein the buffer is the citrate buffer.

6. The composition of any one of claims 1-5, wherein a pH of the composition is acidic as determined by a pH meter.

7. The composition of claim 6, wherein the acidic pH is from about 5 to about 7 as determined by pH meter.

8. The composition of claim 7, wherein the acidic pH is about 6 as determined by pH meter.

9. The composition of any one of claims 1-8, wherein the composition comprises from about 5 mM to about 25 mM histidine/histidine-HCl.

10. The composition of claim 9, comprising about 20 mM histidine/histidine-HCl.

11. The composition of any one of claims 1-10, wherein the composition comprises about 5 mM to about 50 mM L-methionine.

12. The composition of claim 11, comprising about 15 mM L-methionine.

13. The composition of any one of claims 1-12, comprising about 0.02% polysorbate-80.

14. The composition of any one of claims 1-13, comprising up to about 150 mg/mL of the ILT7 binding protein.

15. The composition of any one of claims 1-14, comprising about 150 mg/mL of the ILT7 binding protein.

16. The composition of any one of claims 1-15, wherein the ILT7 binding protein comprises: i. a VH comprising at least 95%, 96%, 97%, 98% or 99% identity to SEQ ID NO: 1;
ii. a VL comprising at least 95%, 96%, 97%, 98% or 99% identity to SEQ ID NO: 2; or
iii. i. and ii.

17. The composition of any one of claims 1-16, wherein the ILT7 binding protein comprises: i. a VH comprising SEQ ID NO:1;
ii. a VL comprising SEQ ID NO: 2; or
iii. i. and ii.

18. The composition of any one of claims 1-17, wherein the ILT7 binding protein comprises Complementarity-Determining Regions (CDRs) HCDR1, HDR2, HCDR3, LCDR1, LCDR2, and LCDR3 comprising the sequences of SEQ ID NOs: 3, 4, 5, 6, 7, and 8, respectively.

19. The composition of any one of claim 1-18, wherein the composition comprises a viscosity of about 5 cP to about 50 cP as determined by a viscometer.

20. The composition of any one of claims 1-19, wherein the ILT7 binding protein is daxdilimab.

21. A composition, comprising about:
i. 150 mg/mL of an immunoglobulin-like transcript 7 (ILT7) binding protein;
ii. 20 mM histidine/histidine-HCl;
iii. 180 mM Sucrose;
iv. 15 mM L-Methionine; and
v. 0.02% Polysorbate-80.

22. A composition, comprising about:
i. 150 mg/mL of an immunoglobulin-like transcript 7 (ILT7) binding protein;
ii. 20 mM histidine/histidine-HCl;
iii. 180 mM Sucrose;
iv. 15 mM L-Methionine; and
v. 0.02% Polysorbate-80, wherein the ILT7 binding protein comprises: a VH comprising SEQ ID NO: 1 and a VL comprising SEQ ID NO: 2.

23. A composition, comprising about:
i. 150 mg/mL of an immunoglobulin-like transcript 7 (ILT7) binding protein;
ii. 20 mM histidine/histidine-HCl;
iii. 180 mM Sucrose;
iv. 15 mM L-Methionine; and
v. 0.02% Polysorbate-80, wherein the ILT7 binding protein comprises Complementarity-Determining Regions (CDRs) HCDR1, HDR2, HCDR3, LCDR1, LCDR2, and LCDR3 comprising the sequences of SEQ ID NOs: 3, 4, 5, 8, 7, and 8, respectively.

24. The composition of any one of claims 21-23, wherein viscosity of the composition is from about 5 to about 50 cP.

25. The composition of claim 24, wherein the viscosity does not exceed 20 cP.

26. The composition of any one of claims 21-25, wherein the rate of aggregation is reduced as compared to an otherwise comparable composition lacking the L-Methionine as determined by size exclusion chromatography at 40° C.

27. The composition of any one of claims 21-26, wherein the rate of aggregation is from 0.5% to 1% per month as determined by size exclusion chromatography at 40° C.

28. The composition of any one of claims 21-27, wherein a level of oxidation is reduced as compared to an otherwise comparable composition lacking the L-Methionine as determined by peptide mapping of W102 or W104 of SEQ ID NO: 1 of the ILT7 binding protein at room temperature.

29. The composition of any one of claims 21-28, wherein when the composition is exposed to light at 1600-1800 lux for 5 days, the increase in percentage of oxidized species of ILT7 binding protein in the composition is at most 1.0% as determined by size exclusion chromatography.

30. The composition of any one of claims 21-29, wherein the composition is Fc oxidation-resistant.

31. A container comprising the composition of any one of claims 1-30 in unit dose form.

32. The container of claim 31, wherein the container comprises nitrogen.

33. The container of claim 31, wherein the container does not comprise nitrogen.

34. A method of treating a disease comprising administering an effective amount of the composition of any one of claims 1-30 to a subject in need thereof, thereby treating the disease.

35. The method of claim 34, wherein the administering is sufficient to reduce a symptom of the disease in the subject in need thereof after the administering.

36. The method of claim 35, wherein the symptom is reduced by at least about 1-fold.

37. A method of treating an autoimmune disease comprising administering an effective amount of the composition of any of claims 1-30 to a subject in need thereof, thereby treating the autoimmune disease.

38. The method of claim 37, wherein the autoimmune disease is selected from the group consisting of: Discoid lupus erythematosus (DLE), Focal segmental glomerulosclerosis (FSGS), alopecia areata, myositis, lupus nephritis, diabetes, Hashimoto's disease, autoimmune adrenal insufficiency, pure red cell anemia, multiple sclerosis, rheumatoid carditis, systemic lupus erythematosus, psoriasis, rheumatoid arthritis, chronic inflammation, Sjogren's syndrome, polymyositis, dermatomyositis, inclusion body myositis, juvenile myositis, IgG4 related disease (IgG4RD), and scleroderma.

39. The method of claim 38, wherein the autoimmune disease is alopecia areata.

40. The method of claim 38, wherein the autoimmune disease is systemic lupus erythematosus.

41. The method of claim 38, wherein the autoimmune disease is FSGS.

42. A method of treating alopecia areata comprising administering an effective amount of the composition of any of claims 1-30 to a subject in need thereof, thereby treating the alopecia areata.

43. A method of treating Focal segmental glomerulosclerosis (FSGS) comprising administering an effective amount of the composition of any of claims 1-30 to a subject in need thereof, thereby treating the FSGS.

44. A method of treating systemic lupus erythematosus comprising administering an effective amount of the composition of any of claims 1-30 to a subject in need thereof, thereby treating the systemic lupus erythematosus.

45. The method of claim 44, wherein the administering is every 4 weeks.

46. The method of claim 44, wherein the administering is every 12 weeks.

47. A method of treating myositis comprising administering an effective amount of the composition of any of claims 1-30 to a subject in need thereof, thereby treating the myositis.

48. The method of any one of claims 37-47, wherein the administering is subcutaneous.

49. A composition, comprising about:
i. 150 mg/mL of an immunoglobulin-like transcript 7 (ILT7) binding protein;
ii. 20 mM histidine/histidine-HCl;
iii. 180 mM Sucrose;
iv. 15 mM L-Methionine; and
v. 0.02% Polysorbate-80, wherein the ILT7 binding protein comprises a sequence having at least 85% identity to a sequence of Table 1.

50. A composition, comprising about:
i. 150 mg/mL of an immunoglobulin-like transcript 7 (ILT7) binding protein;
ii. 20 mM histidine/histidine-HCl;
iii. 180 mM Sucrose;
iv. 15 mM L-Methionine; and
v. 0.02% Polysorbate-80, wherein the ILT7 binding protein comprises a VH comprising SEQ ID NO:1, a VL comprising SEQ ID NO: 2, or both the VH and VL of SEQ ID NO: 1 and SEQ ID NO: 2, respectively.

51. A method of treating an autoimmune disease comprising administering a composition that comprises:
i. about 150 mg/mL of an immunoglobulin-like transcript 7 (ILT7) binding protein;
ii. 20 mM histidine/histidine-HCl;
iii. 180 mM Sucrose;
iv. 15 mM L-Methionine; and
v. 0.02% Polysorbate-80,
wherein the ILT7 binding protein comprises Complementarity-Determining Regions (CDRs) HCDR1, HDR2, HCDR3, LCDR1, LCDR2, and LCDR3 comprising the sequences of SEQ ID NOs: 3, 4, 5, 6, 7, and 8 respectively, wherein the dose of the ILT7 binding protein comprises from about 150 mg to about 450 mg; wherein the administration is subcutaneous, and wherein the dose is administered in a single injection.

52. The method of claim 51, wherein the dose of the ILT7 binding protein comprises from about 150 mg to about 275 mg.

53. The method of claim 52, wherein the dose of the ILT7 binding protein comprises from about 150 mg to about 200 mg.

54. A delivery device comprising the composition of any one of claim 1-30, or 49-50.

55. The delivery device of claim 54, wherein the device is an autoinjector.

56. A kit comprising the delivery device of any one of claims 54-55, and instructions for use thereof.

57. The kit of claim 56, comprising a plurality of autoinjectors.

58. A method of treatment, comprising administering daxdilimab to a subject in need thereof, wherein the administering comprises use of an autoinjector.

59. The method of treatment of claim 58, wherein the autoinjector is contacted with the thigh, abdomen, or arm of the subject in a manner sufficient to effectuate the administration of the daxdilimab to the subject in need.

60. The method of any one of claims 58-59, wherein the treatment is of an autoimmune disease or condition selected from the group consisting of: Discoid lupus erythematosus (DLE), Focal segmental glomerulosclerosis (FSGS), alopecia areata, lupus nephritis, myositis, diabetes, Hashimoto's disease, autoimmune adrenal insufficiency, pure red cell anemia, multiple sclerosis, rheumatoid carditis, systemic lupus erythematosus, psoriasis, rheumatoid arthritis, chronic inflammation, Sjogren's syndrome, polymyositis, dermatomyositis, inclusion body myositis, juvenile myositis, IgG4 related disease (IgG4RD), and scleroderma.

61. The method of any one of claims 58-59, wherein the treatment is of a non-autoimmune disease or condition.

62. The method of claim 61, wherein the non-autoimmune disease or condition comprises an ILT7-expressing cell-mediated disease.

63. A composition, comprising:
(i) an immunoglobulin-like transcript 7 (ILT7) binding protein; and
(ii) L-methionine; wherein when the composition is exposed to light at 1600-1800 lux for 5 days, the increase in percentage of oxidized species of ILT7 binding protein in the composition is at most about 1.0% as determined by size exclusion chromatography.

64. The composition of claim 63, wherein the increase in percentage of oxidized species of ILT7 binding protein in the composition is at most about 0.4%, 0.3%, 0.2%, or 0.1%.

65. A composition, comprising:
(i) an immunoglobulin-like transcript 7 (ILT7) binding protein; and
(ii) L-methionine; wherein when the composition is exposed to light at 1600-1800 lux the rate of formation of new oxidized species per day of ILT7 binding protein in the composition is at most about 0.1%-0.5% as determined by size exclusion chromatography.

66. The composition of claim 65, wherein the rate of formation of new oxidized species per day of the ILT7 binding protein in the composition is at most about 0.2%-0.4% or 0.1%-0.3%.

67. The composition of any one of claims 63-66, comprising about 100 mg/mL to about 450 mg/mL of the ILT7 binding protein.

68. The composition of any one of claims 63-67, wherein the ILT7 binding protein comprises Complementarity-Determining Regions (CDRs) HCDR1, HDR2, HCDR3, LCDR1, LCDR2, and LCDR3 comprising the sequences of SEQ ID NOs: 3, 4, 5, 6, 7, and 8, respectively.
